(19) United States
(12) Patent Application Publication
(10) Pub. No.: US 2025/0289872 A1
(43) Pub. Date: Sep. 18, 2025

(54) FORMULATION FOR ANTIGEN-BINDING MOLECULES THAT BIND TO PORPHYROMONAS GINGIVALIS

(71) Applicant: K-Bio Investment, LLC, Austin, TX (US)

(72) Inventors: Peter L. NARA, St. Louis, MO (US); Indraneel SANYAL, St. Louis, MO (US); Tnv GANESHKUMAR, St. Louis, MO (US)

(21) Appl. No.: 18/860,288

(22) PCT Filed: Apr. 24, 2023
(86) PCT No.: PCT/US2023/066125

§ 371 Date: Oct. 25, 2024

(30) Foreign Application Priority Data
Apr. 26, 2022 (IN) 202211024511

(51) Int. Cl. C07K 16/12; A61K 39/395; A61K 47/10; A61K 47/22; A61K 47/26; A61K 47/60

(52) U.S. Cl. CPC C07K 16/1203; A61K 39/39591; A61K 47/10; A61K 47/22; A61K 47/26; A61K 47/60; C07K 2317/24; C07K 2317/94

(57) ABSTRACT

Formulations for antibodies and/or antigen-binding molecules (ABMs) that bind to Porphyromonas gingivalis are described. The formulations may be used in treating infections involving P. gingivalis, such as periodontal disease. The formulations comprise at least one of histidine, saline, acetic acid, citrate phosphate, sucrose, and/or Poloxamer P188. In some embodiments, the formulations may also be pH adjusted.

### CROSS REFERENCE TO RELATED APPLICATIONS

This application claims priority to Indian Prov. App. No. 202211024511, filed Apr. 26, 2022, entitled “FORMULATION FOR ANTIGEN-BINDING MOLECULES THAT BIND TO PORPHYROMONAS GINGIVALIS”, which is incorporated by reference in its entirety.

### REFERENCE TO SEQUENCE LISTING

The present application is being filed along with a Sequence Listing in electronic format. The Sequence Listing is provided as a file entitled “KEYBI.016WO.xml” created on Apr. 17, 2023, which is 6,908 bytes in size. The information in the electronic format of the Sequence Listing is incorporated herein by reference in its entirety.

### BACKGROUND

### Field

The present disclosure generally relates to a formulation for antigen-binding molecules that bind to Porphyromonas gingivalis.

### Description of the Related Art

Porphyromonas gingivalis is a gram-negative anaerobic, asaccharolytic, red complex bacteria. P. gingivalis can infect and remain permanently in the oral cavity as a polymicrobial biofilm and/or translocate to other body cells/tissues. Upon infection, P. gingivalis can produce and excrete outer membrane vesicles (containing gingipains, hemagglutinin, adhesins and LPS) into the gingival sulcus space with its attending fluid, blood and lymphatic circulation. As disclosed herein, the regularly distributed polyclonal bio-film colonies of P. gingivalis are deeper in the sulcular tissues and extracellular portions of the oral cavity, while the OMVs produced by P. gingivalis are more diffusely spread to surrounding tissues and in the GCF/lymph and micro-vascular systems. P. gingivalis infection can lead to a state of oral and systemic dysbiosis (pathological and abnormal change from the normal oral flora/microbiota) and subsequent chronic local and systemic infection/disease(s), further leading to increased vascular and tissue inflammation throughout the entire body. Certain end organs, e.g., heart vessels, carotid arteries, vessels in the brain, liver, joints, lungs, pancreas, reproductive system, etc., are more affected than others. P. gingivalis-induced inflammation is implicated in diseases such as cardiovascular disease, heart attacks, atherosclerosis, stroke, various dementias, early and later neuro-cognitive decline, Alzheimer's disease, diabetes, NASH, rheumatoid arthritis, insulin resistance, etc.

### SUMMARY

Disclosed herein is an antibody formulation comprising histidine and an antibody, wherein the antibody targets P. gingivalis. In some embodiments, the antibody comprises at least one, two, three, four, five, or all six of the CDR regions of SEQ ID NOS: 1-2. In some embodiments, the antibody comprises at least one CDR region that has at least 75%, 79%, or 80% identity to one, two, three, four, five, or all six of the CDR regions of SEQ ID NOS: 1-2, respectively. In some embodiments, the antibody comprises a VH of a VH within SEQ ID NO: 1 and a VL of a VL within of SEQ ID NO: 2. In some embodiments, the antibody comprises a VH that has at least 75%, 79%, or 80% identity to a VH within SEQ ID NO: 1, and a VL that has at least 75%, 79%, or 80% identity to a VL within of SEQ ID NO: 2. In some embodiments, the antibody is present at a concentration of about 0.9-0.12 mg/mL. In some embodiments, the antibody is present at a concentration of 0.1 mg/mL. In some embodiments, the histidine is present at a concentration of about 1 to about 40 mM. In some embodiments, the histidine is present at a concentration of 20 mM. In some embodiments, the formulation has a pH of about 4 to about 8. In some embodiments, the formulation has a pH of 6. In some embodiments, the formulation has a pH of 5.8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises acetic acid (acetate) at a concentration of about 1 to about 20 mM. In some embodiments, the formulation further comprises acetic acid (acetate) at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, the formulation further comprises sucrose at a concentration of about 85 to 200 mM. In some embodiments, the formulation further comprises sucrose at a concentration of about 85 mM. In some embodiments, the formulation further comprises sucrose at a concentration of about 150 mM. In some embodiments, the formulation further comprises sucrose at a concentration of about 200 mM.

Also disclosed herein is an antibody formulation comprising histidine and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO. 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO. 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises a VH that has at least 75%, 79%, or 80% identity to a VH within SEQ ID NO: 1, and a VL that has at least 75%, 79%, or 80% identity to a VL within of SEQ ID NO: 2. In some embodiments, the antibody comprises SEQ ID NO: 1 and SEQ ID NO: 2. In some embodiments, the antibody comprises a sequence that has at least 75%, 79%, or 80% identity to SEQ ID NO: 1. In some embodiments, the antibody comprises a sequence that has at least 75%, 79%, or 80% identity to SEQ ID NO: 2. In some embodiments, the antibody comprises a sequence that has at least 75%, 79%, or 80% identity to SEQ ID NO: 1, and further comprises a sequence that has at least 75%, 79%, or 80% identity to SEQ ID NO: 2. In some embodiments, the antibody is present at a concentration of about 0.9-0.12 mg/mL. In some embodiments, the antibody is present at a concentration of 0.1 mg/mL. In some embodiments, the histidine is present at a concentration of about 1 to about 40 mM. In some embodiments, the histidine is present at a concentration of 20 mM. In some embodiments, the formulation has a pH of about 4 to about 8. In some embodiments, the formulation has a pH of 6. In some embodiments, the formulation has a pH of 5.8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises acetic acid (acetate) at a concentration of about 1 to about 20 mM. In some embodiments, the formulation further comprises acetic acid (acetate) at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, the formulation further comprises sucrose at a concentration of about 85 to 200 mM. In some embodiments, the formulation further comprises sucrose at a concentration of about 85 mM. In some embodiments, the formulation further comprises sucrose at a concentration of about 150 mM. In some embodiments, the formulation further comprises sucrose at a concentration of about 200 mM.

Also disclosed herein is an antibody formulation comprising citrate phosphate and an antibody, wherein the antibody targets P. gingivalis. In some embodiments, the antibody comprises at least one, two, three, four, five, or all six of the CDR regions of SEQ ID NOS: 1-2. In some embodiments, the antibody comprises at least one CDR region that has at least 75%, 79%, or 80% identity to one, two, three, four, five, or all six of the CDR regions of SEQ ID NOS: 1-2, respectively. In some embodiments, the antibody comprises a VH of a VH within SEQ ID NO: 1 and a VL of a VL within of SEQ ID NO: 2. In some embodiments, the antibody comprises a VH that has at least 75%, 79%, or 80% identity to a VH within SEQ ID NO: 1, and a VL that has at least 75%, 79%, or 80% identity to a VL within of SEQ ID NO: 2. In some embodiments, the antibody is present at a concentration of about 0.1 mg/mL. In some embodiments, the citrate phosphate is present at a concentration of about 1 to about 20 mM. In some embodiments, the citrate phosphate is present at a concentration of about 10 mM. In some embodiments, the formulation has a pH of about 4 to about 8. In some embodiments, the formulation has a pH of 6. In some embodiments, the formulation has a pH of 5.8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises acetic acid (acetate) at a concentration of about 1 to about 20 mM. In some embodiments, the formulation further comprises acetic acid (acetate) at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, In some embodiments, the formulation further comprises sucrose at a concentration of about 85 to 200 mM. In some embodiments, the formulation further comprises sucrose at a concentration of about 85 mM. In some embodiments, In some embodiments, the formulation further comprises sucrose at a concentration of about 150 mM. In some embodiments, the formulation further comprises sucrose at a concentration of about 200 mM.

Also disclosed herein is an antibody formulation comprising citrate phosphate and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises a VH that has at least 75%, 79%, or 80% identity to a VH within SEQ ID NO: 1, and a VL that has at least 75%, 79%, or 80% identity to a VL within of SEQ ID NO: 2. In some embodiments, the antibody comprises SEQ ID NO: 1 and SEQ ID NO: 2. In some embodiments, the antibody comprises a sequence that has at least 75%, 79%, or 80% identity to SEQ ID NO: 1. In some embodiments, the antibody comprises a sequence that has at least 75%, 79%, or 80% identity to SEQ ID NO: 2. In some embodiments, the antibody comprises a sequence that has at least 75%, 79%, or 80% identity to SEQ ID NO: 1, and further comprises a sequence that has at least 75%, 79%, or 80% identity to SEQ ID NO: 2. In some embodiments, the antibody is present at a concentration of about 0.1 mg/mL. In some embodiments, the citrate phosphate is present at a concentration of about 1 to about 20 mM. In some embodiments, the citrate phosphate is present at a concentration of about 10 mM. In some embodiments, the formulation has a pH of about 4 to about 8. In some embodiments, the formulation has a pH of 6. In some embodiments, the formulation has a pH of 5.8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises acetic acid (acetate) at a concentration of about 1 to about 20 mM. In some embodiments, the formulation further comprises acetic acid (acetate) at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, In some embodiments, the formulation further comprises sucrose at a concentration of about 85 to 200 mM. In some embodiments, the formulation further comprises sucrose at a concentration of about 85 mM. In some embodiments, In some embodiments, the formulation further comprises sucrose at a concentration of about 150 mM. In some embodiments, the formulation further comprises sucrose at a concentration of about 200 mM.

An antibody formulation comprising acetic acid (acetate) and an antibody, wherein the antibody targets P. gingivalis. In some embodiments, the antibody comprises at least one, two, three, four, five, or all six of the CDR regions of SEQ ID NOS: 1-2. In some embodiments, the antibody comprises a VH of a VH within SEQ ID NO: 1 and a VL of a VL within of SEQ ID NO: 2. In some embodiments, the antibody is present at a concentration of about 0.1 mg/mL. In some embodiments, the acetic acid (acetate) is present at a concentration of about 1 to about 20 mM. In some embodiments, the acetic acid (acetate) is present at a concentration of about 10 mM. In some embodiments, the formulation has a pH of about 4 to about 8. In some embodiments, the formulation has a pH of 5. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, the formulation further comprises sucrose at a concentration of about 85 to 200 mM. In some embodiments, the formulation further comprises sucrose at a concentration of about 85 mM. In some embodiments, the formulation further comprises sucrose at a concentration of about 150 mM. In some embodiments, the formulation further comprises sucrose at a concentration of about 200 mM.

Also disclosed herein is an antibody formulation comprising acetic acid (acetate) and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO:1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises SEQ ID NO: 1 and SEQ ID NO: 2. In some embodiments, the antibody is present at a concentration of about 0.1 mg/mL. In some embodiments, the acetic acid (acetate) is present at a concentration of about 1 to about 20 mM. In some embodiments, the acetic acid (acetate) is present at a concentration of about 10 mM. In some embodiments, the formulation has a pH of about 4 to about 8. In some embodiments, the formulation has a pH of 5. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, the formulation further comprises sucrose at a concentration of about 85 to 200 mM. In some embodiments, the formulation further comprises sucrose at a concentration of about 85 mM. In some embodiments, the formulation further comprises sucrose at a concentration of about 150 mM. In some embodiments, the formulation further comprises sucrose at a concentration of about 200 mM.

Also disclosed herein is an antibody formulation comprising a Poloxamer P188 and an antibody, wherein the antibody targets P. gingivalis. In some embodiments, the antibody comprises at least one, two, three, four, five, or all six of the CDR regions of SEQ ID NOS: 1-2. In some embodiments, the antibody comprises a VH of a VH within SEQ ID NO: 1 and a VL of a VL within of SEQ ID NO: 2. In some embodiments, the antibody is present at a concentration of about 0.1 mg/mL. In some embodiments, the Poloxamer P188 is present at a concentration of about 0.001-0.1% weight/volume. In some embodiments, the Poloxamer P188 is present at a concentration of about 0.01% weight/volume. In some embodiments, the formulation has a pH of about 4 to about 8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises acetic acid (acetate) at a concentration of about 1 to about 20 mM. In some embodiments, the formulation further comprises acetic acid (acetate) at a concentration of about 10 mM. the formulation further comprises sucrose at a concentration of about 85 to 200 mM. In some embodiments, the formulation further comprises sucrose at a concentration of about 85 mM. In some embodiments, the formulation further comprises sucrose at a concentration of about 150 mM. In some embodiments, the formulation further comprises sucrose at a concentration of about 200 mM.

Also disclosed herein is an antibody formulation comprising Poloxamer P188 and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises SEQ ID NO: 1 and SEQ ID NO: 2. In some embodiments, the antibody is present at a concentration of about 0.1 mg/mL. In some embodiments, the Poloxamer P188 is present at a concentration of about 0.001-0.1% weight/volume. In some embodiments, the Poloxamer P188 is present at a concentration of about 0.01% weight/volume. In some embodiments, the formulation has a pH of about 4 to about 8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises acetic acid (acetate) at a concentration of about 1 to about 20 mM. In some embodiments, the formulation further comprises acetic acid (acetate) at a concentration of about 10 mM. the formulation further comprises sucrose at a concentration of about 85 to 200 mM. In some embodiments, the formulation further comprises sucrose at a concentration of about 85 mM. In some embodiments, the formulation further comprises sucrose at a concentration of about 150 mM. In some embodiments, the formulation further comprises sucrose at a concentration of about 200 mM.

Also disclosed herein is an antibody formulation comprising a sucrose and an antibody, wherein the antibody targets P. gingivalis. In some embodiments, the antibody comprises at least one, two, three, four, five, or all six of the CDR regions of SEQ ID NOS: 1-2. In some embodiments, the antibody comprises a VH of a VH within SEQ ID NO: 1 and a VL of a VL within of SEQ ID NO: 2. In some embodiments, the antibody is present at a concentration of about 0.1 mg/mL In some embodiments, the sucrose is present at a concentration of about 85 to 200 mM. In some embodiments, the sucrose is present at a concentration of about 85 mM. In some embodiments, the sucrose is present at a concentration of about 150 mM. In some embodiments, the sucrose is present at a concentration of about 200 mM. In some embodiments, the formulation has a pH of about 4 to about 8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises acetic acid (acetate) at a concentration of about 1 to about 20 mM. In some embodiments, the formulation further comprises acetic acid (acetate) at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume.

Also disclosed herein is an antibody formulation comprising a sucrose and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises SEQ ID NO: 1 and SEQ ID NO: 2. In some embodiments, the antibody is present at a concentration of about 0.1 mg/mL. In some embodiments, the sucrose is present at a concentration of about 85 to 200 mM. In some embodiments, the sucrose is present at a concentration of about 85 mM. In some embodiments, the sucrose is present at a concentration of about 150 mM. In some embodiments, the sucrose is present at a concentration of about 200 mM. In some embodiments, the formulation has a pH of about 4 to about 8. In some embodiments, the formulation further comprises saline (sodium chloride) at a concentration of about 0.1-2.0% weight/volume. In some embodiments, the formulation further comprises saline (sodium chloride) at a concentration of about 0.9% weight/volume. In some embodiments, the formulation further comprises histidine at a concentration of about 1 to about 40 mM. In some embodiments, the formulation further comprises histidine at a concentration of 20 mM. In some embodiments, the formulation further comprises citrate phosphate at a concentration of about 1 to about 20 mM. In some embodiments, the formulation further comprises citrate phosphate at a concentration of about 10 mM. In some embodiments, the formulation further comprises acetic acid (acetate) at a concentration of about 1 to about 20 mM. In some embodiments, the formulation further comprises acetic acid (acetate) at a concentration of about 10 mM. In some embodiments, the formulation further comprises Poloxamer P188 at a concentration of about 0.001-0.1% weight/volume. In some embodiments, the formulation further comprises Poloxamer P188 at a concentration of about 0.01% weight/volume.

Also disclosed herein is an antibody formulation comprising saline, sucrose, Poloxamer P188, and an antibody, wherein the antibody targets P. gingivalis. In some embodiments, the antibody comprises at least one, two, three, four, five, or all six of the CDR regions of SEQ ID NOS: 1-2. In some embodiments, the antibody comprises a VH of a VH within SEQ ID NO: 1 and a VL of a VL within of SEQ ID NO. 2. In some embodiments, the antibody is present at a concentration of about 0.1 mg/mL. In some embodiments, saline is present at a concentration of about 0.1-2.0% weight/volume. In some embodiments, saline is present at a concentration of about 0.9% weight/volume. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, the sucrose is present at a concentration of about 85 to 200 mM. In some embodiments, the sucrose is present at a concentration of about 85 mM. In some embodiments, the sucrose is present at a concentration of about 150 mM. In some embodiments, the sucrose is present at a concentration of about 200 mM.

Also disclosed herein is an antibody formulation comprising saline, sucrose, Poloxamer P188, and an antibody, wherein the antibody comprises at least one, two, three, four, five, six, seven, or all eight of, a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises SEQ ID NO: 1 and SEQ ID NO: 2. In some embodiments, the antibody is present at a concentration of about 0.1 mg/mL. In some embodiments, saline is present at a concentration of about 0.1-2.0% weight/volume. In some embodiments, saline is present at a concentration of about 0.9% weight/volume. In some embodiments, the formulation further comprises Poloxamer P188 at a concentration of about 0.001-0.1% weight/volume. In some embodiments, the formulation further comprises Poloxamer P188 at a concentration of about 0.01% weight/volume. In some embodiments, the sucrose is present at a concentration of about 85 to 200 mM. In some embodiments, the sucrose is present at a concentration of about 85 mM. In some embodiments, the sucrose is present at a concentration of about 150 mM. In some embodiments, the sucrose is present at a concentration of about 200 mM.

Also disclosed herein is an antibody formulation comprising histidine, sucrose, Poloxamer P188, and an antibody, wherein the antibody targets P. gingivalis. In some embodiments, the antibody comprises at least one, two, three, four, five, or all six of the CDR regions of SEQ ID NOS: 1-2. In some embodiments, the antibody comprises a VH of a VH within SEQ ID NO: 1 and a VL of a VL within of SEQ ID NO: 2. In some embodiments, histidine is present at a concentration of about 1 to about 40 mM. In some embodiments, histidine is present at a concentration of about 20 mM. In some embodiments, the formulation has a pH of about 4 to about 8. In some embodiments, the formulation has a pH of 6. In some embodiments, the formulation has a pH of 5.8. In some embodiments, the Poloxamer P188 is present at a concentration of about 0.001-0.1% weight/volume. In some embodiments, the Poloxamer P188 is present at a concentration of about 0.01% weight/volume. In some embodiments, the sucrose is present at a concentration of about 85 to 200 mM. In some embodiments, the sucrose is present at a concentration of about 85 mM. In some embodiments, the sucrose is present at a concentration of about 150 mM. In some embodiments, the sucrose is present at a concentration of about 200 mM.

Also disclosed herein is an antibody formulation comprising histidine, sucrose, Poloxamer P188, and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some embodiments, the antibody targets P. gingivalis. In some embodiments, the antibody comprises a VH of a VH within SEQ ID NO: 1 and a VL of a VL within of SEQ ID NO: 2. In some embodiments, the antibody comprises SEQ ID NO: 1 and SEQ ID NO. 2. In some embodiments, histidine is present at a concentration of about 1 to about 40 mM. In some embodiments, histidine is present at a concentration of about 20 mM. In some embodiments, the formulation has a pH of about 4 to about 8. In some embodiments, the formulation has a pH of 6. In some embodiments, the formulation has a pH of 5.8. In some embodiments, the Poloxamer P188 is present at a concentration of about 0.001-0.1% weight/volume. In some embodiments, the Poloxamer P188 is present at a concentration of about 0.01% weight/volume. In some embodiments, the sucrose is present at a concentration of about 85 to 200 mM. In some embodiments, the sucrose is present at a concentration of about 85 mM. In some embodiments, the sucrose is present at a concentration of about 150 mM. In some embodiments, the sucrose is present at a concentration of about 200 mM.

In some embodiments, the antibody in the formulation is KB001.

Also disclosed herein is a method of storing an antibody, wherein the method comprises mixing the antibody with an antibody formulations comprising any one of the formulations of the present disclosure. In some embodiments, the antibody formulation is stored at room temperature (about 20-29° C.); most preferably stored at about 25° C. In some embodiments, the antibody formulation is stored at below room temperature; preferably stored between about 20 to about −80° C.; most preferably stored at about 5° C. In some embodiments, the antibody formulation is stored at above room temperature; preferably stored between about 30 to about 80° C.; most preferably stored at about 40° C. In some embodiments, the antibody formulation is stored as a solid and/or frozen. In some embodiments, the antibody formulation is stored as a liquid.

Also disclosed herein is a use of the any one of the antibody formulations of the present disclosure as a storage formulation to enhance the stability of an at least one antibody or antigen binding molecule. In some embodiments, the antibody formulation is stored at room temperature (about 20-29° C.); most preferably stored at about 25° C. In some embodiments, the antibody formulation is stored at below room temperature; preferably stored between about 20 to about −80° C.; most preferably stored at about 5° C. In some embodiments, the antibody formulation is stored at above room temperature; preferably stored between about 30 to about 80° C.; most preferably stored at about 40° C. In some embodiments, the antibody formulation is stored as a solid and/or frozen. In some embodiments, the antibody formulation is stored as a liquid.

### BRIEF DESCRIPTION OF THE DRAWINGS

FIGS. 1A and 1B show the heavy and light chain amino acid sequences, respectively, of KB001 (which includes VH SEQ ID NO: 1 and VL SEQ ID NO: 2). The construct is a mouse construct, which can be used in any of the method embodiments provided herein.

FIG. 2 shows a non-limiting example composition of a formulation for use in storing antibodies. The antibody can be that in FIGS. 1A and 1B (e.g., SEQ ID NOs 1 and 2), with or without the optional material noted in FIG. 2.

### DETAILED DESCRIPTION

Provided herein are formulations for antibodies and/or antigen binding molecules (ABMs), e.g., murine, human-chimeric, human or humanized ABMs, that bind to Porphyromonas gingivalis. The ABMs of the present disclosure can specifically bind to an epitope associated with P. gingivalis, including this certain cell-surface epitope.

The formulation disclosed herein, when including any one of the ABMs, antibodies, or proteins described in the present application, may enhance one or more of the overall stability, delivery, pharmaceutical effectiveness, safety, and/or shelf life of the given ABM, antibody, and/or protein. In some alternatives, the ABM is KB001. In some alternatives, the ABM has a VH and/or VL sequence that is at least 75%, 79%, or 80% identical to SEQ ID NO: 1 and/or 2, respectively. In some alternatives, the ABM has a CDR1 sequence that is at least 75%, 79%, or 80% identical to the CDR1 sequence of SEQ ID NO: 1. In some alternatives, the ABM has a CDR2 sequence that is at least 75%, 79%, or 80% identical to the CDR2 sequence of SEQ ID NO: 1. In some alternatives, the ABM has a CDR3 sequence that is at least 75%, 79%, or 80% identical to the CDR3 sequence of SEQ ID NO: 1. In some alternatives, the ABM has a CDR4 sequence that is at least 75%, 79%, or 80% identical to the CDR4 sequence of SEQ ID NO: 2. In some alternatives, the ABM has a CDR5 sequence that is at least 75%, 79%, or 80% identical to the CDR5 sequence of SEQ ID NO: 2. In some alternatives, the ABM has a CDR6 sequence that is at least 75%, 79%, or 80% identical to the CDR6 sequence of SEQ ID NO: 2. In some alternatives, the ABM has a CDR1, CDR2, and CDR3 sequence that are each at least 75%, 79%, or 80% identical to the CDR1, CDR2, and CDR3 sequences of SEQ ID NO: 1. In some alternatives, the ABM has a CDR4, CDR5, and CDR6 sequence that are each at least 75%, 79%, or 80% identical to the CDR4, CDR5,a and CDR6 sequences of SEQ ID NO: 2. In some embodiments, the formulation is formulated to have a final pH that is about 4.0, about 4.5, about 5.0, about 5.5, about 6.0, about 6.5, about 7.0, about 7.5 or any pH value that is in between about 4.0 and about 7.5. As will be appreciated by one skilled in the art, the pH of the formulation may be adjusted using the titration of any acid or base, alone or in combination with any other acid or base. In some alternatives, the acid is HCl and/or phosphate. In some alternatives, the base is KOH and/or NaOH.

In some embodiments, the formulation comprises one or more compounds from the group consisting of: sodium chloride, histidine, histidine HCl, L-histidine, L-histidine HCl, citrate, phosphate, acetic acid/acetate, sucrose, and Poloxamer P188. As will be appreciated by one skilled in the art, these compounds may be alternatively substituted with a close analog or derivative. Non-limiting examples of close analogs and derivatives include D-histidine, D-histidine HCl, oxaloacetate, isocitrate, pyruvate, pyrophosphate, polyphosphate, nitrate, nitrite, glucose, fructose, maltose, lactose, galactose, polysaccharides, PEG, and HEG.

In some embodiments, the formulation comprises sodium chloride and/or saline at about 0.5%, about 0.7%, about 0.9%, about 1%, about 1.5%, about 2% weight/volume, or any integer that is between about 0.5% and about 2% weight/volume.

In some embodiments, the formulation comprises Histidine and/or Histidine HCl at about 5 mM, about 10 mM, about 20 mM, about 30 mM, about 40 mM, about 50 mM, about 100 mM, or any integer that is between about 5 mM and 100 mM.

In some embodiments, the formulation comprises citrate and/or phosphate at about 1 mM, about 5 mM, about 10 mM, about 20 mM, about 50 mM, or any integer that is between about 1 mM and about 50 mM.

In some embodiments, the bu formulation ffer comprises acetic acid/acetate at about 1 mM, about 5 mM, about 10 mM, about 20 mM, about 50 mM, or any integer that is between about 1 mM and about 50 mM.

In some embodiments, the formulation comprises sucrose at about 10 mM, about 25 mM, about 50 mM, about 75 mM, about 85 mM, about 100 mM, about 150 mM, about 200 mM, about 250 mM, about 300 mM, about 500 mM or any concentration that is between about 10 mM and 500 mM. In some alternatives, the formulation comprises Poloxamer P188 at about 0.001%, about 0.005%, about 0.01%, about 0.05%, about 0.1% weight/volume, or any integer that is between about 0.001% and 0.1% weight/volume.

Also disclosed herein is an antibody formulation comprising histidine and an antibody, wherein the antibody targets P. gingivalis. In some alternatives, the antibody comprises at least one, two, three, four, five, or all six of the CDR regions of SEQ ID NOS: 1-2. In some alternatives, the antibody comprises CDR1 of SEQ ID NO:1. In some alternatives, the antibody comprises CDR2 of SEQ ID NO:1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the histidine is present at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises citrate phosphate at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises acetic acid (acetate) at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the formulation further comprises Poloxamer P188 at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising histidine and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO. 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO: 1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO: 2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO: 2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO: 2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the histidine is present at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises citrate phosphate at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises acetic acid (acetate) at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the formulation further comprises Poloxamer P188 at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising citrate phosphate and an antibody, wherein the antibody targets P. gingivalis. In some alternatives, the antibody comprises at least one, two, three, four, five, or all six of the CDR regions of SEQ ID NOS: 1-2. In some alternatives, the antibody comprises CDR1 of SEQ ID NO:1. In some alternatives, the antibody comprises CDR2 of SEQ ID NO: 1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, citrate phosphate is present at a concentration of about 0.1 mM, about 0.5 mM, about 1 mM, about 5 mM, about 10 mM, about 15 mM, about 20 mM, about 25 mM, about 50 mM, or any concentration in between. In some alternatives, citrate phosphate is present at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises acetic acid (acetate) at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the formulation further comprises Poloxamer P188 at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising citrate phosphate and an antibody, wherein the antibody comprises at least one, two, three, four, five, six, seven, or all eight of: a VH segment that is at least 75%, 79%, or 80% identical to SEQ ID NO: 1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO:1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO:2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, citrate phosphate is present at a concentration of about 0.1 mM, about 0.5 mM, about 1 mM, about 5 mM, about 10 mM, about 15 mM, about 20 mM, about 25 mM, about 50 mM, or any concentration in between. In some alternatives, citrate phosphate is present at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises acetic acid (acetate) at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the formulation further comprises Poloxamer P188 at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising acetic acid (acetate) and an antibody, wherein the antibody targets P. gingivalis. In some alternatives, the antibody comprises at least one, two, three, four, five, or all six of the CDR regions of SEQ ID NOS: 1-2. In some alternatives, the antibody comprises CDR1 of SEQ ID NO:1. In some alternatives, the antibody comprises CDR2 of SEQ ID NO:1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises citrate phosphate at a concentration of about 10 mM. In some alternatives, the acetic acid (acetate) is present at a concentration of about 0.1 mM, about 0.5 mM, about 1 mM, about 5 mM, about 10 mM, about 15 mM, about 20 mM, about 25 mM, about 50 mM, or any concentration in between. In some alternatives, the acetic acid (acetate) is present at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the formulation further comprises Poloxamer P188 at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising acetic acid (acetate) and an antibody, wherein the antibody comprises at least one, two, three, four, five, six, seven, or all eight of: a VH segment that is at least 75%, 79%, or 80% identical to SEQ ID NO: 1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO:1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO:2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, citrate phosphate is present in the formulation at a concentration of about 10 mM. In some alternatives, the acetic acid (acetate) is present in the formulation at a concentration of about 0.1 mM, about 0.5 mM, about 1 mM, about 5 mM, about 10 mM, about 15 mM, about 20 mM, about 25 mM, about 50 mM, or any concentration in between. In some alternatives, the acetic acid (acetate) is present in the formulation at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the formulation further comprises Poloxamer P188 at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising Poloxamer P188 and an antibody, wherein the antibody targets P. gingivalis. In some alternatives, the antibody comprises at least one, two, three, four, five, or all six of the CDR regions of SEQ ID NOS: 1-2. In some alternatives, the antibody comprises CDR1 of SEQ ID NO:1 In some alternatives, the antibody comprises CDR2 of SEQ ID NO:1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises citrate phosphate at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the acetic acid (acetate) is present at a concentration of about 10 mM. In some alternatives, the Poloxamer P188 is present in the formulation at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising Poloxamer P188 and an antibody, wherein the antibody comprises at least one, two, three, four, five, six, seven, or all eight of: a VH segment that is at least 75%, 79%, or 80% identical to SEQ ID NO: 1, a VL segment that is at least 75%, 79%, or 80% identical to SEQ ID NO: 2, a CDR1 that is at least 75%, 79%, or 80% identical to the CDR1 of SEQ ID NO: 1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO:1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO:2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 60, about 70, about 8.0, about 8.5, about 90, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, citrate phosphate is present in the formulation at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the acetic acid (acetate) is present in the formulation at a concentration of about 10 mM. In some alternatives, the Poloxamer P188 is present in the formulation at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is present in the formulation at a concentration of about 0.01% weight/volume. In some alternatives, the formulation further comprises sucrose at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the formulation further comprises sucrose at a concentration of about 85 mM. In some alternatives, the formulation further comprises sucrose at a concentration of about 150 mM. In some alternatives, the formulation further comprises sucrose at a concentration of about 200 mM.

Also disclosed herein is an antibody formulation comprising sucrose and an antibody, wherein the antibody targets P. gingivalis. In some alternatives, the antibody comprises at least one, two, three, four, five, or all six of the CDR regions of SEQ ID NOS: 1-2. In some alternatives, the antibody comprises CDR1 of SEQ ID NO:1. In some alternatives, the antibody comprises CDR2 of SEQ ID NO:1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, the formulation further comprises citrate phosphate at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the acetic acid (acetate) is present at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is at a concentration of about 0.01% weight/volume. In some alternatives, the sucrose is present in the formulation at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the sucrose is present in the formulation at a concentration of about 85 mM. In some alternatives, the sucrose is present in the formulation at a concentration of about 150 mM. In some alternatives, the sucrose is present in the formulation at a concentration of about 200 mM.

Also disclosed herein is an antibody formulation comprising sucrose and an antibody, wherein the antibody comprises at least one, two, three, four, five, six, seven, or all eight of: a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO: 1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO: 1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO: 2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO: 2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the formulation further comprises histidine at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the formulation further comprises saline (sodium chloride) at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, or any concentration in between. In some alternatives, the formulation further comprises saline (sodium chloride) at a concentration of about 0.9% weight/volume. In some alternatives, the formulation further comprises citrate phosphate at a concentration of about 0.1 mM, about 0.5 mM, about 1 mM, about 5 mM, about 10 mM, about 15 mM, about 20 mM, about 25 mM, about 50 mM, or any concentration in between. In some alternatives, citrate phosphate is present in the formulation at a concentration of about 10 mM. In some alternatives, the formulation further comprises acetic acid (acetate) at a concentration of about 0.1 mM, about 0.5 mM, about 1 mM, about 5 mM, about 10 mM, about 15 mM, about 20 mM, about 25 mM, about 50 mM, or any concentration in between. In some alternatives, the acetic acid (acetate) is present in the formulation at a concentration of about 10 mM. In some alternatives, the formulation further comprises Poloxamer P188 at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is present in the formulation at a concentration of about 0.01% weight/volume. In some alternatives, the sucrose is present in the formulation at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the sucrose is present in the formulation at a concentration of about 85 mM. In some alternatives, the sucrose is present in the formulation at a concentration of about 150 mM. In some alternatives, the sucrose is present in the formulation at a concentration of about 200 mM.

Also disclosed herein is an antibody formulation comprising saline, sucrose, Poloxamer P188, and an antibody, wherein the antibody targets P. gingivalis. In some alternatives, the antibody comprises at least one, two, three, four, five, or all six of the CDR regions of SEQ ID NOS: 1-2. In some alternatives, the antibody comprises a VH of a VH within SEQ ID NO: 1 and a VL of a VL within of SEQ ID NO: 2. In some alternatives, the antibody comprises at least one, two, three, four, five, or all six of the CDR regions of SEQ ID NOS: 1-2. In some alternatives, the antibody comprises CDR1 of SEQ ID NO:1. In some alternatives, the antibody comprises CDR2 of SEQ ID NO:1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, saline is present in the formulation at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, about 3.0% weight/volume, about 4.0% weight/volume, about 5.0% weight/volume, or any concentration in between. In some alternatives, the saline is present at a concentration of about 0.9% weight/volume. In some alternatives, the sucrose is present in the formulation at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the sucrose is present in the formulation at a concentration of about 85 mM. In some alternatives, the sucrose is present in the formulation at a concentration of about 150 mM. In some alternatives, the sucrose is present in the formulation at a concentration of about 200 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the Poloxamer P188 is present in the formulation at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is at a concentration of about 0.01% weight/volume.

Also disclosed herein is an antibody formulation comprising saline, sucrose, Poloxamer P188, and an antibody, wherein the antibody comprises at least one, two, three, four, five, six, seven, or all eight of a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO: 1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO:1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO:2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/ml, about 10 mg/mL, about 15 mg/mL, about 20 mg/mL, or any concentration that in between. In some alternatives, the antibody is present in the formulation at a concentration of about 0.1 mg/mL. In some alternatives, saline is present in the formulation at a concentration of about 0.01% weight/volume, about 0.05% weight/volume, about 0.1% weight/volume, about 0.5% weight/volume, about 1.0% weight/volume, about 1.5% weight/volume, about 2.0% weight/volume, about 3.0% weight/volume, about 4.0% weight/volume, about 5.0% weight/volume, or any concentration in between. In some alternatives, the saline is present at a concentration of about 0.9% weight/volume. In some alternatives, the sucrose is present in the formulation at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the sucrose is present in the formulation at a concentration of about 85 mM. In some alternatives, the sucrose is present in the formulation at a concentration of about 150 mM. In some alternatives, the sucrose is present in the formulation at a concentration of about 200 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the Poloxamer P188 is present in the formulation at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is at a concentration of about 0.01% weight/volume.

Also disclosed herein is an antibody formulation comprising histidine, sucrose, Poloxamer P188, and an antibody, wherein the antibody targets P. gingivalis. In some alternatives, the antibody comprises at least one, two, three, four, five, or all six of the CDR regions of SEQ ID NOS: 1-2. In some alternatives, the antibody comprises a VH of a VH within SEQ ID NO: 1 and a VL of a VL within of SEQ ID NO: 2. In some alternatives, the antibody comprises at least one, two, three, four, five, or all six of the CDR regions of SEQ ID NOS: 1-2. In some alternatives, the antibody comprises CDR1 of SEQ ID NO:1. In some alternatives, the antibody comprises CDR2 of SEQ ID NO:1. In some alternatives, the antibody comprises CDR3 of SEQ ID NO:1. In some alternatives, the antibody comprises CDR4 of SEQ ID NO:2. In some alternatives, the antibody comprises CDR5 of SEQ ID NO: 2. In some alternatives, the antibody comprises CDR6 of SEQ ID NO:2. In some alternatives, the antibody comprises a VH of a VH within SEQ ID NO: 1 and a VL of a VL within of SEQ ID NO: 2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the histidine is present in the formulation at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the sucrose is present in the formulation at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the sucrose is present in the formulation at a concentration of about 85 mM. In some alternatives, the sucrose is present in the formulation at a concentration of about 150 mM. In some alternatives, the sucrose is present in the formulation at a concentration of about 200 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the Poloxamer P188 is present in the formulation at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is at a concentration of about 0.01% weight/volume.

Also disclosed herein is an antibody formulation comprising histidine, sucrose, Poloxamer P188, and an antibody, wherein the antibody comprises at least one, two, three, four, five, six, seven, or all eight of a VH segment that is at least 75%, 79%, or 80% identical to SEQ ID NO:1, a VL segment that is at least 75%, 79%, or 80% identical to SEQ ID NO: 2, a CDR1 that is at least 75%, 79%, or 80% identical to the CDR1 of SEQ ID NO:1, a CDR2 that is at least 75%, 79%, or 80% identical to the CDR2 of SEQ ID NO: 1, a CDR3 that is at least 75%, 79%, or 80% identical to the CDR3 of SEQ ID NO: 1, a CDR4 that is at least 75%, 79%, or 80% identical to the CDR4 of SEQ ID NO: 2, a CDR5 that is at least 75%, 79%, or 80% identical to the CDR5 of SEQ ID NO: 2, and/or a CDR6 that is at least 75%, 79%, or 80% identical to the CDR6 of SEQ ID NO: 2. In some alternatives, the antibody targets P. gingivalis. In some alternatives, the antibody comprises a VH of a VH within SEQ ID NO: 1 and a VL of a VL within of SEQ ID NO: 2. In some alternatives, the antibody comprises SEQ ID NO: 1. In some alternatives, the antibody comprises SEQ ID NO: 2. In some alternatives, the antibody comprises SEQ ID NO: 1 and SEQ ID NO: 2. In some alternatives, the antibody comprises a VH segment that is at least about 70%, at least about 75%, at least about 80%, at least about 85%, at least about 90%, at least about 95%, at least about 99%, and/or at least about 100% identical to SEQ ID NO:1. In some alternatives, the antibody comprises a VL segment that is at least about 70%, at least about 75%, at least about 80%, at least about 85%, at least about 90%, at least about 95%, at least about 99%, and/or at least about 100% identical to SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR1 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR2 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR3 of SEQ ID NO:1. In some alternatives, the antibody comprises a CDR segment that is at least about 70%, at least about 75%, at least about 80%, at least about 85%, at least about 90%, at least about 95%, at least about 99%, and/or at least about 100% identical to the CDR4 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR5 of SEQ ID NO:2. In some alternatives, the antibody comprises a CDR segment that is at least about 70%, at least about 75%, at least about 80%, at least about 85%, at least about 90%, at least about 95%, at least about 99%, and/or at least about 100% identical to the CDR6 of SEQ ID NO:2. In some alternatives, the antibody is present in the formulation at a concentration of about 0.01 mg/mL, about 0.05 mg/mL, about 0.1 mg/mL, about 0.5 mg/mL, about 0.9 mg/mL, about 0.1 mg/mL, about 0.12 mg/mL, about 0.15 mg/mL, about 0.2 mg/mL, about 0.5 mg/mL, about 1.0 mg/mL, about 5.0 mg/mL, about 7.5 mg/mL, about 10 mg/mL, about 15 mg/mL, about 20 mg/mL, or any concentration that in between. In some alternatives, the antibody is present in the formulation at a concentration of about 0.1 mg/mL. In some alternatives, the histidine is present in the formulation at a concentration of about 0.1 mM, about 1 mM, about 5 mM, about 10 mM, about 20 mM, about 40 mM, about 50 mM, about 100 mM, or any concentration in between. In some alternatives, the histidine is present in the formulation at a concentration of about 20 mM. In some alternatives, the sucrose is present in the formulation at a concentration of about 25 mM, about 50 mM, about 75 mM, about 85 mM, about 100 mM, about 125 mM, about 150 mM, about 175 mM, about 200 mM, about 250 mM, about 300 mM, about 400 mM, about 500 mM, or any concentration in between. In some alternatives, the sucrose is present in the formulation at a concentration of about 85 mM. In some alternatives, the sucrose is present in the formulation at a concentration of about 150 mM. In some alternatives, the sucrose is present in the formulation at a concentration of about 200 mM. In some alternatives, the formulation has a pH of about 3.0, about 3.5, about 4.0, about 4.5, about 5.0, about 6.0, about 7.0, about 8.0, about 8.5, about 9.0, or any pH in between. In some alternatives, the formulation has a pH of about 6.0. In some alternatives, the formulation has a pH of about 5.8. In some alternatives, the Poloxamer P188 is present in the formulation at a concentration of about 0.0001% weight/volume, about 0.0005% weight/volume, about 0.001% weight/volume, about 0.005% weight/volume, about 0.01% weight/volume, about 0.05% weight/volume, about 0.1% weight/volume, about 0.5% weight/volume, about 1% weight/volume, or any concentration in between. In some alternatives, the Poloxamer P188 is at a concentration of about 0.01% weight/volume.

In some alternatives, the antibody is an antigen binding molecule (ABM). In some alternatives, the antibody is KB001. In some alternatives, the antibody is at least about 75%, at least about 80%, at least about 85%, at least about 90%, at least about 95%, at least about 99%, and/or at least about 100% identical to KB001. In some alternatives, the antibody is KB001. In some alternatives, the antibody comprises a sequence that is at least 75%, 79%, or 80% identical to SEQ ID NO: 1. In some alternatives, the antibody comprises a sequence that is at least 75%, 79%, or 80% identical to SEQ ID NO: 2.

Also disclosed herein is a method of storing an antibody, the method comprising mixing the antibody with an antibody formulation that comprising any one of the formulations disclosed herein. In some alternatives, the antibody formulation is stored at room temperature (about 20-29° C.). In some alternatives, the antibody formulation is stored at the room temperature of about 25° C. In some alternatives, the antibody formulation is stored at below room temperature (<20° C.). In some alternatives, the antibody formulation is stored at a temperature between about 20° C. to about −80° C. In some alternatives, the antibody formulation is stored at about 5° C. In some alternatives, the antibody formulation is stored at above room temperature (>29° C.). In some alternatives, the antibody formulation is stored at a temperature between about 30 to about 80° C. In some alternatives, the antibody formulation is stored at about 40° C. In some alternatives, the antibody formulation is stored as a solid and/or frozen. In some alternatives, the antibody formulation is stored as a liquid.

Also disclosed herein is a use for any one of the antibody formulations disclosed herein as a storage formulation to enhance the stability of an at least one antibody or antigen binding molecule. In some alternatives, the antibody formulation is stored at room temperature (about 20-29° C.). In some alternatives, the antibody formulation is stored at the room temperature of about 25° C. In some alternatives, the antibody formulation is stored at below room temperature (<20° C.). In some alternatives, the antibody formulation is stored at a temperature between about 20° C. to about −80° C. In some alternatives, the antibody formulation is stored at about 5° C. In some alternatives, the antibody formulation is stored at above room temperature (>29° C.). In some alternatives, the antibody formulation is stored at a temperature between about 30 to about 80° C. In some alternatives, the antibody formulation is stored at about 40° C. In some alternatives, the antibody formulation is stored as a solid and/or frozen. In some alternatives, the antibody formulation is stored as a liquid.

The present disclosure also relates to the formulation for antigen-binding molecules, e.g., biomolecules, such as antibodies, that bind to Porphyromonas gingivalis, and the treatment and/or prevention of systemic diseases associated with chronic inflammation, multi-systems inflammation, and/or periodontal disease(s) associated with P. gingivalis infection and/or the continuous release of exo-toxins therefrom, using such P. gingivalis bacteria and exotoxin antigen-binding molecules, e.g., biomolecules. Periodontal disease, including Porphyromonas gingivalis infection, has been implicated in various conditions, disorders or diseases including, without limitation, vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); and longevity or age-related disorder, regenerative and stem cell dysfunction.

As disclosed herein, the antibodies of the present application are clinically validated for eliminating P. gingivalis.

In some embodiments, the ABM is an antibody. For instance, the antibody KB-001 is a monoclonal antibody with unique binding to P. gingivalis and its virulence factors, In some embodiments, the ABM binds to an epitope comprising GVSPKVCKDVTVEGSNEFAPVQNLT (SEQ ID NO: 4) and/or YCVEVKYTAGVSPK (SEQ ID NO: 5) and/or YTYTVYRDGTKIK (SEQ ID NO: 3) found in the HagA repeat epitope hemagglutinin/gingipains/adhesin domain (HXHRE domain).

In some embodiments, the antibody and/or ABM of the formulation binds to an epitope that includes a “Hag x repeat” section, which is a motif that is present in various proteins/peptides of interest for gingipains. The motif comprises: YTYTVYRDGTKIK (SEQ ID NO: 3) as a component of the epitope for KB001. The motif is present at least once in Pg, but in pre-processed forms of the protein, can be present multiple times (e.g., 2, 3, 4, 5, 6, 7, 8, 9 or 10, 11, 12, 13, 14, 15 times or more for various complexes). By using antibodies that target to this motif, numerous antibodies can bind to the target of interest in an enhanced manner. The motif can comprise longer sequences as well, such as: YTYTVYRDGTKIK (SEQ ID NO: 3). Depending on Pg strain this motif is repeated at least twice on Kgp, 3× on RgpA and up to 6× on HagA. In some embodiments, the epitope occurs at least 10 times on proteins associated with the Pg cell surface, making it superior for therapeutics.

In some embodiments, the antibody and/or ABM comprises SEQ ID NO: 1. In some embodiments, the ABM comprises an amino acid sequence that is at least about 70%, at least about 75%, at least about 80%, at least about 85%, at least about 90%, at least about 95%, at least about 99%, and/or at least about 100% identical to SEQ ID NO: 1. In some embodiments, the ABM comprises SEQ ID NO: 2. In some embodiments, the ABM comprises an amino acid sequence that is at least about 70%, at least about 75%, at least about 80%, at least about 85%, at least about 90%, at least about 95%, at least about 99%, and/or at least about 100% identical to SEQ ID NO: 2. In some embodiments, the ABM comprises SEQ ID NO: 1 and SEQ ID NO: 2. In some embodiments, the ABM comprises an amino acid sequence that is at least about 70%, at least about 75%, at least about 80%, at least about 85%, at least about 90%, at least about 95%, at least about 99%, and/or at least about 100% identical to SEQ ID NO: 1, and an amino acid sequence that is at least about 70%, at least about 75%, at least about 80%, at least about 85%, at least about 90%, at least about 95%, at least about 99%, and/or at least about 100% identical to SEQ ID NO: 2.

Any of the embodiments provided herein can be directed to or substituted with ABM (including antibodies) that bind to the following sequence: YTYTVYRDGTKIK (SEQ ID NO: 3).

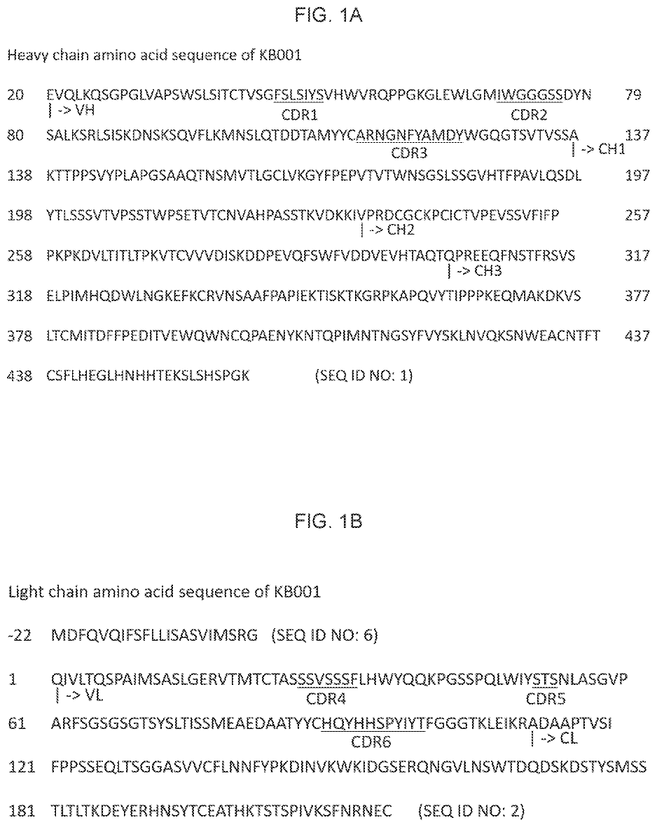

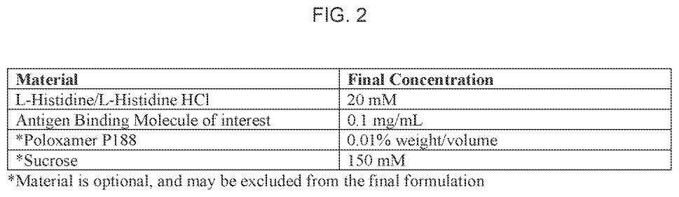

It will be understood that the formulation of the present application may be suitable for the storage of any antibody and/or protein. Consequently, it is envisioned that the formulation of the present application may be used for the storage of any protein or antibody beyond those that target P. gingivalis, and may also be used for the storage of a mixture of antibodies, antigen binding molecules, and/or proteins as disclosed herein. In some embodiments, the formulation comprises SEQ ID NO: 1 and SEQ ID NO:2 from FIG. 1 as the antibody, and a formulation as set forth in FIG. 2 (e.g., 20 mM L-histidine/L-histidine HCl, 0.1 mg/mL of the antibody, and optionally one or both of poloxamer P188 (at 0.01% weight/volume) and sucrose (at 150 mM).

P. gingivalis

Porphyromonas gingivalis is a keystone pathogen that converts the local and distant healthy microbiome of an individual into a disease-forming biofilm of both the mouth and gut. P. gingivalis has multiple survival mechanism, which creates a grossly undiagnosed chronic active/inactive infection in the host leading to a “silent” chronic state of systemic and end organ inflammation and ultimate failure.

Pg can recolonize one week after regular dental cleaning most likely from small remaining islands of viable bacteria in subgingival biofilm missed and/or resistant to the specific procedure and re-establishes its life-long bio-film 30 days after non-surgical periodontal treatment. It can even be present in a visually clean and healthy-looking mouth. This leads to a slow, low to high level of local and systematic damage that is mostly symptomless (clinically silent) and often without a person even knowing they have the infection. In some embodiments, KB-001 prevents Pg from synthesizing its secreted outer membrane vesicles (OMVs) containing virulence protein complexes, resulting in the bacteria shutting down its metabolic and host defense functions. In some embodiments, KB-001 has the capability to treat Pg, eliminating it and all of its virulence factors.

The pathogen hypothesis for Alzheimer's disease has been met with new attention over the last 5 years, but the push back has been the Immune Privilege of the Brain and whether the suspected pathogen source is local or peripheral to the brain tissues. As disclosed herein, the inventors show that the effect of P. gingivalis in the brain is mostly if not entirely from an oral peripheral source. Second, the inventors have generated new data from the largest analysis of AD brain tissues to date showing no presence of P. gingivalis DNA in the brain. Thirdly, the inventors have identified and discovered a one-of-a-kind virulent subunit of the primary suspected pathogen in the strategic sites of AD brain tissues. It is a unique subunit toxin “XXX Epitope” domain of P. gingivalis. This virulent subunit toxin plays a massive role in disrupting the NLRP3 inflammasome and the IL-1b pathways. IL-1b and ubiquinone have been shown to trigger the pathogenesis and progression of Alzheimer's disease. This same virulent subunit toxin plays an equally large role in systemic inflammation, immune disruption, and has disease-causing effects on basic human cellular biology. The delivery of the virulent toxin to the brain appears to be primarily vascular, with possibly additional access through neuronal, all however, occurring from the oral source of P. gingivalis. The data described herein strongly suggests for the first time that the “XXX Epitope” and related material are coming to the brain in AD as secreted by outer membrane vesicles from the bacterial surface of oral cavities. Further research is currently being conducted by the inventors into the prevalence of, genotypes of, and relative amounts of the presence of P.g. and its associated secreted exotoxins (OMVs-gingipains and LPS) and anti-P.g./LPS antibodies in patients with increased markers of systemic vascular inflammation and overexpression of inflammasome pathways, as well as the prevalence of increased markers of vascular and gut inflammation in patients with and without P.g. infection.

### Definitions

As used herein, the term “antigen binding molecule” (ABM) refers to a polypeptide that includes one or more fragments of an antibody that retain the ability to specifically bind to an antigen, e.g., bacterial antigen (e.g., gingipain, adhesin hemagglutinin complex). ABM encompasses antigen-binding fragments of antibodies (e.g., single chain antibodies, Fab and Fab fragments, F(ab′)₂, Fd fragments, Fv fragments, scFv, and domain antibodies (dAb) fragments (e.g., nanobodies) (see, e.g. de Wildt et al., Eur J. Immunol. 1996; 26 (3): 629-39; which is incorporated by reference herein in its entirety)) as well as complete antibodies. An ABM can include an antibody or a polypeptide containing an antigen-binding domain of an antibody. In some embodiments, an ABM can include a monoclonal antibody or a polypeptide containing an antigen-binding domain of a monoclonal antibody. For example, an ABM, e.g., antibody, can include a heavy (H) chain variable region (abbreviated herein as VH), and/or a light (L) chain variable region (abbreviated herein as VL). In another example, an ABM, e.g., antibody, includes two heavy (H) chain variable regions and/or two light (L) chain variable regions. An ABM, e.g., antibody, can have the structural features of IgA, IgG, IgE, IgD, IgM (as well as subtypes and combinations thereof). An ABM, e.g., antibody, can be from any source, including mouse, rabbit, pig, rat, and primate (human and non-human primate) and primatized (e.g., humanized) antibodies. ABM also include mini-bodies, humanized antibodies, chimeric antibodies, and the like, as well as nanobodies (single variable domain with two constant heavy domains) derived from Camelidae (camels and llamas) family. In addition they can be synthesized using protein synthetic chemistries ab initio.

As used herein an “antibody” refers to any immunoglobulin (Ig) molecule comprised of four polypeptide chains, two heavy (H) chains and two light (L) chains, interconnected by disulfide bonds or any functional fragment, mutant, variant, or derivation thereof, which retains the essential epitope binding features of an Ig molecule. The heavy chain constant region can include CH1, hinge, CH2, CH3, and, sometimes, CH4 regions. In some embodiments, for therapeutic purposes, the CH2 domain can be deleted or omitted. “Antibody” also refers to IgG, IgM, IgA, IgD or IgE molecules or antigen-specific antibody fragments thereof (including, but not limited to, a Fab, F(ab′)₂, Fv, disulfide linked Fv, scFv, single domain antibody, closed conformation multi-specific antibody, disulfide-linked scFv, diabody), whether derived from any species that naturally produces an antibody, or created by recombinant DNA technology; whether isolated from serum, B-cells, hybridomas, transfectomas, yeast or bacteria.

The VH and VL regions can be further subdivided into regions of hypervariability, termed “complementarity determining regions” (“CDR”), interspersed with regions that are more conserved, termed “framework regions” (“FR”). The extent of the framework region and CDRs has been defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4. In some embodiments, an ABM, e.g., antibody, includes 1, 2, 3, 4, 5, and/or 6 CDRs.

The terms “antigen-binding fragment” or “antigen-binding domain,” which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, the two domains of the Fv fragment, VL and VH, can be joined, using recombinant methods, by a synthetic linker that enables them to be made as a single protein chain in which the VL and VH regions pair form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique.

The term “Fc region” refers to the C-terminal region of an immunoglobulin heavy chain, which may be generated by papain digestion of an intact antibody. The Fe region may be a native sequence Fc region or a variant Fc region. The Fc region of an immunoglobulin generally comprises two constant domains, a CH2 domain and a CH3 domain, and optionally comprises a CH4 domain. Specifically, in IgG, IgA and IgD types, the Fc region is composed of two identical protein fragments derived from CH2 and CH3 of the heavy chains. Fc regions of IgM and IgE contain three heavy chain constant domains, CH2, CH3, and CH4.

The term “human antibody” or “human ABM” includes antibodies or ABMs having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242) or Chothia, C. et al. (1987) J. Mol. Biol. 196:901-917; which are incorporated by reference herein in their entireties. The human antibodies or ABMs of the present disclosure may include amino acid residues not encoded by human germline immunoglobulin sequences (e.g., mutations introduced by random or site-specific mutagenesis in vitro or by somatic mutation in vivo), for example in the CDRs. Any suitable method for generating human or fully human antibodies or ABMs can be used, including but not limited to, EBV transformation of human B cells, selection of human or fully human antibodies from antibody libraries prepared by phage display, yeast display, mRNA display or other display technologies, and also from mice or other species that are transgenic for all or part of the human Ig locus comprising all or part of the heavy and light chain genomic regions defined further above. Selected human antibodies or ABMs may be affinity matured by art recognized methods including in vitro mutagenesis, preferably of CDR regions or adjacent residues, to enhance affinity for the intended target.

By “humanized antibody” or “humanized ABM” is meant an antibody or ABM that is composed partially or fully of amino acid sequences derived from a human antibody germline by altering the sequence of an antibody having non-human complementarity determining regions (CDR). A humanized antibody or ABM can include an antibody or ABM that comprises heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which non-human CDR sequences are introduced into human VH and VL sequences to replace the corresponding human CDR sequences. Also a “humanized antibody” is an antibody or a variant, derivative, analog or fragment thereof that specifically binds to an antigen of interest and which comprises a framework (FR) region having substantially the amino acid sequence of a human antibody and a CDR having substantially the amino acid sequence of a non-human antibody.

The term “chimeric antibody” refers to an antibody that comprises heavy and light chain variable region sequences from one species (e.g., mouse) and constant region sequences from another species (e.g., human), such as antibodies having murine heavy and light chain variable regions linked to human constant regions.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives to hybridoma-based production systems.

As used herein, an “epitope” can be formed both from contiguous amino acids, or noncontiguous amino acids juxtaposed by folding of a protein. Epitopes formed from contiguous amino acids are typically retained on exposure to denaturing solvents, whereas epitopes formed by folding are typically lost on treatment with denaturing solvents. An epitope includes the unit of structure specifically bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In some embodiments, the epitope may have both linear and conformational sequence determinants and thus be derived from a single monomer, homo-dimer, homo trimer, etc., and/or hetero-dimers, hetero-trimers, etc.

The term “compete” as used herein in the context of antigen binding molecules (e.g., antibodies or antigen-binding fragments thereof) that compete for the same binding target, antigen, or epitope refers to competition between antigen binding molecules as determined by an assay in which the antigen binding molecule (e.g., antibody or immunologically functional fragment thereof) being tested prevents or inhibits (e.g., reduces) specific binding of a reference antigen binding molecule (e.g., a reference antibody) to a common antigen (e.g., P. gingivalis gingipain or a fragment thereof). Any suitable competitive binding assay can be used to determine if one antigen binding molecule competes with another, for example: solid phase direct or indirect radioimmunoassay (MA), solid phase direct or indirect enzyme immunoassay (EIA), sandwich competition assay, solid phase direct labeled assay, solid phase direct labeled sandwich assay, solid phase direct label MA using I-125 label, solid phase direct biotin-avidin EIA, and direct labeled MA. Typically, such an assay involves the use of purified antigen bound to a solid surface or cells bearing either of these, an unlabeled test antigen binding protein and a labeled reference antigen binding molecule. Competitive inhibition is measured by determining the amount of label bound to the solid surface or cells in the presence of the test antigen binding molecule. Usually the test antigen binding protein is present in excess. Antigen binding proteins identified by competition assay (competing antigen binding molecules) include antigen binding molecules binding to the same epitope as the reference antigen binding molecules and antigen binding molecules binding to an adjacent epitope sufficiently proximal to the epitope bound by the reference antigen binding molecule for steric hindrance to occur. Usually, when a competing antigen binding molecule is present in excess, it will inhibit (e.g., reduce) specific binding of a reference antigen binding molecule to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologues, paralogs, fragments and other equivalents, variants, fragments, and analogs of the foregoing.

Amino acid substitutions in a native protein sequence may be “conservative” or “non-conservative” and such substituted amino acid residues may or may not be one encoded by the genetic code. A “conservative amino acid substitution” is one in which the amino acid residue is replaced with an amino acid residue having a chemically similar side chain (i.e., replacing an amino acid possessing a basic side chain with another amino acid with a basic side chain). A “non-conservative amino acid substitution” is one in which the amino acid residue is replaced with an amino acid residue having a chemically different side chain (i.e., replacing an amino acid having a basic side chain with an amino acid having an aromatic side chain). The standard twenty amino acid “alphabet” is divided into chemical families based on chemical properties of their side chains. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and side chains having aromatic groups (e.g., tyrosine, phenylalanine, tryptophan, histidine).

As used herein, “sequence identity” or “identity” in the context of two amino acid or nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Any suitable means for making this adjustment may be used. This may involve scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage can be calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Any suitable methods of alignment of sequences for comparison may be employed. Thus, the determination of percent identity between any two sequences can be accomplished using a mathematical algorithm. Preferred, non-limiting examples of such mathematical algorithms are the algorithm of Myers and Miller, CABIOS, 4:11 (1988), which is hereby incorporated by reference in its entirety; the local homology algorithm of Smith et al, Adv. Appl. Math., 2:482 (1981), which is hereby incorporated by reference in its entirety; the homology alignment algorithm of Needleman and Wunsch, JMB, 48:443 (1970), which is hereby incorporated by reference in its entirety; the search-for-similarity-method of Pearson and Lipman, Proc. Natl. Acad. Sci. USA, 85:2444 (1988), which is hereby incorporated by reference in its entirety; the algorithm of Karlin and Altschul, Proc. Natl. Acad. Sci. USA, 87:2264 (1990), which is hereby incorporated by reference in its entirety; modified as in Karlin and Altschul, Proc. Natl. Acad Sci. USA, 90:5873 (1993), which is hereby incorporated by reference in its entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988), Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994), which are hereby incorporated by reference in their entirety. The ALIGN program is based on the algorithm of Myers and Miller, supra. The BLAST programs of Altschul et al., JMB, 215:403 (1990); Nucl. Acids Res., 25:3389 (1990), which are hereby incorporated by reference in their entirety, are based on the algorithm of Karlin and Altschul supra.

As used herein, the terms “treat,” “treatment,” “treating,” or “amelioration” refer to therapeutic treatments, wherein the object is to reverse, alleviate, ameliorate, inhibit, slow down or stop the progression or severity of a condition, e.g., a chronic inflammatory condition, associated with a disease or disorder, e.g. arteriosclerosis, gingivitis, etc. The term “treating” includes reducing or alleviating at least one adverse effect or symptom of a condition, disease or disorder associated with, e.g., arteriosclerosis, gingivitis, etc. Treatment is generally “effective” if one or more local or systemic conditions, symptoms or clinical biomarkers of disease are reduced. Alternatively, treatment is “effective” if the progression of a disease is reduced or halted. That is, “treatment” includes not just the improvement of symptoms or biomarkers, but also a cessation of, or at least slowing of, progress or worsening of symptoms compared to what would be expected in the absence of treatment. Thus, a treatment is considered effective if one or more of the signs or symptoms of a condition described herein are altered in a beneficial manner, other clinically accepted symptoms are improved, or even ameliorated and/or reversed back to a more normal or normal state, or a desired response is induced e.g., by at least 10% following treatment according to the methods described herein. Beneficial or desired clinical results include, but are not limited to, alleviation of one or more symptom(s), diminishment of extent of disease, e.g., chronic inflammatory disease, stabilized (e.g., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The terms “buffer” and “formulation” can be used interchangeably herein, unless otherwise clearly denoted by the context of the statement.

Efficacy of an agent, e.g., ABM, can be determined by assessing physical indicators of a condition or desired response, e.g. inflammation and/or infection. Efficacy can be assessed in animal models of a condition described herein, for example treatment of systemic chronic inflammatory diseases associated with an oral infection, e.g., periodontal disease. When using an experimental animal model, efficacy of treatment is evidenced when a statistically significant change occurs in one of a number of criteria, including a one or more biomarkers associated with inflammation following infection. In some embodiments, treatment according to the methods described herein can reduce the levels, and/or eliminate and/or prevent the colonization of the disease causing bacteria Porphyromonas gingivalis. In some embodiments, treatment according to the methods described herein can reduce the levels of a biomarker(s) or symptom(s) or the tissue pathology of a condition, e.g. infection or recolonization by at least 10%, at least 15%, at least 20%, at least 25%, at least 30%, at least 40%, at least 50%, at least 60%, at least 70%, at least 80%, at least 90% or more, at least 95% or more, at least 98% or more, at least 99% or more, or by about 100%.

The term “effective amount” as used herein refers to the amount of an active agent, e.g., ABM, or composition needed to alleviate at least one or more criteria listed above of the disease or disorder, and relates to a sufficient amount of active agent or pharmacological composition to provide the desired effect. The term “therapeutically effective amount” therefore refers to an amount of active agent or composition that is sufficient to provide a particular anti-bacterial or anti-recolonization effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease.

As used herein, “subject” means a human or animal. The animal can be a vertebrate, including a mammal, such as a primate, dog or rodent. Primates include human, chimpanzees, cynomolgus monkeys, spider monkeys, and macaques, e.g., Rhesus. Rodents include mice, rats, woodchucks, ferrets, rabbits and hamsters. Animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a primate, e.g., a human. The terms, “individual,” “patient” and “subject” are used interchangeably herein.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. Delivery and/or placement options include any suitable medicament delivery systems for intraoral, interproximal, intrasulcular, intra-periodontal pocket, intracanal, and intranasal. In some embodiments, a suitable delivery option includes any suitable mechanical and automated dental and medical syringes, including all calibrated and non-calibrated, all attachments, and all designs of tips including but not limited to blunt ended, and side port; Medicament delivery trays and systems including PerioProtect Trays; Medicament applicator delivery systems; Slow releasing medical preparation for intrasulcular drug delivery; Filler, oral packing, fiber, microparticles, films, gels, injectable gels, vesicular systems, strips compacts, chip, hydrogel, thermal gel, liquid, solid, including Actisite, Arestin, Atridox, Ossix Plus, Periochip, Periostat, Periofil; Injectable systems; Professional irrigation systems including piezoelectric and ultrasonic cavitron units with and without reservoir including Ora-Tec Viajet and Oral irrigation systems including Interplak, Waterpik, Hydrofloss, Viajet, Airfloss and Pro.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is used herein to indicate a non-limiting example. Thus, “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10:0763766321); Kendrew et al. (eds), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

### Associated Diseases

In some embodiments, the formulation of the present disclosure is used to enhance the storage life and/or stability of an antibody and/or ABM that binds to P. gingivalis. It will be understood by one skilled in the art that the consequent administration of the antibody and/or ABM to a patient infected with P. gingivalis may have a therapeutic effect. As a non-limiting example, when topically applied via a solution to the infected gums of patients with P. gingivalis binds specifically to the bacterial outer membrane surface, e.g., the molecular complex in the outer- and inner-membranes of the secreted vesicles (exomes) containing complex of toxins (LPS), gingipain proteases, and hemagglutinin. In some embodiments, the formulation binds to a repeating epitope present on multiple localities of the pre- and post-processed hetero-dimer/trimer. In some embodiments, the ABM find use in a prolonged topical oral setting, or intravenous, subcutaneous, intradermal, nebulized or intra-thecal administration. Without being bound to theory, P. gingivalis is thought to relocate into various other tissues/organs/end capillary beds throughout the body and cause local inflammation at these sites. In some embodiments, delivering a formulation of the present disclosure to local or primary site of infection (e.g., oral or subgingival infection) addresses the systemic infection or distant infections at one or more secondary sites. In some embodiments, a formulation that is a nanobody allows for deeper tissue penetration, e.g., to treat various P. gingivalis related cancers.

In some embodiments, the antibody and/or ABM of the present disclosure can be effective in preventing the periodontal growth or recolonization by P. gingivalis in a subject to which the formulation is administered. Without being bound to theory, the ABM, e.g., antibody, can bind to critical survival surface structures of the bacteria so as to interfere with the bacteria's ability to attach, stay attached to form a protective bio-film, derive metabolites/energy sources, and inactivate anti-bacterial defenses and thus survive. This can cause the bacteria to die and can destroy its biofilm, such destruction of the biofilm changing the nutrient support to other dysbiotic bacteria that may have formed around and have inter-dependence with P. gingivalis colonies. As a result, the bacterial molecules leading to active chronic inflammation and disease e.g. gingipains/LPS are no longer produced, thus reducing and/or eliminating local/systemic inflammation in the human host, leading to repair, healing and re-establishment of a more healthy oral microbiome.

A variety of conditions, disorders or diseases may be treated through the use of a formulation of the present disclosure. Without being limited by theory, the use of the ABM in the formulation of the present disclosure to eliminate and/or prevent recolonization of P. gingivalis in the sub-gingival gum line can in some embodiments interrupt and/or block, or over express the host's inflammatory pathways, such as the inflammasome NLRP3/Interleukin-1β/IL-6 pathways, AIM2, C-reactive protein, the PCSK9 pathway, and the Interleukin-1β innate immunity pathway. In addition, the local and systemic secretion by the bacteria of tissue-damaging outer-membrane vesicles containing a potent mixture of toxins can be curtailed. The formulation of the present disclosure can, in certain embodiments, allow for specifically and locally targeting the P. gingivalis oral infection, which can be the root cause of a chronic active inflammation and toxemia throughout the host's body. In some embodiments, use of the formulation to specifically target and eliminate the disease-causing bacterial source, while sparing other existing oral bacterial strains, provides for treatment of the systemic inflammation without interrupting the complex host inflammation pathways. In some embodiments, use of the ABM in the formulation as disclosed herein avoids or reduces local and/or systemic side effects that may result from intervening in the disrupting/reducing/overexpressing inflammatory pathways such as but not limited to inflammasome NLRP3/Interleukin-1β/IL-6 pathways, C-reactive protein, the PCSK9 pathway, and the Interleukin-1β innate immunity pathway for treating a disease.

In some embodiments, P. gingivalis infection occurs in the mouth, gum, teeth, oral cavity, brain, across the blood brain barrier, gut, blood, bone, and/or soft tissues. In some embodiments, P. gingivalis infection occurs in multiple organs. In some embodiments, P. gingivalis infection is local. In some embodiments, P. gingivalis infection is systemic. In some embodiments, P. gingivalis infection is one of several infections in a subject; non-limiting examples of which include Helicobacter pylori, Adenovirus, Acinetobacter spp., Actinomyces spp., Aeromonas hydrophila, Aggregatibacter actinomycetemcomitans, Ascaris lumbricoides, Astrovirus, Bacillus spp., Bacillus cereus, Bifidobacterium spp., Camplylobacter spp., Campylobacter jejuni, Camplylobacter rectus, Candida albicans, Chlamydia trachomatis, Chlamydophila pneumoniae, Clostridium spp., Clostridium botulinum, Clostridium difficile, Clostridium perfringens, Clostridium tetanus, Coronaviridaea, Corynebacterium diphtheriae, Cryptococcus neoformans, Cryptosporidium parvum, Cyclospora cayetanensis, Eikenella corrodens, Entamoeba histolytica, Enterobacteriaceae spp., Enterobius vermicularis, Enterovirus, Escherichia coli, Eubacterium nodatum, Fusobacterium spp., Fusobacterium nucleatum, Giardia lamblia, Haemophilus influenzae, Hepatitis, Hymenolepis nana, Influenza, Klebsiella spp., Klebsiella pneumoniae, Lactobacillus casei, Listeria monocytogenes, Moraxella spp., Moraxella catarrhalis, Mycobacterium tuberculosis, Mycoplasma pneumoniae, Necator americanus, Neisseria gonorrhoeae, Neisseria meningitidis, Norovirus, Parviomonas micra, Pasteurella multocida. Peptostreptococcus, Prevotella intermedia, Prevotella nigrescens, Propionibacterium acne, Proteus mirabilis, Pseudomonas aeruginosa, Rotavirus, Salmonella typhi, Salmonella typhimurium, Serratia marcescens, Shigella dysenteriae. Shigella flexneri, Shigella sonnei, Staphylococcus aureus, Staphylococcus epidermidis, Streptococcus spp., Streptococcus agalactiae, Streptococcus enterococci, Streptococcus gordonii, Streptococcus intermedius, Streptococcus mitis, Streptococcus mutans, Streptococcus oralis, Streptococcus pneumoniae, Streptococcus pyogenes, Streptococcus sanquinis, Streptococcus sobrinus, Streptococcus viridans, Strongyloides stercoralis, Taenia saginata, Taenia solium, Tannerella forsythia, Treponema denticola, Vibrio cholerae, and Yersinia enterocolitica. In some embodiments, the at least one additional infection is bacterial, viral, and/or parasite. In some embodiments, the multiple infections form a community biofilm. These biofilms may form a combination of virulence factors, any of which may be targeted as part of subsequent treatment. In some embodiments, virulence factors from P. gingivalis may be targeted as part of treatment or therapy.

In some embodiments, a P. gingivalis infection at an oral site affects end organs, such as, without limitation, large and small vessels of the heart, carotid arteries, vessels in the brain, liver, joints, lungs, pancreas, reproductive system. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g., Parkinson's), Peri-Implantitis, periodontal disease and/or associated bone loss, cognitive disorders (e.g., early middle late dementia Alzheimer's disease); regenerative and stem cell dysfunction; and age-related disorder.

In some embodiments, the condition, disorder, disease, or complication is present in a single cell, organ, tissue, or organ system. In some embodiments, the condition, disorder, disease, or complication is present in multiple cells, organs, tissues, or organ systems.

As disclosed herein, there are many phenotypes that may occur during P. gingivalis infection. Non-limiting examples include an increase in CRISPR-Cas gene expression at the site of infection, an increase in local or systemic inflammation, an increase in the biofilm and/or presence of P. gingivalis, an increase in the activity or activation of inflammasomes, the diversion of oxygen, iron, and other nutrients to P. gingivalis, an increase in cytokine levels, increased host cell death, an increase in systemic inflammation, change of P. gingivalis protein expression, increased proinflammatory mediators, and enhanced chronic distant site inflammatory atherosclerosis. Subsequently, treatment by used of the present formulations may inhibit, reduce, or eliminate any or multiple of the above phenotypes. In some embodiments, the P. gingivalis infection is in the mouth, gums, brain, gut/gastrointestinal system, blood brain barrier, bone, plasma/blood, soft tissue, or any combination thereof. In some embodiments, targeting the P. gingivalis infection further comprises administration of a small molecule, antibiotic, or drug affective against P. gingivalis. This will be understood to include any effective medicant that acts against P. gingivalis, including small molecules, antibiotics, or drugs that target P. gingivalis virulence factors, increases the production of proteases targeting P. gingivalis, reduces P. gingivalis oxygen, iron, and/or other nutrient uptake, alters protein production in P. gingivalis, alters bacterial metabolism, and/or enhances cell death for P. gingivalis.

Conditions, disorders or diseases treated by administration of a formulation of the present disclosure includes, without limitation, vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral squamous carcinomas, gastrointestinal cancer, pancreatic cancer, lung cancer, etc); gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); cognitive disorder (e.g., Alzheimer's disease); neuroinflammatory diseases; and longevity and/or age-related disorders. In general terms, the method includes identifying a subject in need of treating a condition, disorder or disease, as disclosed herein, and administering to the subject a therapeutically effective amount of the formulation of the present disclosure, to thereby treat the condition, disorder or disease.

In some embodiments, the condition, disorder or disease is a vascular disease. A variety of vascular diseases can be treated by use of the present formulations. In some embodiments, the vascular disease is, without limitation, cardiovascular disease, atherosclerosis, coronary artery disease, myocardial infarction, stroke, or cardiac hypertrophy. Without being bound by theory, P. gingivalis and its virulence factors (e.g., outer membrane vesicles (OMVs), LPS, peptidylarginine deiminase (PPAD), gingipains, hemagglutinins, and fimbriae) are thought to disrupt the inflammatory pathways of heart and systemic vascular disease (CVD/Stroke), including the NLRP3/Interleukin-1β/IL-6 pathways, C-reactive protein (CRP) elevation, the PCSK9 pathway, and the suppression of adaptive immunity via reduction of regulatory T cells (Tregs). P. gingivalis infection can be associated with an increased risk of heart attack, and P. gingivalis is involved with forming oxidized LDL taken up by macrophages, leading to foam cell formation. These atherosclerotic lesions can develop a necrotic core, often forming a thrombus, leading to a downstream event (i.e. heart attack, stroke) Periodontal disease and/or P. gingivalis can be associated with elevated levels of systemic inflammatory markers, such as CRP, IL-6, and Lp-PLA2, Hb-A1c, IL-1b. P. gingivalis can play a major role in Abdominal Aortic Aneurysm development and salivary MPO enzyme activity. Periodontal therapy, as an intervention for improved oral health, can facilitate the management of thrombotic risk, and in the long term can contribute to the prevention of cardiovascular events in patients at risk.

In some cases, the development of atherosclerosis is due to systemic inflammation caused by severe periodontitis. Without being bound by theory, systemic inflammation induced by severe periodontitis, such as those associated with enhanced the secretion of pro-inflammatory cytokines from macrophages and increased the adhesion of monocytes to endothelial cells induce by P. gingivalis LPS, can exacerbate atherosclerosis via, in part, causing aberrant functions of vascular endothelial cells and the activation of macrophages. Further, patients with periodontitis can show higher serum pro-inflammatory cytokines such as tumor necrosis factor (TNF)-α, interleukin (IL)-1β, or IL-6. P. gingivalis can alter genes responsible for mitochondrial function and downregulate gene expression in the signaling pathway, which can lead to mitochondrial dysfunction and metabolic imbalance that promote the development of atherosclerosis. In some embodiments, P. gingivalis can prevent the regression of atherosclerotic plaques by interfering with reverse cholesterol transport. P. gingivalis can also promote atherosclerosis through alteration of gut microbiota, increased IL-1β, IL-18, and TNF-α production in peritoneal macrophages and gingival or aortic gene expression of the NOD-like receptor family, NLRP3, IL-1β, pro-IL-18 and pro-caspase-1, activation of the NLRP3 inflammasome, e.g., through CD36/SR-B2 and TLR2.

Chronic periodontitis (CP) can be associated with increased serum levels of HDL, Ox-LDL, hs-CRP, Hb-A1c, Lp-PLA2, MPO, LDH, troponins T & I, NT pro-BNP, and P selectin. Further, infection of type II P. gingivalis can cause prolonged cytokine response such as IL-1β, IL-8 and TNFα. Elevated cardiac markers found in periodontitis patients indicates that they may carry potential risks in developing cardiac lesions.

In some cases, P. gingivalis contribute to endothelial dysfunction and/or atherosclerotic cardiovascular disease. Without being limited by theory, P. gingivalis may cause vascular damage and increased endothelial permeability by degrading, via gingipain proteases, platelet endothelial cell adhesion molecule-1, and vascular endothelial cadherin, which play a role in endothelial junctional integrity. The vascular damage can increase endothelial permeability and initiate several processes implicated in atherosclerosis, including platelet aggregation, induction of proinflammatory cytokine release, and promotion of leukocyte extravasation to subendothelial regions.

Further, P. gingivalis promotes cardiac rupture after myocardial infarction (MI). Without being bound by theory, P. gingivalis is thought to invade the ischemic myocardium, promote cardiomyocyte apoptosis through activation of p18 Bax by gingipain, increase oxidative stress and MMP-9 protein level and activity, causing cardiac rupture. P. gingivalis-secreted factors can also promote cardiac hypertrophy, through activation of MEK/ERK signal pathways, Toll-like receptor-2 signaling. In some cases, mitogen-activated protein kinase kinase is involved in P. gingivalis-induced myocardial cell hypertrophy and apoptosis. In some cases, components of P. gingivalis spent culture medium increases total MEK-1 and ERK-1 protein products, but also causes increased cellular size, DNA fragmentation, and nuclear condensation in H9c2 cells. These three parameters, and the phosphorylated ERK-1 protein products of H9c2 cells treated with P. gingivalis medium, can be significantly reduced after pre-administration of U0126. The results indicate that P. gingivalis-secreted factors may initiate MEK/ERK signal pathways and lead to myocardial cell hypertrophy and apoptosis.

In some cases, P. gingivalis induces myocardial hypertrophy through Toll-like receptor-2 signaling in the isoproterenol-induced myocardial hypertrophy model. Regulation of chronic inflammation induced by periodontitis may have a key role in the treatment of myocardial hypertrophy. In some embodiments, P. gingivalis enhances myocardial vulnerability, thereby promoting post-infarct cardiac rupture. In some embodiments, Infection with Porphyromonas gingivalis (P.g.) promotes cardiac rupture after MI; P.g. invades the ischemic myocardium; Infection with P.g. promotes the accumulation of p18 Bax; Gingipains from P.g. activate Bax and promote cardiomyocyte apoptosis; Infection with P.g. promotes oxidative stress and MMP-9 protein level and activity.

In some embodiments, infection with periodontal pathogens can cause an adverse outcome after myocardial infarction (MI). C57BL/6J mice were inoculated with Porphyromonas gingivalis (P.g.), a major periodontal pathogen, or injected with phosphate-buffered saline (PBS) into a subcutaneously-implanted steelcoil chamber before and after coronary artery ligation. A significant increase in mortality, due to cardiac rupture, was observed in the P.g.-inoculated MI mice Ultrastructural examinations revealed that P.g. invaded the ischemic myocardium of the P.g.-inoculated MI mice. The expression of p18 Bax, an active form of pro-apoptotic Bax protein, markedly increased in the P.g.-inoculated MI hearts. In vitro experiments demonstrated that gingipain, a protease uniquely secreted from P.g., cleaved wild type Bax at Arg34, as evidenced by the observation that the cleavage of Bax by gingipain was completely abolished by the Arg34Ala mutation in Bax. Treatment with immunoglobulin Y against gingipain significantly decreased the mortality of the P.g.-inoculated MI mice caused by cardiac rupture. Furthermore, inoculation of P.g. also resulted in an increase of MMP-9 activity in the post-MI myocardium by enhancing oxidative stress, possibly through impairing the selective autophagy-mediated clearance of damaged mitochondria. Without being bound by theory, infection with P.g. during MI can play a detrimental role in the healing process of the infarcted myocardium by invasion of P.g. into the myocardium, thereby promoting apoptosis and the MMP-9 activity of the myocardium, which, in turn, can cause cardiac rupture.

In some cases, P. gingivalis induces cellular hypertrophy and MMP-9 activity via different signaling pathways in H9c2 cardiomyoblast cells. P. gingivalis medium can elevate MMP-9 activity and induce cardiomyoblast hypertrophy. P. gingivalis-induced H9c2 cell hypertrophy was mediated through p38, ERK, PI3K, calcineurin, and JNK signaling pathways, which are in a totally different regulatory pathway from P. gingivalis-elevated MMP-9 activity. P. gingivalis infection activated multiple factors via different pathways to induce the development of hypertrophy of H9c2 cardiomyoblast cells.

In some cases, P. gingivalis deteriorates Isoproterenol-Induced myocardial remodeling in mice. In some situations, stronger cardiomyocyte hypertrophy can be observed in the ISO(+)/P.g.(+) mice compared with the ISO(+)/P.g.(−) mice. The total square of randomly selected cardiomyocytes was 23% larger in the ISO(+)/P.g.(+) mice than in the ISO(+)/P.g.(−) mice. A higher level of mRNA expression in Toll-like receptor 2 and NADPH oxidase 4 in the ISO(+)/P.g.(−) mice was detected compared with the control group. A periodontal pathogen affected ISO-induced cardiac hypertrophy via oxidative stress.

In some situations, P. gingivalis-related cardiac cell apoptosis can be co-activated by p38 and extracellular signal-regulated kinase pathways. In some situations, the development of cardiac cell apoptosis can be directly induced by P. gingivalis medium. Porphyromonas gingivalis-related H9c2 cell apoptosis was mainly co-activated by p38 and ERK pathways and may be involved in death receptor-dependent (caspase 8) and mitochondria (caspase 9)-dependent apoptotic pathways. Porphyromonas gingivalis-related cardiac cell apoptosis was also partially mediated by PI3K or calcineurin signaling pathways, whereas the JNK pathway might play a protective role in P. gingivalis-related cardiac cell apoptosis.

In some situations, the miRNA-212/132 family regulates both cardiac hypertrophy and cardiomyocyte autophagy. In some situations, miR-212/132 family has a key role in cardiac hypertrophy and heart failure development. Both miR-212 and miR-132 can target and negatively regulate the expression of the Fox03 transcription factor, a powerful anti-hypertrophic and pro-autophagic factor in cardiomyocytes. The microRNA (miRNA)-212/132 family can regulate cardiac hypertrophy and autophagy in cardiomyocytes.

In some situations, Porphyromonas gingivalis-induced miR-132 regulates TNFα expression in THP-1 derived macrophages Live P. gingivalis infection induced miR-132 via TLR signaling and activation of NF-κB. Furthermore, inhibition of miR-132 expression strongly repressed the production of TNFα and increased NFE2L2 and NFAT5. Without being bound by theory, miR-132 modulates TNFα via inhibition of its target genes, which may provide a new window of opportunity to investigate therapeutic intervention for P. gingivalis-induced TNFα associated diseases such as periodontitis. Thus, formulations of the present disclosure targeting P. gingivalis can be used to address these disorders, conditions or diseases in some embodiments.

In some embodiments, the condition, disorder or disease treated by the present methods is a wound. In some embodiments, administration of a formulation of the present disclosure promotes wound closure and/or prevents or reduces P. gingivalis-induced inhibition of wound closure. In some embodiments, a novel gingipain regulatory gene in Porphyromonas gingivalis mediates host cell detachment and inhibition of wound closure. In some situations, the pgn_0361 gene is involved in regulating gingipains. The PGN_0361-defective strain of P. gingivalis exhibited reduced virulence in terms of epithelial cell detachment and inhibition of wound closure. The culture supernatant of the mutant strain can highly inhibit wound closure, which may be due to high gingipain activity.

In some situations, the capsular polysaccharide and the Arg- and Lys-gingipains of P. gingivalis influences the capacity of P. gingivalis to hinder wound healing, while LPS and the major fimbriae may have no effect. In some situations, entry of Porphyromonas gingivalis Outer Membrane Vesicles into Epithelial Cells Causes Cellular Functional Impairment. Without being bound to theory, loss of intracellular TfR due to MVs causes serious impairment of cellular migration and proliferation. Fundamental cellular operations, including DNA synthesis and ATP generation, require iron, while transferrin-TfR complexes are internalized and ferric iron is released from transferrin at endosomal pH levels. TfR degradation by P. gingivalis can cause impairment of cellular functions, and it is notable that TfR is a target molecule of the bacterium. Thus, formulations of the present disclosure targeting P. gingivalis can be used to address these disorders, conditions or diseases in some embodiments.

In some embodiments a balanced oral pathogenic bacteria and probiotics can promote wound healing via maintaining mesenchymal stem cell homeostasis. In some cases, P. gingivalis inhibits the functions of mesenchymal stem cells (MSCs) by activating NLRP3 inflammasome. LPS increase in P. gingivalis and thereby inhibits the functions of MSCs by activating NLRP3 inflammasome. Without being bound by theory, homeostasis of oral microbiomes can play a role in maintaining oral heath, provide options for the prevention and treatment of oral diseases, and have referential value for other systemic diseases caused by dysfunction of microbiota and MSCs. It is proposed that P. gingivalis lipopolysaccharide-treated human periodontal ligament stem cells (hPDLSCs) could used to study epigenetics modulations associated with periodontitis, which might be helpful to identify novel biomarkers linked to this oral inflammatory disease. Infection of hDFSCs with P. gingivalis can prolong the survival of neutrophils and increase their migration. These phenotypic changes can depend on direct cellular contacts and PPAD expression by P. gingivalis. Active JNK and ERK pathways in primed human dental follicle stem cells (hDFSCs) can be implicated in the phenotypic changes in neutrophils. In some cases, P. gingivalis can modify hDFSCs, thereby causing an immune imbalance and thus stem cell therapies may be improved and enhanced and protected by eliminating P.g. Thus, formulations of the present disclosure targeting P. gingivalis can be used to address these disorders, conditions or diseases in some embodiments.

In some embodiments, the condition, disorder or disease is age-related macular degeneration (AMD). In some situations, P. gingivalis invades human retinal pigment epithelial cells, leading to vacuolar/cytosolic localization and autophagy dysfunction. In some situations, Periodontal disease (PD) is linked to age-related macular degeneration (AMD). Porphyromonas gingivalis (Pg), a keystone oral-pathobiont, can be causative of PD, and can efficiently invades human gingival epithelial and blood-dendritic cells. Live, but not heat-killed Pg-strains can adhere to and invade ARPEs. This involves early adhesion to ARPE cell membrane, internalization and localization of Pg within single-membrane vacuoles or cytosol, with some nuclear localization apparent. In infected human cells. Pg is found in vacuoles that contain undegraded ribosomes, where Pg ferments amino acids as an energy source. Co-localized ribosomes may provide a particularly digestible source of amino acids because of their enrichment for the positively charged residues that gingipains cleave. Cytosolically free Pg quickly localizes to the rough ER to form autophagosome-like vacuoles. Our model rather suggests that Pg OMVs entering the brain through the BBB are the more likely source of this diffuse toxic insult to the brain and not a direct infection by Pg. No degradation of Pg or localization inside double-membrane autophagosomes was evident, with dividing Pg suggesting a metabolically active state during invasion. Significant downregulation of autophagy-related genes particularly, autophagosome complex, can be observed. Antibiotic protection-based recovery assay further can confirm distinct processes of adhesion, invasion and amplification of Pg within ARPE cells. P. gingivalis can invade human-RPEs, begin to characterize intracellular localization and survive within these cells. The dysbiotic periodontal pathogen P. gingivalis can efficiently invade retinal epithelial cells in high levels, replicate and are sustained within them. This invasion and autophagy evasion by the keystone species may be one of the contributing elements in the pathogenesis of retinal degenerative diseases.

In some cases, invasion of RPE by Pg and mutants can elevate AMD-related genes involved in angiogenesis; immunosuppression and complement activation which might be the target molecules for both diseases. In some situations, infection of Porphyromonas gingivalis, A Keystone Bacterium in Periodontal Microbiota, is associated with a risk for diabetic retinopathy. In some situations, there is a significant association between a specific microbe in periodontal microbiota and DR, and oral microbiota play a role in retinal eye health.

In some situations, retinal blood flow and neurovascular are coupled in patients with Alzheimer's disease and mild cognitive impairment. In patients with MCI and AD, retinal blood flow and arterial vessel diameters can be reduced compared to healthy age- and sex-matched controls. No difference was found in flicker response between groups. This indicates alterations in retinal blood flow in patients with neurodegenerative disease. Thus, formulations of the present disclosure targeting P. gingivalis can be used to address these disorders, conditions or diseases in some embodiments.

In some embodiments, the condition, disorder or disease is autism. In some situations, Autism spectrum disorder (ASD) is associated with several oropharyngeal abnormalities, including dysbiosis in the oral microbiota. Since the oral cavity is the start of the gastrointestinal tract, this strengthens and extends the notion of a microbial gut-brain axis in ASD and even raises the question whether a microbial oral-brain axis exists. It is clear that oral bacteria can find their way to the brain through a number of pathways following routine dental procedures. A connection between the oral microbiota and a number of other brain disorders has been reported.

In some situations, C1q as a regulator of brain development is implicated in autism spectrum disorders. Autism spectrum disorders (ASDs) represents a heterogeneous group of neurodevelopmental disorders with similar core features of social and communication impairments, restricted interests and repetitive behaviors. Early synaptic dysfunction due to neuroinflammatory insults may underpin the pathogenesis of abnormal brain development in some of individuals with ASDs. As a component of the innate immune response, the complement system can comprise both directly acting factors and factors that augment other components of the immune system. Beyond its involvement with innate immune responses in the brain, the complement system also plays important roles in neurodevelopment. Recent studies indicate involvement of complement component C1q in fundamental neurodevelopmental pathways and in maintenance and elimination of dendrites and synapses. The impact of aberrant complement system activity during critical windows of brain development may not only affect the local immune response but lead to atypical brain development. Thus, formulations of the present disclosure targeting P. gingivalis can be used to address these disorders, conditions or diseases in some embodiments.

In some embodiments, the condition, disorder or disease is large vessel stroke, C-IMT (Carotid Intima-media Thickness). In some cases, periodontal treatment can have an effect on carotid intima-media thickness in patients with lifestyle-related diseases. At baseline, LDL-C (low-density lipoprotein cholesterol) levels and percentage (%) of mobile teeth can be positively related to plasma IgG (immunoglobulin) antibody titer against P. gingivalis. Corresponding to improvements in periodontal clinical parameters after treatment, right and left max IMT (maximum intima-media thickness) levels cam be decreased significantly after treatment (SPT-S: start of supportive periodontal therapy, SPT-1y: at 1 year under SPT, and SPT-3y: at 3 years under SPT). P. gingivalis infection can be positively associated with progression of atherosclerosis. Without being bound by theory, routine screening using plasma IgG antibody titer against P. gingivalis and periodontal treatment under collaborative with medical and dental care may prevent cardiovascular accidents caused by atherosclerosis.

P. gingivalis infection can be associated with LDL-C level, which facilitates atherosclerosis, and that periodontal treatment, in collaboration with medical care for atherosclerosis, may contribute to improvements in max carotid IMT. Plasma P. gingivalis IgG titer may be useful for the early detection of atherosclerosis. Finally, periodontal treatment is considered to be important for preventing the onset of cerebral and myocardial infarctions caused by atherosclerosis.

In some situations, overall periodontal bacterial burden can be related to carotid IMT. In some situations, changes in clinical and microbiological periodontal profiles relate to progression of carotid intima-media thickness. In some situations, improvement in periodontal status—defined both clinically and microbiologically—is associated with less progression in carotid atherosclerosis in a randomly selected population-based sample of men and women. Accelerated atherosclerotic progression can be a mechanistic explanation linking periodontal disease and clinical CVD. Thus, formulations of the present disclosure targeting P. gingivalis can be used to address these disorders, conditions or diseases in some embodiments.

In some embodiments, the condition, disorder or disease is a systemic disease, e.g., a systemic metabolic disorder. A variety of systemic diseases can be treated by use of the present formulations, as disclosed herein. In some embodiments, the systemic disease is, without limitation, type II diabetes, insulin resistance or metabolic syndrome. Without being bound by theory, P. gingivalis virulence factors can allow the pathogen's invasion to the periodontal tissue and subsequent dissemination into the systemic circulation, increasing the risk of systemic chronic diseases such as type 2 diabetes mellitus (T2DM), cardiovascular diseases, nonalcoholic fatty liver disease (NAFLD), rheumatoid arthritis, and Alzheimer disease. As used herein, “insulin resistance” refers to the reduction or loss of the response of the target organs and tissues to the biological effects of insulin, resulting in decreased efficiency of cell uptake and utilization of glucose and the occurrence of abnormal metabolism of glucose and lipids in cells. In some cases, P. gingivalis outer membrane vesicles (OMVs) can deliver gingipains to the liver, where gingipains can regulate hepatic glycogen synthesis by attenuating insulin sensitivity through the Akt/GSK-3β signaling pathway. Thus, P. gingivalis in the oral cavity can influence hepatic glucose metabolism by decreasing insulin sensitivity in the liver cells. Further, P. gingivalis can induce insulin resistance through branched-chain amino acids (BCAA) biosynthesis. In addition, P. gingivalis/gingipain can translocate from the oral cavity to pancreatic islets and become localized primarily in B-cells, and may be epigenetically influencing development of bihormonal cells. Thus, formulations of the present disclosure targeting P. gingivalis can be used to address these disorders, conditions or diseases in some embodiments.

In some embodiments, the condition, disorder or disease is rheumatoid arthritis (RA). Without being bound by theory, antibodies against P. gingivalis have been found to be associated with RA and with anti-citrullinated protein antibodies (ACPA). Moreover, the DNA of P. gingivalis has been detected in the synovial fluid and plasma samples from patients with RA, and the coexistence of RA and periodontitis increased the probability of finding P. gingivalis DNA in these compartments. Clinical signs and symptoms of RA can improve after periodontal treatments and resolution of periodontitis. Thus, formulations of the present disclosure targeting P. gingivalis can be used to address these disorders, conditions or diseases in some embodiments.

In some embodiments, the condition, disorder or disease is cancer. In some embodiments, the cancer is, without limitation, oral, gastrointestinal, or pancreatic cancer. In some embodiments, the cancer is, without limitation, esophageal squamous cell carcinoma, head and neck (larynx, throat, lip, mouth and salivary glands) carcinoma. Without being bound to theory, P. gingivalis can promote distant metastasis and chemoresistance to anti-cancer agents and accelerate proliferation of oral tumor cells by affecting gene expression of defensins, by peptidyl-arginine deiminase and noncanonical activation of β-catenin. In some cases, the pathogen can convert ethanol to the carcinogenic intermediate acetaldehyde. In addition, P. gingivalis can be implicated in precancerous gastric and colon lesions, esophageal squamous cell carcinoma, head and neck (larynx, throat, lip, mouth and salivary glands) carcinoma, and pancreatic cancer. P. gingivalis can have systemic tumorigenic effects in addition to the local effects in its native territory, the oral cavity. Thus, formulations of the present disclosure targeting P. gingivalis can be used to address these disorders, conditions or diseases in some embodiments.

In some embodiments, a formulation of the present disclosure may be administered in conjunction with one or more cancer therapy agents, e.g., chemotherapeutic agent, to enhance the therapeutic effect of the cancer therapy agent. In some embodiments, the cancer therapy agent is a small molecule drug, or an immunotherapeutic agent. In some cases P. gingivalis, its OMVs and/or gingipains have been found to cause an overall immunosuppression of the host, suppressing the adaptive immune system and altering the innate immune system. Adjuvant therapy of eliminating P.g. for improved outcomes for current and future chemotherapies. In some cases, P. gingivalis can inhibit drug induced apoptosis as well as necrosis (at least the LDH release) in the esophageal squamous cell carcinoma cell line EC0706. When the cancer cells are infected with P. gingivalis prior to the treatment with cisplatin, both apoptosis and necrosis is significantly reduced. Tumor xenografts composed of P. gingivalis-infected OSCC cells can exhibit a higher resistance to Taxol through Notch1 activation, as compared with uninfected cells. Furthermore, P. gingivalis-infected OSCC cells can form more metastatic foci in the lung than uninfected cells. Sustained infection with P. gingivalis, can promote distant metastasis of oral cancer, as well as its resistance to anti-cancer agents. Oral cancer cells sustainedly infected with Porphyromonas gingivalis can exhibit resistance to Taxol and have higher metastatic potential. Thus, in some embodiments, treating and eliminating P.g. with the formulations improves multiple primary, secondary and adjuvant related cancer treatments.

In some embodiments, the condition, disorder or disease to be treated by the present methods is a lung disease, such as non-smokers lung cancer and aspiration pneumonia. In some embodiments, targeting inflammation with anti-inflammatory therapy can lead to a significantly lower rate of recurrent cardiovascular events independent of lipid-level lowering. There can be a substantial lowering of non-smokers lung cancer with anti-inflammatory therapy targeting the interleukin-1b innate immunity pathway leading to significantly lower cancer mortality consistent with experimental data relating to interleukin-1b.

In some situations, Porphyromonas gingivalis is the primary microbial pathogen as single source driver of inflammation and it's multiple NLRP3/IL-1 β pathway mediated diseases including Atherosclerosis and Cardiovascular disease. In some situations, Infection with P. gingivalis can trigger the activation of NLRP3 and AIM2 inflammasomes via TLR2 and TLR4 signaling, leading to IL-1β secretion and pyroptic cell death. In addition, P. gingivalis-induced NLRP3 inflammasome activation can be dependent on ATP release, K+ efflux, and cathepsin B. In some embodiments, any of the formulation can be used to alter TLR4 signaling.

Without being bound by theory, the periodontopathogen Porphyromonas gingivalis has been shown to have several mechanisms of modulating innate immunity by limiting the activation of the NLRP3 inflammasome. The innate immune system can be the first line of defense against microbial pathogens. P. gingivalis can modify innate immunity by affecting inflammasome activity.

Wild type challenge of apolipoprotein E-deficient, spontaneously hyperlipidemic (ApoE) mice with P. gingivalis can increase IL-1β, IL-18, and TNF-α production in peritoneal macrophages and gingival or aortic gene expression of the NOD-like receptor family, NLRP3, IL-1β, pro-IL-1β and pro-caspase-1.

In some situations, outer membrane vesicles derived from Porphyromonas gingivalis can induce cell death with disruption of tight junctions in human lung epithelial cells. P. gingivalis OMVs can cause cell damage with cell membrane destruction in Human lung epithelial cell. P. gingivalis OMVs suppressed cell viability of Human lung epithelial cell by causing apoptosis. P. gingivalis OMVs translocated through oral cavity may be a trigger for inflammation of airway diseases. Thus, formulations to this target can be used to address this in some embodiments.

In some situations, P. gingivalis OMVs can induce cell death by destroying the barrier system in lung epithelial cells. P. gingivalis OMVs may be a factor in the engagement of periodontitis with respiratory system diseases.

In some situations, Porphyromonas gingivalis is an aggravating factor for chronic obstructive pulmonary disease patients with periodontitis. The microbial analysis of sputum from COPD patients with CP to detect periodontal pathogen Porphyromonas gingivalis (P. gingivalis) both before and after nonsurgical periodontal therapy. A decrease in the count of P. gingivalis and decreased periodontal indices values can be observed in COPD patients with periodontitis after nonsurgical periodontal therapy. Lung function test (forced expiratory volume in the first/forced vital capacity) can be improved in COPD patients with periodontitis after nonsurgical periodontal therapy. In some embodiments, nonsurgical periodontal therapy can be a part of treatment protocol in COPD patients because it helps in reducing the P. gingivalis count and improves the lung function.

In some situations, gingipains are factors in the development of aspiration pneumonia caused by Porphyromonas gingivalis. Aspiration pneumonia can be a life-threatening infectious disease often caused by oral anaerobic and periodontal pathogens such as Porphyromonas gingivalis. This organism can produce proteolytic enzymes, known as gingipains, which can manipulate innate immune responses and promote chronic inflammation. P. gingivalis W83 gingipains can have a role in bronchopneumonia, lung abscess formation, and inflammatory responses. Gingipains can be important for clinical symptoms and infection-related mortality. Pathologies caused by wild-type (WT) P. gingivalis W83, including hemorrhage, necrosis, and neutrophil infiltration, can be absent from lungs infected with gingipain-null isogenic strains or WT bacteria preincubated with gingipain-specific inhibitors. Damage to lung tissue can be correlated with systemic inflammatory responses, as manifested by elevated levels of TNF, IL-6, IL-17, and C-reactive protein. These effects can be dependent on gingipain activity. Gingipain activity can also be implicated in the observed increase in IL-17 in lung tissues. Furthermore, gingipains can increase platelet counts in the blood and activated platelets in the lungs. Arginine-specific gingipains can make a greater contribution to P. gingivalis-related morbidity and mortality than lysine-specific gingipains. Thus, inhibition of gingipain may be a useful adjunct treatment for P. gingivalis-mediated aspiration pneumonia.

One of the pathogenic outcomes of P. gingivalis-triggered aspiration pneumonia can be thrombocytosis. Thrombocytosis can be associated with inflammatory disease, and the platelet count can be an acute-phase response to inflammation induced by P. gingivalis.

Animals challenged with WT P. gingivalis can show a sharp increase in TNF-α, IL-6, and MCP1 levels. The lungs from infected animals can show clear increases in MPO levels, which are indicative of neutrophil infiltration. The highest MPO concentrations can be detected in lung homogenates from animals infected with WT P. gingivalis, whereas those from mice infected with the ΔKgp and ΔRgp strains can show significantly lower MPO activity.

Intratracheal inoculation with either WT P. gingivalis or ΔKgp can lead to a significant increase in IL-17 expression in lung tissue and peripheral blood. Proteolytically active gingipains can modulate the course of P. gingivalis-associated aspiration pneumonia and aggravate the host immune response. P. gingivalis-derived enzymes can play an important role not only during chronic disease (e.g. periodontitis) but also during acute, life-threatening pneumonia. In some situations, TLR2 is implicated in Early Innate Immune Response to Acute Pulmonary Infection with Porphyromonas gingivalis in Mice. The periodontal pathogen Porphyromonas gingivalis is implicated in certain systemic diseases including atherosclerosis and aspiration pneumonia. This organism can induce innate responses predominantly through TLR2, which also mediates its ability to induce experimental periodontitis and accelerate atherosclerosis. TLR2-deficient mice can elicit reduced proinflammatory or antimicrobial responses (KC, MIP-1, TNF-, IL-6, IL-12p70, and NO) in the lung and exhibited impaired clearance of P. gingivalis compared with normal controls. However, the influx of polymorphonuclear leukocytes into the lung and the numbers of resident alveolar macrophages (AM) can be comparable between the two groups. TLR2 signaling can be important for in vitro killing of P. gingivalis by polymorphonuclear leukocytes or AM and, moreover, the AM bactericidal activity can require NO production. Strikingly, AM can be more potent than peritoneal or splenic macrophages in P. gingivalis killing, attributed to diminished AM expression of complement receptor-3 (CR3), which is exploited by P. gingivalis to promote its survival. Without being bound by theory, the selective expression of CR3 by tissue macrophages and the requirement of TLR2 inside-out signaling for CR3 exploitation by P. gingivalis indicates that the role of TLR2 in host protection may be contextual. In some embodiments, TLR2 may mediate destructive effects, as seen in models of experimental periodontitis and atherosclerosis, and the same receptor can confer protection against P. gingivalis in acute lung infection.

In some situations, periodontopathic anaerobes are involved in aspiration pneumonia. Porphyromonas gingivalis and Treponema denticola can coexist in chronic periodontitis lesions. In some situations, a mixed culture of P. gingivalis and T. denticola can be inoculated into the mouse trachea; and cause an infection inducing inflammatory cytokine production and pneumonia. In another series of investigations, professional oral health care (POHC), mainly cleansing administered by dental hygienists once a week for 24 months to elderly persons requiring daily care, can result in the reduction of the number of total anaerobes, Candida albicans, and Staphylococcus species and in the number of cases of fatal aspiration pneumonia. The POHC treatment of elderly persons for 6 months in the winter season can reduce the salivary levels of protease, trypsin-like activity, and neuraminidase and also can decrease the frequency of influenza cases.

In some embodiments, Porphyromonas gingivalis can induce inflammatory responses and promote apoptosis in lung epithelial cells infected with H1N1 via the Bcl-2/Bax/Caspase-3 signaling pathway P. gingivalis may induce the production of a large number of inflammatory cytokines in lung epithelial cells. Lung epithelial cells infected with H1N1 and P. gingivalis can lead to the promoted production of inflammatory cytokines and the expression of iNOS, which may have also increased the accumulation of NO, resulting in an increased proportion of lung epithelial cells undergoing apoptosis via the Bcl-2/Bax/caspase-3 signaling pathway. Following BEAS-2B cell infection with P. gingivalis and H1N1, the concentrations of TNF-α, IL-1β and IL-6 in the supernatant can be significantly increased at each time point, compared with the H1N1 and P. gingivalis alone groups. These results demonstrated that lung epithelial cells infected with H1N1 and P. gingivalis can promote the production of inflammatory cytokines.

In some situations, Porphyromonas gingivalis modulates Pseudomonas aeruginosa-induced apoptosis of respiratory epithelial cells through the STAT3 signaling pathway. P. gingivalis invasion can transiently inhibit P. aeruginosa-induced apoptosis in respiratory epithelial cells via the signal transducer and activator of transcription 3 (STAT3) signaling pathway. The activated STAT3 can up-regulate the downstream anti-apoptotic moleculars survivin and B-cell leukemia-2 (bcl-2). This process can be accompanied by down-regulation of pro-apoptosis molecular Bcl-2-associated death promoter (bad) and caspase-3 activity inhibition. In addition, the activation of the STAT3 pathway can be affected by P. gingivalis in a dose-dependent manner. Finally, co-invasion of P. aeruginosa and P. gingivalis can lead to greater cell death compared with P. aeruginosa challenge alone. These results indicate that regulation of P. aeruginosa-induced apoptosis by P. gingivalis can contribute to the pathogenesis of respiratory disease. Thus, formulations of the present disclosure targeting P. gingivalis can be used to address these disorders, conditions or diseases in some embodiments.

In some embodiments, oral cancer cells sustainedly infected with Porphyromonas gingivalis can exhibit resistance to Taxol and can have higher metastatic potential. Sustained infection with P. gingivalis, a major pathogen responsible for chronic periodontitis, can promote distant metastasis of oral cancer, as well as its resistance to anti-cancer agents. Thus, formulations of the present disclosure targeting P. gingivalis can be used to address these disorders, conditions or diseases in some embodiments.

In some embodiments, the condition, disorder or disease treated by the present methods is Glioma. Without being bound by theory, Cathepsin B plays a critical role in inducing Alzheimer's Disease-like phenotypes following chronic systemic exposure to lipopolysaccharide from Porphyromonas gingivalis in mice. In some cases, systemic exposure to LPS from Porphyromonas gingivalis can induce AD-like phenotypes; Cathepsin B is implicated in inducing microglia-mediated neuroinflammation; Cathepsin B is implicated in inducing microglia-dependent Aβ accumulation in neurons. In some situations, a strong association can exist between periodontitis and accelerated cognitive decline in Alzheimer's disease (AD). Cathepsin (Cat) B can play a critical role in the initiation of neuroinflammation and neural dysfunction following chronic systemic exposure to lipopolysaccharide from Porphyromonas gingivalis (PgLPS). Thus, formulations of the present disclosure targeting P. gingivalis can be used to address these disorders, conditions or diseases in some embodiments.

In some embodiments, the condition, disorder or disease is a gut microbiome-related disorder. A variety of gut microbiome-related disorder can be treated by the formulations of the present disclosure. In some embodiments, the gut microbiome-related disorder is an intestinal disorder such as, without limitation, inflammatory bowel disease, irritable bowel syndrome (IBS), coeliac disease. In some embodiments, the gut microbiome-related disorder is an extra-intestinal disorder such as, without limitation, allergy, asthma, metabolic syndrome, cardiovascular disease, and obesity. Without being limited by theory, endotoxemia that may cause metabolic disorders can be related to changes in the gut microbiota caused by oral bacteria, e.g., P. gingivalis. In some cases, periodontal inflammation can affect the mechanical and immune barrier functions of the gut. Orally administered P. gingivalis can cause composition shifts in the gut microbiota and increase serum endotoxin and inflammatory markers, and affect the gut immune system. In addition, P. gingivalis has been associated with NAFLD and non-alcoholic steatohepatitis (NASH). P. gingivalis can be detected in the gut of the NAFLD and NASH patients. Thus, formulations of the present disclosure targeting P. gingivalis can be used to address these disorders, conditions or diseases in some embodiments.

In some embodiments, the condition, disorder or disease is a cognitive disorder. In some embodiments, the condition, disorder or disease is dementia associated with microvasculature defects. In some embodiments, the condition, disorder or disease is microvascular defects Parkinson's.

In some situations, cerebral oxidative stress and microvasculature defects are implicated in TNF-α Expressing Transgenic and Porphyromonas gingivalis-Infected ApoE−/− Mice. There can be a major difference in the hippocampi of P. gingivalis-infected and sham-infected ApoE−/− mice, in terms of increased protein carbonyl/oxidized protein content in the hippocampal micro-vasculature. Hippocampal microvascular structures and the homeostasis of the brain can be at risk from elevated oxidative stress and oxidative protein damage, following P. gingivalis infection. Without being bound by theory, following recurrent episodes of active periodontal disease, there exists a possibility for the development of a defective BBB, post neuroinflammation-mediated cerebral parenchymal tissue injury. The rising levels of intrinsic and extrinsic sources of cytokines, oxidative stress, and developing BBB defects may be implicated as early modifiers of neurodegenerative and disease severity leading to deteriorating memory. Infection with P. gingivalis can be interpreted as one of the plausible mechanisms by which a susceptible host can develop dementia.

A variety of cognitive disorders can be treated by the formulations of the present disclosure. In some embodiments, the cognitive disorder is Alzheimer's disease (AD). Without being bound by theory, periodontitis has been shown to be a risk factor for AD and a more rapid cognitive decline. In some cases, genetic predisposition, P. gingivalis infection and microglia could promote neurodegeneration typical of that reported for AD. P. gingivalis specific cell free DNA can be detected in the cerebrospinal fluid of AD patients and the pathogen's protease virulence factors, arginine-gingipain (Rgp) and lysine-gingipain (Kgp), can be found in the brains of over 90% of AD patients and can correlate with tau and ubiquitin pathology. Concurrently, there is evidence of Pg OMVs either targeting and/or seeking out tissues higher in arginine and lysine amino acids P. gingivalis can invade and persist in mature neurons, which, once infected, can display signs of AD-like neuropathology, including the accumulation of autophagic vacuoles and multivesicular bodies, cytoskeleton disruption, an increase in phosphotau/tau ratio, and synapse loss. Gingipains of P. gingivalis can digest tau protein into peptide fragments, some of which include tau residues prone to phosphorylation and some of which include two of the four microtubule binding domains that form paired/straight helical filaments constituting neurofibrillary tangles (NFTs). In some cases, Gingipains have been found to be neurotoxic in vivo and in vitro, having detrimental effects on tau. P. gingivalis lipopolysaccharide (LPS) can activate the phosphoinositide 3-kinase/Akt (PI3K/AKT) pathway and increase expression of glycogen synthase kinases-3 beta (GSK-3B), which can phosphorylate tau. P. gingivalis can invade and survive in neurons and generate intra-neuronal gingipains that are proteolytically active, leading to neurodegeneration associated with AD. This observation is consistent with studies looking at the neuro-anatomical analysis of Pg associated genes (gingipains) which mark cholinergic neurons, basal forebrain and anterior hypothalamic regions; regions near ventricles and peripheral neurons are also enriched, suggesting relevance to Pg brain entry. In addition to amyloid plaques and neurofibrillary tangles, functional studies suggest that hypothalamic dysfunction is a common event in AD, often early in the course of disease. Although there are evidences indicating that certain hypothalamic regions are also affected in Frontal temporal lobe dementia (FTD), specifically those that correlate with abnormal eating behaviors, they are different to those affected in AD. A possible explanation could be that the hypothalamic region, which controls body innate immunity, is affected in the earliest pro-domal stages of AD, but not in FTD. The apparently AD-specific salivary Lf reduction may thus not only be useful in the differential diagnosis but could also provide important insights into selective immune vulnerability in neurodegenerative diseases. As mentioned above the secretion of salivary proteins is controlled by cholinergic parasympathetic nerves that release acetylcholine, evoking the secretion of saliva by acinar cells in the salivary gland. These parasympathetic nerves are connected with the hypothalamus. We propose that early hypothalamic Aβ accumulation is associated with Pg OMVs gingipains deposition found in postmortem brain tissue with the upregulation of ER translocation genes in the context of Alzheimer's disease. This could be an early switch that begins the loss of control and disrupt hypothalamic function affecting salivary gland regulation that ultimately results in reduced salivary Lf secretion. Pg is known to degrade Lf for its major early iron source in oral cavity. Should neural based impairment of the salivary glands produce a decline in the steady-state level of Lf, a major switch in an otherwise delicate balance between Pg and the oral cavity may ensue. More specifically, the diminishing oral salivary iron source would further signal to Pg the need for new iron source. In some embodiments, a subject with Down's syndrome is at increased risk of developing AD.

In some cases, P. gingivalis can induce migration of microglial cells to sites of infection in the brain, through activation of mitogen-activated protein kinase/extracellular signal-regulated kinase (ERK) kinase/ERK pathway. P. gingivalis can induce synthesis of matrix metalloproteinases (MMPs), which can have an important role in neuroinflammatory disorders including AD. Oral infection with P. gingivalis can result in the pathogen spreading to the brain and activating microglia. P. gingivalis can downregulate TREM-2 expression in microglia. Lack of TREM-2 protein may accelerate aging processes, neuronal cell loss and reduce microglial activity leading to neuroinflammation. P. gingivalis can contribute to development of AD inflammatory pathology through mechanisms involving acute phase proteins, cytokines and the complement cascade where neurons would be attacked. Inappropriate complement activity can play a significant role in AD pathophysiology.

LPS, a virulence factor of P. gingivalis, in the brain can initiate neuroinflammation in the form of microglial cell activation, and the neuroinflammatory response can be stronger with age. Age-associated priming of microglia may have a role in exaggerated inflammation induced by activation of the peripheral immune system. In some cases, P. gingivalis can cause an imbalance in M1/M2 activation in macrophages, resulting in a hyperinflammatory environment that promotes the pathogenesis of periodontitis, and leptomeningeal cells can transduce inflammatory signals from peripheral macrophages to brain resident microglia exposed to P. gingivalis LPS. In microglia, P. gingivalis LPS can increase the production of cathepsin B and pro-forms of caspase-1 and IL-1β through activation of Toll-Like Receptor (TLR) 2/NF-kB signaling. Cathepsin B is implicated in in P. gingivalis LPS-induced AD-like pathology, and may be necessary for the induction of AD-like pathology following chronic systemic exposure to P. gingivalis LPS. In some cases, treating periodontitis can lead to improvements in cognition. A chronic infection of the brain with P. gingivalis can cause serious consequences for the BBB and subsequent mental health. Thus, formulations of the present disclosure targeting P. gingivalis can be used to address these disorders, conditions or diseases in some embodiments.

In some embodiments, the condition, disorder or disease is an age-related disorder. Without being bound by theory, P. gingivalis can impact cellular biochemical pathways that are associated with improved longevity or shortened life spans, e.g., by regulating autophagy and apoptosis, modulating the mTORC1 pathway, or targeting cellular senescence by selectively eliminating senescent cells. Disrupted autophagy has been linked to numerous diseases including Parkinson's disease, and type 2 diabetes. In some cases, P. gingivalis minor (Mfa1) fimbriae can manipulate dendritic cell (DC) signaling to perturb both autophagy and apoptosis. Mfa1 can induce Akt nuclear localization and activation, and ultimately can induce mTOR in DCs. P. gingivalis can promote DC survival by increasing anti-apoptotic Bcl2 protein expression and decreasing pro-apoptotic proteins Bim, Bax and cleaved caspase-3. In some cases, lipophilic outer membrane vesicles (OMV) shed from P. gingivalis can promote monocyte unresponsiveness to live P. gingivalis. Full reactivity to P. gingivalis can be restored by inhibition of mTOR signaling, which can promote Toll-like receptor 2 and Toll-like receptor 4 (TLR2/4)-mediated tolerance in monocytes. Without being bound by theory, it is thought that P. gingivalis, a facultative intracellular microbe, may damage not only cell membranes but also the mitochondrion, triggering a bioenergetic crisis and NLRP3-induced cellular senescence. Moreover, age-related brain LPS elevation may trigger intracellular iron migration, an innate immune response to withhold iron from pathogens.

Without being bound by theory, the major surface glycoproteins of P. gingivalis—Pgm6 and Pgm7, also called outer membrane protein A-like proteins (OmpALPs)—mediate resistance to the bactericidal activity of human serum, and specifically protect P. gingivalis from the bactericidal activity of LL-37 and from innate immune recognition by TLR4. LL-37 proteolysis by P. gingivalis may provide neighboring dental plaque species with resistance to LL-37, which in turn can benefit P. gingivalis. Thus, formulations of the present disclosure targeting P. gingivalis can be used to address these disorders, conditions or diseases in some embodiments.

In some embodiments, the condition, disorder or disease is an aneurysm, e.g., cerebral or abdominal aneurysm. In some cases, pro-inflammatory response elicited by Porphyromonas Gingivalis lipopolysaccharide exacerbates the rupture of experimental cerebral aneurysms. Porphyromonas gingivalis LPS can exacerbate vascular inflammation and can enhance the rupture of intracranial aneurysms.

In some situations, CPI can be significantly higher in patients with IAs than the controls (2.7 vs 1.9, p<0.05) and their DNA level of subgingival plaques and their plasma IgG titers of Pg can also be higher. Periodontal disease can be more severe and the plasma IgG titers of Pg can be higher in patients with ruptured—than unruptured IAs, suggesting that Pg is associated not only with the formation but also the rupture of IAs. Severe periodontal disease and Pg infection may be involved in the pathophysiology of IAs.

In some situations, the condition, disorder or disease is depression. Without being bound by theory, it is thought Porphyromonas gingivalis can induce depression via downregulating p75NTR-mediated BDNF maturation in astrocytes. In some embodiments, Pg-LPS decreases the level of astrocytic p75NTR and then downregulates BDNF maturation, leading to depression-like behavior in mice. Pg can be a modifiable risk factor for depression. In some embodiments, Porphyromonas gingivalis (Pg) can induce depression-like behaviors; Astrocytic p75NTR can be decreased in Pg-colonized mice; Overexpression of p75NTR in astrocytes can rescue depressive behaviors; Antibiotic therapy can ameliorate Pg-induced depressive behavior in mice. Thus, formulations of the present disclosure targeting P. gingivalis can be used to address these disorders, conditions or diseases in some embodiments.

In some embodiments, the condition, disorder or disease is peri-implantitis. In some situations, oral infection with Porphyromonas gingivalis can induce peri-implantitis, and can be implicated in bone loss and the local inflammatory response. Porphyromonas gingivalis infection can induce greater bone loss around implants than around teeth. In non-infected animals, the presence of the implant can correlate with elevated expression of Il-10, Foxp3 and Rank1/Opg ratio, while Tnf-α levels can be decreased relative to tissue around teeth. Six weeks following infection, Tnf-α can be increased significantly while the expression of Foxp3 can be decreased in the tissue around the implants. Oral infection with P. gingivalis of mice with implants can induce bone loss and a shift in gingival cytokine expression. In some situations, the fimA type Ib genotype of P. gingivalis can play a role in the destruction of peri-implant tissue, indicating that it may be a distinct risk factor for peri-implantitis.

In some situations, cytokine and matrix metalloproteinase expression in fibroblasts from peri-implantitis lesions can be observed response to viable Porphyromonas gingivalis. Fibroblasts from peri-implantitis and periodontitis lesions can exhibit a more pronounced inflammatory response to the P. gingivalis challenge than fibroblasts from healthy donors. Without being bound by theory, they may therefore be involved in the development of inflammation in peri-implantitis and periodontitis. Moreover, the sustained upregulation of inflammatory mediators and MMP-1 in peri-implantitis fibroblasts may play a role in the pathogenesis of peri-implantitis.

In some embodiments, the condition, disorder or disease is bone loss or osteoporosis. In some cases periodontal disease and associated bone loss by Porphyromonas gingivalis Stimulates bone resorption by enhancing RANKL (Receptor Activator of NF-κB Ligand) through Activation of Toll-like Receptor 2 in Osteoblasts. LPS P. gingivalis and Pam2 can enhance osteoclast formation in periosteal/endosteal cell cultures by increasing RANKL. LPS P. gingivalis and Pam2 can also up-regulate RANKL and osteoclastic genes in vivo, resulting in an increased number of periosteal osteoclasts and immense bone loss in wild type mice but not in Tlr2-deficient mice. In some cases, LPS P. gingivalis can stimulate periosteal osteoclast formation and bone resorption by stimulating RANKL in osteoblasts via TLR2. Without being bound by theory, this effect might be important for periodontal bone loss and for the enhanced bone loss seen in rheumatoid arthritis patients with concomitant periodontal disease. In some situations, activation of TLR2 in osteoblasts by P. gingivalis increases RANKL production, osteoclast formation, and bone loss both ex vivo and in vivo. P. gingivalis can stimulate alveolar bone loss can cause a more severe loss of juxta-articular bone in RA. In some situations, TLR2, which is highly expressed in RA synovium, is not only activated by pathogen-associated molecular patterns such as P. gingivalis but also by endogenous ligands present in RA synovium such as gp96 and Snapin. There may be a role of endogenous ligands in the pathogenesis of RA bone erosions. Moreover, genetic or antibody-mediated inactivation of TLR2 can reduce cytokine production in P. gingivalis-stimulated neutrophils or macrophages, suggesting that TLR2 plays a non-redundant role in the host response to P. gingivalis. In the absence of MyD88, inflammatory TLR2 signaling in P. gingivalis-stimulated neutrophils or macrophages can depend upon PI3K. TLR2-PI3K signaling may be implicated in P. gingivalis evasion of killing by macrophages, since their ability to phagocytose this pathogen can be reduced in a TLR2 and PI3K-dependent manner. Moreover, within those cells that did phagocytose bacteria, TLR2-PI3K signaling can block phago-lysosomal maturation, thereby revealing a novel mechanism whereby P. gingivalis can enhance its intracellular survival. In some cases, P. gingivalis can uncouple inflammation from bactericidal activity by substituting TLR2-PI3K in place of TLR2-MyD88 signaling. P. gingivalis can be a keystone pathogen, which can manipulate the host inflammatory response in a way that promotes bone loss but not bacterial clearance. Without being bound by theory, modulation of these host response factors may be a therapeutic approach to improve outcomes in disease conditions associated with P. gingivalis.

In some cases, periodontal pathogenic bacteria as well as intestinal dysbiosis are involved in the determinism of bone mineral density BMD loss, and contribute to the onset and worsening of osteoporosis OP. Thus, formulations of the present disclosure targeting P. gingivalis can be used to address these disorders, conditions or diseases in some embodiments.

In some situations, early host-microbe interaction is implicated in a peri-implant oral mucosa-biofilm model. In some situations, various factors (V. dispar, P. gingivalis, immune cells) could be involved in the disruption or maintenance of homeostasis. Thus, formulations of the present disclosure targeting P. gingivalis can be used to address these disorders, conditions or diseases in some embodiments.

In some embodiments methods of the recent disclosure include administering to the subject the formulation of the present disclosure in conjunction with one or more treatments of telomer length and/or prevention with various drugs and or natural supplements. Without being bound by theory, it has been shown that shorter telomere lengths are associated with a diagnosis of periodontitis and their measures correlate with the oxidative stress and severity of disease. Thus, formulations with any one of the formulations of the present disclosure targeting P. gingivalis can be used to address these disorders, conditions or diseases in some embodiments.

Also provided herein are methods of preventing one or more conditions, disorders, or diseases, as disclosed herein, by administering to a subject, e.g., a subject at risk of developing the condition, disorder, or disease, an effective amount of a formulation of the present disclosure, to thereby prevent the condition, disorder, or disease or developing. In some embodiments, the subject is predisposed to developing the condition, disorder, or disease. In some embodiments, the subject has a past history of an P. gingivalis infection and/or condition or disease associated with a P. gingivalis infection, as disclosed herein. In some embodiments, the subject is genetically predisposed to develop the condition, disorder, or disease. In some embodiments, the method includes identifying a subject predisposed to developing any one or more of the conditions, disorders, or diseases, as disclosed herein, and administering to the subject an effective amount of a formulation of the present disclosure to thereby prevent, reduce the likelihood and/or delay the onset of the conditions, disorders, or diseases.

In any of the above methods, the formulation can be administered in conjunction with one or more additional therapeutic agents for treating or preventing the condition, disease or disorder. In some embodiments, a therapeutic agent for treating or preventing the condition, disease or disorder, as disclosed herein, can be administered to a subject in need thereof in at a therapeutically effective amount, and an effective amount of the formulation of the present disclosure can be administered to the subject. Administration of the formulation can in some embodiments improve or enhance the therapeutic effect of the other therapeutic agent. As used herein, a first agent administered in conjunction with administering a second agent can include administering the first agent before, after, or simultaneously as the second agent. In some embodiments, the first agent and second agent are administered within an interval such that the therapeutic effect of the first agent is present in the subject when the second agent is administered to the subject.

By way of non-limiting examples, the formulation can in some embodiments be administered in conjunction with one or more additional therapeutic agents for treating or preventing a vascular disease, as disclosed herein. In some embodiments, the other therapeutic agent includes a serum lipid lowering agent. Any suitable serum lipid lowering agent can be used. In some embodiments, the serum lipid lowering agent includes, without limitation, statins (e.g., atorvastatin, cerivastatin, fluvastatin, lovastatin, mevastatin, pitavastatin, pravastatin, rosuvastatin, simvastatin), Nicotinic acid (Niacin) (e.g., NIACOR, NIASPAN (slow release niacin), SLO-NIACIN (slow release niacin), CORDAPTIVE (laropiprant)), Fibric acid (e.g., LOPID (Gemfibrozil), TRICOR (fenofibrate), Bile acid sequestrants (e.g., QUESTRAN (cholestyramine), colesevelam (WELCHOL), colestipol (COLESTID)), Cholesterol absorption inhibitors (e.g., ZETIA (ezetimibe)), PPAR gamma agonsits, PPAR alpha/gamma agonists, squalene synthase inhibitors, CETP inhibitors, anti-hypertensives, anti-diabetic agents (such as sulphonyl ureas, insulin, GLP-1 analogs, DDPIV inhibitors, e.g., metaformin), ApoB modulators, such as mipomersan, MTP inhibitoris and/or arteriosclerosis obliterans treatments.

The formulation can in some embodiments be administered in conjunction with one or more additional therapeutic agents for treating or preventing cancer, as disclosed herein. In some embodiments, the other therapeutic agent includes an anti-cancer therapeutic that is a small molecule drug or immunotherapeutic agent. Any suitable small molecule drug or immunotherapeutic agent can be used.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein, table numbering is assigned so as to provide a shorthand reference to the example, if any, that the table is discussed. Tables that are only discussed in the detailed description can be denoted by a sub I value (e.g., 0.1). This is not meant to limit the relevance or discussion or implications of the tables, but to serve as a quick reference guide.

In some embodiments, the technology described herein is further illustrated by the following arrangements which in no way should be construed as being further limiting.

1. An antibody formulation comprising an antibody, wherein the antibody comprises:
a CDR1 that is the CDR1 of SEQ ID NO:1;
a CDR2 that is the CDR2 of SEQ ID NO: 1;
a CDR3 that is the CDR3 of SEQ ID NO: 1;
a CDR4 that is the CDR4 of SEQ ID NO: 2;
a CDR5 that is the CDR5 of SEQ ID NO: 2; and
a CDR6 that is the CDR6 of SEQ ID NO: 2;
wherein the formulation is adjusted to a final pH of about 6.0;
wherein the formulation consists essentially of: 0.1 mg/mL antibody, 20 mM histidine, 0.01% Poloxamer P188, and sucrose at a concentration of about 85 mM to about 200 mM.

2. An antibody formulation comprising an antibody, wherein the antibody comprises:
a VH segment that has at least 75%, 79%, 80%, 90% or 100% identity to SEQ ID NO: 1; and
a VL segment that has at least 75%, 79%, 80%, 90% or 100% identity to SEQ ID NO: 2;
wherein the formulation is adjusted to a final pH of about 6.0;
wherein the formulation consists essentially of: 0.1 mg/mL antibody, 20 mM histidine, 0.01% Poloxamer P188, and sucrose at a concentration of about 85 mM to about 200 mM.

3. An antibody formulation, wherein the formulation consists essentially of: an antibody, a histidine, a detergent, and a sugar;
wherein the formulation is adjusted to a final pH of about 6.0;
wherein the antibody comprises:
a CDR1 that is the CDR1 of SEQ ID NO:1;
a CDR2 that is the CDR2 of SEQ ID NO: 1;
a CDR3 that is the CDR3 of SEQ ID NO: 1;
a CDR4 that is the CDR4 of SEQ ID NO: 2;
a CDR5 that is the CDR5 of SEQ ID NO: 2; and
a CDR6 that is the CDR6 of SEQ ID NO: 2.

4. An antibody formulation, wherein the formulation consists essentially of: an antibody, a histidine, a detergent, and a sugar;
wherein the formulation is adjusted to a final pH of about 6.0;
wherein the antibody comprises:
a VH segment that has at least 75%, 79%, or 80% identity to SEQ ID NO:1;
and
a VL segment that has at least 75%, 79%, or 80% identity to SEQ ID NO: 2.

5. The antibody formulation of any one of arrangements 3 or 4, wherein the detergent is Poloxamer P188.

6. The antibody formulation of any one of arrangements 3-5, wherein the sugar is sucrose.

7. The antibody formulation of any one of arrangements 3-6, wherein the formulation consists essentially of: 0.1 mg/ml antibody, 20 mM histidine, 0.01% Poloxamer P188, and sucrose at a concentration of about 85 mM to about 200 mM.

8. An antibody formulation comprising histidine and an antibody, wherein the antibody targets P. gingivalis.

9. The antibody formulation of arrangement 8, wherein the antibody comprises at least one, two, three, four, five, or all six of the CDR regions of SEQ ID NOS: 1-2.

10. The antibody formulation of any one of claim 8 or 9, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

11. An antibody formulation comprising histidine and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

12. The antibody formulation of arrangement 11, wherein the antibody targets P. gingivalis.

13. The antibody formulation of any one of arrangements 11 or 12, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

14. The antibody formulation of any one of arrangements 11-13, wherein the antibody comprises SEQ ID NO: 1 and SEQ ID NO: 2.

15. The antibody formulation of any one of arrangements 8-14, wherein the formulation further comprises a sugar and/or a detergent.

16. The antibody formulation of arrangement 15, wherein the sugar is sucrose.

17. The antibody formulation of any one of arrangements 15 or 16, wherein the detergent is Poloxamer P188.

18. The antibody formulation of any one of arrangements 8-17, wherein the antibody is present at a concentration of about 0.9-0.12 mg/mL.

19. The antibody formulation of any one of arrangements 8-18, wherein the antibody is present at a concentration of 0.1 mg/mL.

20. The antibody formulation of any one of arrangements 8-19, wherein the histidine is present at a concentration of about 1 to about 40 mM.

21. The antibody formulation of any one of arrangements 8-20, wherein the histidine is present at a concentration of 20 mM.

22. The antibody formulation of any one of arrangements 8-21, wherein the formulation has a pH of about 4 to about 8.

23. The antibody formulation of any one of arrangements 8-22, wherein the formulation has a pH of 6.

24. The antibody formulation of any one of arrangements 8-22, wherein the formulation has a pH of 5.8.

25. The antibody formulation of any one of arrangements 8-24, wherein the formulation further comprises Poloxamer P188 at a concentration of about 0.001-0.1% weight/volume.

26. The antibody formulation of any one of arrangements 8-25, wherein the formulation further comprises Poloxamer P188 at a concentration of about 0.01% weight/volume.

27. The antibody formulation of any one of arrangements 8-26, wherein the formulation further comprises sucrose at a concentration of about 85 to 200 mM.

28. The antibody formulation of any one of arrangements 8-27, wherein the formulation further comprises sucrose at a concentration of about 85 mM.

29. The antibody formulation of any one of arrangements 8-27, wherein the formulation further comprises sucrose at a concentration of about 150 mM.

30. The antibody formulation of any one of arrangements 8-27, wherein the formulation further comprises sucrose at a concentration of about 200 mM.

31. An antibody formulation comprising citrate phosphate and an antibody, wherein the antibody targets P. gingivalis.

32. The antibody formulation of arrangement 31, wherein the antibody comprises at least one, two, three, four, five, or all six of the CDR regions of SEQ ID NOS: 1-2.

33. The antibody formulation of any one of arrangements 31 or 32, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

34. An antibody formulation comprising citrate phosphate and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

35. The antibody formulation of arrangement 34, wherein the antibody targets P. gingivalis.

36. The antibody formulation of any one of arrangements 34 or 35, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

37. The antibody formulation of any one of arrangements 34-36, wherein the antibody comprises a sequence that has at least 75%, 79%, or 80% identity to SEQ ID NO: 1 and a sequence that has at least 75%, 79%, or 80% identity to SEQ ID NO: 2.

38. The antibody formulation of any one of arrangements 34-37, wherein the antibody is present at a concentration of about 0.1 mg/mL.

39. The antibody formulation of any one of arrangements 34-38, wherein the citrate phosphate is present at a concentration of about 1 to about 20 mM.

40. The antibody formulation of any one of arrangements 34-39, wherein the citrate phosphate is present at a concentration of about 10 mM.

41. The antibody formulation of any one of arrangements 34-40, wherein the formulation has a pH of about 4 to about 8.

42. The antibody formulation of any one of arrangements 34-41, wherein the formulation has a pH of 6.

43. The antibody formulation of any one of arrangements 34-42, wherein the formulation has a pH of 5.8.

44. The antibody formulation of any one of arrangements 34-43, wherein the formulation further comprises saline (sodium chloride) at a concentration of about 0.1-2.0% weight/volume.

45. The antibody formulation of any one of arrangements 34-44, wherein the formulation further comprises saline (sodium chloride) at a concentration of about 0.9% weight/volume.

46. The antibody formulation of any one of arrangements 34-45, wherein the formulation further comprises histidine at a concentration of about 1 to about 40 mM.

47. The antibody formulation of any one of arrangements 34-46, wherein the formulation further comprises histidine at a concentration of 20 mM.

48. The antibody formulation of any one of arrangements 34-47, wherein the formulation further comprises acetic acid (acetate) at a concentration of about 1 to about 20 mM.

49. The antibody formulation of any one of arrangements 34-48, wherein the formulation further comprises acetic acid (acetate) at a concentration of about 10 mM.

50. The antibody formulation of any one of arrangements 34-49, wherein the formulation further comprises Poloxamer P188 at a concentration of about 0.001-0.1% weight/volume.

51. The antibody formulation of any one of arrangements 34-50, wherein the formulation further comprises Poloxamer P188 at a concentration of about 0.01% weight/volume.

52. The antibody formulation of any one of arrangements 34-51, wherein the formulation further comprises sucrose at a concentration of about 85 to 200 mM.

53. The antibody formulation of any one of arrangements 34-52, wherein the formulation further comprises sucrose at a concentration of about 85 mM.

54. The antibody formulation of any one of arrangements 34-52, wherein the formulation further comprises sucrose at a concentration of about 150 mM.

55. The antibody formulation of any one of arrangements 34-52, wherein the formulation further comprises sucrose at a concentration of about 200 mM.

56. An antibody formulation comprising acetic acid (acetate) and an antibody, wherein the antibody targets P. gingivalis.

57. The antibody formulation of arrangement 56, wherein the antibody comprises at least one, two, three, four, five, or all six of the CDR regions of SEQ ID NOS: 1-2.

58. The antibody formulation of any one of arrangements 56 or 57, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

59. An antibody formulation comprising acetic acid (acetate) and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

60. The antibody formulation of arrangement 59, wherein the antibody targets P. gingivalis.

61. The antibody formulation of any one of arrangements 59 or 60, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

62. The antibody formulation of any one of arrangements 59-61, wherein the antibody comprises a sequence that has at least 75%, 79%, or 80% identity to SEQ ID NO: 1 and a sequence that has at least 75%, 79%, or 80% identity to SEQ ID NO: 2.

63. The antibody formulation of any one of arrangements 59-62, wherein the antibody is present at a concentration of about 0.1 mg/mL.

64. The antibody formulation of any one of arrangements 59-63, wherein the acetic acid (acetate) is present at a concentration of about 1 to about 20 mM.

65. The antibody formulation of any one of arrangements 59-64, wherein the acetic acid (acetate) is present at a concentration of about 10 mM.

66. The antibody formulation of any one of arrangements 59-65, wherein the formulation has a pH of about 4 to about 8.

67. The antibody formulation of any one of arrangements 59-66, wherein the formulation has a pH of 5.

68. The antibody formulation of any one of arrangements 59-67, wherein the formulation further comprises saline (sodium chloride) at a concentration of about 0.1-2.0% weight/volume.

69. The antibody formulation of any one of arrangements 59-68, wherein the formulation further comprises saline (sodium chloride) at a concentration of about 0.9% weight/volume.

70. The antibody formulation of any one of arrangements 59-69, wherein the formulation further comprises histidine at a concentration of about 1 to about 40 mM.

71. The antibody formulation of any one of arrangements 59-70, wherein the formulation further comprises histidine at a concentration of 20 mM.

72. The antibody formulation of any one of arrangements 59-71, wherein the formulation further comprises citrate phosphate at a concentration of about 1 to about 20 mM.

73. The antibody formulation of any one of arrangements 59-72, wherein the formulation further comprises citrate phosphate at a concentration of about 10 mM.

74. The antibody formulation of any one of arrangements 59-73, wherein the formulation further comprises Poloxamer P188 at a concentration of about 0.001-0.1% weight/volume.

75. The antibody formulation of any one of arrangements 59-74, wherein the formulation further comprises Poloxamer P188 at a concentration of about 0.01% weight/volume.

76. The antibody formulation of any one of arrangements 59-75, wherein the formulation further comprises sucrose at a concentration of about 85 to 200 mM.

77. The antibody formulation of any one of arrangements 59-76, wherein the formulation further comprises sucrose at a concentration of about 85 mM.

78. The antibody formulation of any one of arrangements 59-76, wherein the formulation further comprises sucrose at a concentration of about 150 mM.

79. The antibody formulation of any one of arrangements 59-76, wherein the formulation further comprises sucrose at a concentration of about 200 mM.

80 An antibody formulation comprising a Poloxamer P188 and an antibody, wherein the antibody targets P. gingivalis.

81. The antibody formulation of arrangement 80, wherein the antibody comprises at least one, two, three, four, five, or all six of the CDR regions of SEQ ID NOS: 1-2.

82. The antibody formulation of any one of arrangements 80 or 81, wherein the antibody comprises a VH that has at least 75%, 79%, 80%, 90% or 100% identity to a VH within SEQ ID NO: 1 and a VL that has at least 75%, 79%, 80%, 90% or 100% identity to a VL within of SEQ ID NO: 2.

83. An antibody formulation comprising Poloxamer P188 and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

84. The antibody formulation of arrangement 83, wherein the antibody targets P. gingivalis.

85. The antibody formulation of any one of arrangements 83 or 84, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

86. The antibody formulation of any one of arrangements 83-85, wherein the antibody comprises a sequence that has at least 75%, 79%, or 80% identity to SEQ ID NO: 1 and a sequence that has at least 75%, 79%, or 80% identity to SEQ ID NO: 2.

87. The antibody formulation of any one of arrangements 83-86, wherein the antibody is present at a concentration of about 0.1 mg/mL.

88. The antibody formulation of any one of arrangements 83-87, wherein the Poloxamer P188 is present at a concentration of about 0.001-0.1% weight/volume.

89. The antibody formulation of any one of arrangements 83-88, wherein the Poloxamer P188 is present at a concentration of about 0.01% weight/volume.

90. The antibody formulation of any one of arrangements 83-89, wherein the formulation has a pH of about 4 to about 8.

91. The antibody formulation of any one of arrangements 83-90, wherein the formulation further comprises saline (sodium chloride) at a concentration of about 0.1-2.0% weight/volume.

92. The antibody formulation of any one of arrangements 83-91, wherein the formulation further comprises saline (sodium chloride) at a concentration of about 0.9% weight/volume.

93. The antibody formulation of any one of arrangements 83-92, wherein the formulation further comprises histidine at a concentration of about 1 to about 40 mM.

94. The antibody formulation of any one of arrangements 83-93, wherein the formulation further comprises histidine at a concentration of 20 mM.

95. The antibody formulation of any one of arrangements 83-94, wherein the formulation further comprises citrate phosphate at a concentration of about 1 to about 20 mM.

96. The antibody formulation of any one of arrangements 83-95, wherein the formulation further comprises citrate phosphate at a concentration of about 10 mM.

97. The antibody formulation of any one of arrangements 83-96, wherein the formulation further comprises acetic acid (acetate) at a concentration of about 1 to about 20 mM.

98. The antibody formulation of any one of arrangements 83-97, wherein the formulation further comprises acetic acid (acetate) at a concentration of about 10 mM.

99. The antibody formulation of any one of arrangements 83-98, wherein the formulation further comprises sucrose at a concentration of about 85 to 200 mM.

100. The antibody formulation of any one of arrangements 83-99, wherein the formulation further comprises sucrose at a concentration of about 85 mM.

101. The antibody formulation of any one of arrangements 83-99, wherein the formulation further comprises sucrose at a concentration of about 150 mM.

102. The antibody formulation of any one of arrangements 83-99, wherein the formulation further comprises sucrose at a concentration of about 200 mM.

103. An antibody formulation comprising a sucrose and an antibody, wherein the antibody targets P. gingivalis.

104. The antibody formulation of arrangement 103, wherein the antibody comprises at least one, two, three, four, five, or all six of the CDR regions of SEQ ID NOS: 1-2.

105. The antibody formulation of any one of arrangements 103 or 104, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

106. An antibody formulation comprising a sucrose and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

107. The antibody formulation of arrangement 106, wherein the antibody targets P. gingivalis.

108. The antibody formulation of any one of arrangements 106 or 107, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

109. The antibody formulation of any one of arrangements 106-108, wherein the antibody comprises a sequence that has at least 75%, 79%, or 80% identity to SEQ ID NO: 1 and a sequence that has at least 75%, 79%, or 80% identity to SEQ ID NO: 2.

110. The antibody formulation of any one of arrangements 106-109, wherein the antibody is present at a concentration of about 0.1 mg/mL.

111. The antibody formulation of any one of arrangements 106-110, wherein the sucrose is present at a concentration of about 85 to 200 mM.

112. The antibody formulation of any one of arrangements 106-111, wherein the sucrose is present at a concentration of about 85 mM.

113. The antibody formulation of any one of arrangements 106-111, wherein the sucrose is present at a concentration of about 150 mM.

114. The antibody formulation of any one of arrangements 106-111, wherein the sucrose is present at a concentration of about 200 mM.

115. The antibody formulation of any one of arrangements 106-114, wherein the formulation has a pH of about 4 to about 8.

116. The antibody formulation of any one of arrangements 106-115, wherein the formulation further comprises saline (sodium chloride) at a concentration of about 0.1-2.0% weight/volume.

117. The antibody formulation of any one of arrangements 106-116, wherein the formulation further comprises saline (sodium chloride) at a concentration of about 0.9% weight/volume.

118. The antibody formulation of any one of arrangements 106-117, wherein the formulation further comprises histidine at a concentration of about 1 to about 40 mM.

119. The antibody formulation of any one of arrangements 106-118, wherein the formulation further comprises histidine at a concentration of 20 mM.

120 The antibody formulation of any one of arrangements 106-119, wherein the formulation further comprises citrate phosphate at a concentration of about 1 to about 20 mM.

121. The antibody formulation of any one of arrangements 106-120, wherein the formulation further comprises citrate phosphate at a concentration of about 10 mM.

122. The antibody formulation of any one of arrangements 106-121, wherein the formulation further comprises acetic acid (acetate) at a concentration of about 1 to about 20 mM.

123. The antibody formulation of any one of arrangements 106-122, wherein the formulation further comprises acetic acid (acetate) at a concentration of about 10 mM.

124. The antibody formulation of any one of arrangements 106-123, wherein the formulation further comprises Poloxamer P188 at a concentration of about 0.001-0.1% weight/volume.

125. The antibody formulation of any one of arrangements 106-124, wherein the formulation further comprises Poloxamer P188 at a concentration of about 0.01% weight/volume.

126. An antibody formulation comprising saline, sucrose, Poloxamer P188, and an antibody, wherein the antibody targets P. gingivalis.

127. The antibody formulation of arrangement 126, wherein the antibody comprises at least one, two, three, four, five, or all six of the CDR regions of SEQ ID NOS: 1-2.

128. The antibody formulation of any one of arrangements 126 or 127, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

129. An antibody formulation comprising saline, sucrose, Poloxamer P188, and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

130. The antibody formulation of arrangement 129, wherein the antibody targets P. gingivalis.

131. The antibody formulation of any one of arrangements 129 or 130, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

132. The antibody formulation of any one of arrangements 129-131, wherein the antibody comprises a sequence that has at least 75%, 79%, or 80% identity to SEQ ID NO: 1 and a sequence that has at least 75%, 79%, or 80% identity to SEQ ID NO: 2.

133. The antibody formulation of any one of arrangements 129-132, wherein saline is present at a concentration of about 0.1-2.0% weight/volume.

134. The antibody formulation of any one of arrangements 129-133, wherein saline is present at a concentration of about 0.9% weight/volume.

135. An antibody formulation comprising histidine, sucrose, Poloxamer P188, and an antibody, wherein the antibody targets P. gingivalis.

136. The antibody formulation of arrangement 135, wherein the antibody comprises at least one, two, three, four, five, or all six of the CDR regions of SEQ ID NOS: 1-2.

137. The antibody formulation of any one of arrangements 135 or 136, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

138. An antibody formulation comprising histidine, sucrose, Poloxamer P188, and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

139. The antibody formulation of arrangement 138, wherein the antibody targets P. gingivalis.

140. The antibody formulation of any one of arrangements 138 or 139, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

141. The antibody formulation of any one of arrangements 138-140, wherein the antibody comprises a sequence that has at least 75%, 79%, or 80% identity to SEQ ID NO: 1 and a sequence that has at least 75%, 79%, or 80% identity to SEQ ID NO: 2.

142. The antibody formulation of any one of arrangements 138-141, wherein histidine is present at a concentration of about 1 to about 40 mM.

143. The antibody formulation of any one of arrangements 138-142, wherein histidine is present at a concentration of about 20 mM.

144. The antibody formulation of any one of arrangements 138-143, wherein the formulation has a pH of about 4 to about 8.

145. The antibody formulation of any one of arrangements 138-144, wherein the formulation has a pH of 6.

146. The antibody formulation of any one of arrangements 138-145, wherein the formulation has a pH of 5.8.

147. The antibody formulation of any one of arrangements 138-146, wherein the Poloxamer P188 is present at a concentration of about 0.001-0.1% weight/volume.

148. The antibody formulation of any one of arrangements 138-147, wherein the Poloxamer P188 is present at a concentration of about 0.01% weight/volume.

149. The antibody formulation of any one of arrangements 138-148, wherein the sucrose is present at a concentration of about 85 to 200 mM.

150. The antibody formulation of any one of arrangements 138-149, wherein the sucrose is present at a concentration of about 85 mM.

151. The antibody formulation of any one of arrangements 138-149, wherein the sucrose is present at a concentration of about 150 mM.

152. The antibody formulation of any one of arrangements 138-149, wherein the sucrose is present at a concentration of about 200 mM.

153. The antibody formulation of any one of the proceeding arrangements, wherein the antibody is KB001.

154. A method of storing an antibody, wherein the method comprises mixing the antibody with an antibody formulations comprising any one of the formulations of arrangements 1-153.

155. A use of the any one of the antibody formulations of arrangements 1-153 as a storage formulation to enhance the stability of an at least one antibody or antigen binding molecule.

156. The method or use of any one of arrangements 154 or 155, wherein the antibody formulation is stored at room temperature (about 20-29° C.); most preferably stored at about 25° C.

157. The method or use of any one of arrangements 154-156, wherein the antibody formulation is stored at below room temperature; preferably stored between about 20 to about −80° C.; most preferably stored at about 5° C.

158. The method or use of any one of arrangements 154-156, wherein the antibody formulation is stored at above room temperature; preferably stored between about 30 to about 80° C.; most preferably stored at about 40° C.

159. The method or use of any one of arrangements 154-158, wherein the antibody formulation is stored as a solid and/or frozen.

160. The method or use of any one of arrangements 154-158, wherein the antibody formulation is stored as a liquid.

161. The antibody formulation or method or use of any one of the preceding arrangements, wherein the antibody comprises the heavy chain of SEQ ID NO: 1 and the light chain of SEQ ID NO: 2.

The technology described herein is further illustrated by the following arrangements which in no way should be construed as being further limiting.

### EXAMPLES

### Example 1: Preparation of Formulations

In the following non-limiting example, a formulation will be prepared for enhanced stability and delivery of an ABM. In this example, the ABM is KB001. In an alternative example, the ABM may be ABM disclosed in the present application. This formulation will be formulated at a final pH of 6.0. In an alternative example, the formulation may have a final pH that is any value between about 4.0 and about 7.0. The formulation will be formulated with 20 mM Histidine/Histidine HCl. In an alternative example, the formulation may comprise one or more from the group consisting of: sodium chloride, histidine, histidine HCl, L-histidine, L-histidine HCl, citrate, phosphate, acetic acid/acetate, sucrose, and poloxamer P188. In a further alternative example, the formulation may comprise one or more from the group consisting of: 9.00 g/L of Sodium Chloride, saline at 0.9% weight/volume, 3.10 g/L of L-Histidine, 4.18 g/L of L-Histidine HCl, 1.78 g/L of disodium hydrogen phosphate dihydrate, 1.92 g/L of citric acid monohydrate, 10 mM Citrate Phosphate, 0.96 g/L of Sodium acetate, 0.27 g/L of Acetic acid, 10 mM Acetate, 0.10 g/L of Poloxamer P188, 0.01% weight/volume Poloxamer P188, about 29.09 g/L (85 mM) sucrose, about 51.35 g/L (150 mM) sucrose, about 68.46 g/L (200 mM) sucrose, or any concentration that is between about 85-200 mM sucrose.

### Example 2: Excipient Screening and Short-Term Stability Studies

The long-term stability of the monoclonal antibody KB001 in various formulations was screened at 5±3° C., 25±2° C. (60%±5% relative humidity, or “RH”), and 40±2° C. (75%±5% RH) temperature conditions over 3 months.

The excipient screening study disclosed herein was performed to evaluate excipients that impart shelf-life stability to the antibody in liquid formulations. The stability of the antibody was assessed at 7 day, 14 day, 1 month, 2 month, and 3 month time points. The physico-chemical stability of the antibody was evaluated using various analytical techniques. Appropriate excipients that were chosen for the final formulation were based on the results of these studies.

### Materials

The details of the antibody sample, list of equipment, list of materials, and consumables are mentioned in Tables 1, 2, 3, and 4, respectively.

TABLE 1
Details of the sample

| | Sample | Source | Antibody |
|---|---|---|---|
| 1 | mAb | Drug substance in saline (0.9% w/v) | KB001 |

TABLE 2
List of equipment

| | Instrument/Equipment | Instrument/Equipment ID |
|---|---|---|
| 1 | Stability chamber (5 ± 3° C.) | BPD/FDB/REF/001 |
| 2 | Stability chamber (25 ± 2° C.) | S17STC-LI-002 |
| | (60% ± 5% RH) | |
| 3 | Stability chamber (40 ± 2° C.) | S17STC-LI-007 |
| | (75% ± 5% RH) | |
| 4 | Electronic balance | BPD/ADB/WBL/02 |
| 5 | Bio-safety cabinet | BPD/BFF/BSC/001 |
| 6 | Centrifuge | BPD/DSB/CEN/001 |
| 7 | pH meter | BPD/ADB/PCM/001 |
| 8 | Pipette | BPD/FDB/PPT/003 |
| | | BPD/FDB/PPT/004 |
| | | BPD/FDB/PPT/007 |
| | | BPD/DPD/PPT/017 |

TABLE 3
List of materials

| | Chemicals | Grade | Make | Cat. No. |
|---|---|---|---|---|
| 1 | L-Histidine | Ph. Eur. | JT Baker | A1341.1000 |
| 2 | L-Histidine mono hydrochloride | Ph. Eur. | JT Baker | A1591.0500 |
| 3 | Hydrochloric acid | Ph. Eur. | JT Baker | 30024.324 |
| 4 | Poloxamer P188 | Ph. Eur. | Sigma | K4894-500G |
| 5 | Sucrose | Ph. Eur. | JT Baker | 4074-01 |
| 6 | Sodium chloride | Ph. Eur. | JT Baker | 5640-500 |

TABLE 4
List of consumables

| | Consumables | Make | Cat. No. |
|---|---|---|---|
| 1 | Filtration centrifuge tubes | Merck Millipore | UFC903096 |
| 2 | Pre-sterilized filtration units | Merck Millipore | S2GVUOSRE |
| 3 | Glass vials (10 R) | Schott Kaisha | 5000008394 |
| 4 | 0.22 μ filters (PES) | Pal Life Sciences | MCPM02C67 |
| 5 | Centrifuge tubes | Corning | 430791 |
| 6 | Rubber stoppers | West Pharmaceuticals | 19700022 |
| 7 | Flip-off seals | West Pharmaceuticals | 54203634 |

The drug substance (KB001) was buffer exchanged with the respective solutions and prepared as per standard protocol. The concentration of protein in the respective formulations after buffer exchange was determined using Solo VPE (Table 5). The formulations (ES1 to ES6) were prepared as per the composition mentioned in Table 6 and filtered using sterile 0.22μ syringe filters (PES). The final formulations were filled (9.0 mL) into a 10 R Type-I ready to use glass vial aseptically under the bio-safety cabinet, then stoppered and crimped. The stability samples were pulled out at respective time points (Table 7) and submitted for analysis as mentioned in Table 8. The sample pull out details from the S17 facility were recorded in annexure No. 3 of stability sample inception and pull-out register of SOP, S17/STC/SOP/004.

TABLE S
Concentration of antibody after solution exchange

| | Formulation | Protein Concentration |
|---|---|---|
| 1 | Saline (0.9% w/v) | 7.2 mg/mL |
| 2 | 20 mM Histidine/Histidine HCl, pH 6.0 | 8.5 mg/mL |

TABLE 6
Sample names with formulation systems and excipients

| Solution | Solution | Excipient 1 | Excipient 2 | Concentration |
|---|---|---|---|---|
| ES 1 | Saline (0.9% w/v) | 85 mM sucrose | 0.01% Poloxamer | 0.10 mg/mL |
| | | | P188 | |
| ES 2 | Saline (0.9% w/v) | 150 mM sucrose | 0.01% Poloxamer | 0.12 mg/mL |
| | | | P188 | |
| ES 3 | Saline (0.9% w/v) | 200 mM sucrose | 0.01% Poloxamer | 0.12 mg/mL |
| | | | P188 | |
| ES 4 | 20 mM | 85 mM sucrose | 0.01% Poloxamer | 0.10 mg/mL |
| | Histidine/Histidine HCl | | P188 | |
| ES 5 | 20 mM | 150 mM sucrose | 0.01% Poloxamer | 0.10 mg/mL |
| | Histidine/Histidine HCl | | P188 | |
| ES 6 | 20 mM | 200 mM sucrose | 0.01% Poloxamer | 0.10 mg/mL |
| | Histidine/Histidine HCl | | P188 | |

TABLE 7
Sample pull out plan for stability study and vial information

| | Time point | | | | | | Contingency | Total |
|---|---|---|---|---|---|---|---|---|
| Temperature | Initial | 7 D | 14 D | 1 M | 2 M | 3 M | (vials) | vials |
| 5 ± 3° C. | 1* | NA | 1 | 1 | 1 | 1 | 2 | 6 |
| 25 ± 2° C./ | | NA | 1 | 1 | 1 | 1 | 2 | 6 |
| 60% ± 5% RH | | | | | | | | |
| 40 ± 2° C./ | | 1 | 1 | 1 | NA | NA | 2 | 5 |
| 75% ± 5% RH | | | | | | | | |

*Initial vials are not included in the total vials as it was submitted for analysis on the day of stability inception.

Note: The sample pull out plan is applicable to all samples (ES1 to ES6).

TABLE 8
List of analytical tests

| | | Sample requirement | |
|---|---|---|---|
| | | for analysis at each | |
| | Analytical Tests | time point (mL) | AMP No. |
| 1 | Visual appearance | 1.0 | PT-18/0088 |
| 2 | Concentration by UV 280 | 0.5 | PT-20/0048 |
| 3 | pH at 25° C. | 0.5 | PT-18/0089 |
| 4 | NR-CE SDS | 1.5 | PT-20/0053 |
| 5 | R-CE SDS | 1.0 | PT-20/0060 |
| 6 | SEC-HPLC | 1.0 | PT-20/0047 |
| 7 | IEX-HPLC | 1.0 | PT-20/0055 |
| 8 | Osmolality | 0.5 | PT-19/0012 |
| 9 | Sub-visible particle | 1.0 | Pharmacopoeia |
| | | | test |
| 10 | Potency by ELISA* | 0.5 | NA |

*ELISA test conducted for T = 0 day, T = 7 day, T = 1 month and T = 3 month samples only.

### Results and Conclusion

The results of the study were as shown in Tables 9-18.

TABLE 9
pH

| Sample | | 7 D | 14 D | 14 D | 14 D | 1 M | 1 M | 1 M | 2 M | 2 M | 3 M | 3 M |
|---|---|---|---|---|---|---|---|---|---|---|---|---|
| code | 0 D | 40° C. | 5° C. | 25° C. | 40° C. | 5° C. | 25° C. | 40° C. | 5° C. | 25° C. | 5° C. | 25° C. |
| ES1 | 5.2 | 5.7 | 5.4 | 5.4 | 5.5 | 5.7 | 5.3 | 5.3 | 5.6 | 5.8 | 5.9 | 5.7 |
| ES2 | 5.4 | 5.8 | 5.2 | 5.6 | 5.2 | 5.6 | 5.5 | 5.6 | 5.8 | 5.7 | 5.6 | 5.7 |
| ES3 | 5.4 | 6.2 | 5.6 | 5.6 | 5.7 | 6.0 | 6.0 | 5.6 | 5.9 | 5.7 | 5.4 | 5.8 |
| ES4 | 6.0 | 6.1 | 6.1 | 6.1 | 6.1 | 6.1 | 6.1 | 6.0 | 6.0 | 6.0 | 5.8 | 5.8 |
| ES5 | 6.0 | 6.1 | 6.0 | 6.1 | 6.1 | 6.2 | 6.1 | 6.0 | 5.9 | 6.0 | 5.8 | 5.8 |
| ES6 | 6.0 | 6.0 | 6.1 | 6.1 | 6.1 | 6.1 | 6.0 | 6.0 | 6.0 | 6.0 | 5.8 | 5.8 |

TABLE 10
Visual appearance

| Sample | | 7 D | 14 D | 14 D | 14 D | 1 M | 1 M | 1 M | 2 M | 2 M | 3 M | 3 M |
|---|---|---|---|---|---|---|---|---|---|---|---|---|
| code | 0 D | 40° C. | 5° C. | 25° C. | 40° C. | 5° C. | 25° C. | 40° C. | 5° C. | 25° C. | 5° C. | 25° C. |
| ES1 | * | * | * | * | * | * | * | * | * | * | * | * |
| ES2 | * | * | * | * | * | * | * | * | * | * | * | * |
| ES3 | * | * | * | * | * | * | * | * | * | * | * | * |
| ES4 | * | * | * | * | * | * | * | * | * | * | * | * |
| ES5 | * | * | * | * | * | * | * | * | * | * | * | * |
| ES6 | * | * | * | * | * | * | * | * | * | * | * | * |

* Free of visible particles, colourless, and clear.

TABLE 11
Concentration (mg/mL)

| Sample | | 7 D | 14 D | 14 D | 14 D | 1 M | 1 M | 1 M | 2 M | 2 M | 3 M | 3 M |
|---|---|---|---|---|---|---|---|---|---|---|---|---|
| code | 0 D | 40° C. | 5° C. | 25° C. | 40° C. | 5° C. | 25° C. | 40° C. | 5° C. | 25° C. | 5° C. | 25° C. |
| ES1 | 0.11 | 0.11 | 0.10 | 0.10 | 0.11 | 0.11 | 0.08 | 0.11 | 0.11 | 0.12 | 0.11 | 0.11 |
| ES2 | 0.12 | 0.11 | 0.11 | 0.10 | 0.11 | 0.11 | 0.11 | 0.11 | 0.12 | 0.12 | 0.12 | 0.12 |
| ES3 | 0.12 | 0.11 | 0.11 | 0.11 | 0.11 | 0.11 | 0.11 | 0.11 | 0.13 | 0.12 | 0.14 | 0.12 |
| ES4 | 0.10 | 0.09 | 0.09 | 0.09 | 0.10 | 0.09 | 0.11 | 0.09 | 0.10 | 0.11 | 0.10 | 0.11 |
| ES5 | 0.10 | 0.09 | 0.09 | 0.09 | 0.09 | 0.09 | 0.09 | 0.10 | 0.08 | 0.11 | 0.10 | 0.11 |
| ES6 | 0.10 | 0.10 | 0.10 | 0.10 | 0.10 | 0.08 | 0.10 | 0.10 | 0.11 | 0.11 | 0.11 | 0.12 |

TABLE 12
Osmolality (mOsmol/kg)

| Sample | | 7 D | 14 D | 14 D | 14 D | 1 M | 1 M | 1 M | 2 M | 2 M | 3 M | 3 M |
|---|---|---|---|---|---|---|---|---|---|---|---|---|
| code | 0 D | 40° C. | 5° C. | 25° C. | 40° C. | 5° C. | 25° C. | 40° C. | 5° C. | 25° C. | 5° C. | 25° C. |
| ES1 | 354 | 359 | 349 | 358 | 358 | 357 | 357 | 357 | 359 | 360 | 364 | 365 |
| ES2 | 409 | 417 | 417 | 417 | 415 | 416 | 418 | 418 | 419 | 422 | 421 | 422 |
| ES3 | 457 | 464 | 464 | 464 | 460 | 459 | 461 | 463 | 466 | 468 | 469 | 467 |
| ES4 | 114 | 116 | 111 | 115 | 118 | 114 | 115 | 117 | 117 | 115 | 114 | 116 |
| ES5 | 178 | 181 | 177 | 176 | 180 | 176 | 177 | 179 | 181 | 181 | 180 | 180 |
| ES6 | 227 | 234 | 229 | 233 | 235 | 229 | 232 | 233 | 233 | 234 | 234 | 236 |

TABLE 13
UPSEC

| Sample | | | 7 D | 14 D | 14 D | 14 D | 1 M | 1 M | 1 M | 2 M | 2 M | 3 M | 3 M |
|---|---|---|---|---|---|---|---|---|---|---|---|---|---|
| code | Attribute | 0 D | 40° C. | 5° C. | 25° C. | 40° C. | 5° C. | 25° C. | 40° C. | 5° C. | 25° C. | 5° C. | 25° C. |
| ES1 | % Monomer | 99.1 | 99.3 | 99.3 | 99.4 | 99.4 | 99.3 | 99.3 | 99.3 | 99.3 | 99.3 | 99.3 | 99.3 |
| | % HMMS | 0.9 | 0.7 | 0.7 | 0.6 | 0.6 | 0.7 | 0.7 | 0.7 | 0.7 | 0.7 | 0.7 | 0.7 |
| ES2 | % Monomer | 99.0 | 98.8 | 99.2 | 99.3 | 98.9 | 99.2 | 99.3 | 99.3 | 99.3 | 99.4 | 99.3 | 99.3 |
| | % HMMS | 1.0 | 1.2 | 0.8 | 0.7 | 1.1 | 0.8 | 0.7 | 0.7 | 0.7 | 0.6 | 0.7 | 0.7 |
| ES3 | % Monomer | 99.0 | 99.3 | 99.1 | 99.2 | 99.3 | 99.2 | 99.3 | 99.3 | 99.3 | 99.4 | 99.3 | 99.2 |
| | % HMMS | 1.0 | 0.7 | 1.0 | 0.8 | 0.7 | 0.8 | 0.7 | 0.7 | 0.7 | 0.6 | 0.7 | 0.8 |
| ES4 | % Monomer | 99.9 | 99.9 | 99.9 | 99.9 | 99.9 | 99.9 | 99.9 | 99.9 | 99.9 | 99.9 | 99.9 | 99.9 |
| | % HMMS | 0.1 | 0.1 | 0.1 | 0.1 | 0.1 | 0.1 | 0.1 | 0.1 | 0.1 | 0.1 | 0.1 | 0.1 |
| ES5 | % Monomer | 99.8 | 99.9 | 99.7 | 99.7 | 99.9 | 99.9 | 99.8 | 99.8 | 99.9 | 99.9 | 99.9 | 99.9 |
| | % HMMS | 0.2 | 0.1 | 0.3 | 0.3 | 0.1 | 0.1 | 0.1 | 0.2 | 0.1 | 0.2 | 0.1 | 0.1 |
| ES6 | % Monomer | 99.5 | 99.8 | 99.9 | 99.9 | 99.9 | 99.5 | 99.9 | 99.8 | 99.8 | 99.5 | 99.8 | 99.8 |
| | % HMMS | 0.2 | 0.2 | 0.1 | 0.1 | 0.2 | 0.2 | 0.1 | 0.2 | 0.2 | 0.2 | 0.2 | 0.2 |

TABLE 14
iEX

| Sample | | | 7 D | 14 D | 14 D | 14 D | 1 M | 1 M | 1 M | 2 M | 2 M | 3 M | 3 M |
|---|---|---|---|---|---|---|---|---|---|---|---|---|---|
| code | Attribute | 0 D | 40° C. | 5° C. | 25° C. | 40° C. | 5° C. | 25° C. | 40° C. | 5° C. | 25° C. | 5° C. | 25° C. |
| ES1 | % Main peak | 7.1 | 5.6 | 8.7 | 7.7 | 7.3 | 7.1 | 6.8 | 5.8 | 7.5 | 6.8 | 7.3 | 6.9 |
| | % Acidic | 5.4 | 3.7 | 8.5 | 9.3 | 13.1 | 9.9 | 12.1 | 19.6 | 9.7 | 12.7 | 9.0 | 14.5 |
| | % Basic | 57.5 | 90.7 | 82.8 | 83.1 | 75.6 | 53.0 | 81.2 | 74.7 | 62.9 | 80.6 | 83.7 | 75.6 |
| ES2 | % Main peak | 7.0 | 5.7 | 8.6 | 7.8 | 7.4 | 7.1 | 6.8 | 5.9 | 7.5 | 6.9 | 7.5 | 7.1 |
| | % Acidic | 5.1 | 3.6 | 8.8 | 10.0 | 14.4 | 9.9 | 11.3 | 19.7 | 9.3 | 12.7 | 9.3 | 14.3 |
| | % Basic | 57.9 | 90.7 | 82.7 | 82.3 | 75.2 | 53.1 | 81.9 | 74.4 | 63.1 | 80.4 | 83.2 | 75.5 |
| ES3 | % Main peak | 7.0 | 5.5 | 7.7 | 7.9 | 7.4 | 7.3 | 6.8 | 5.1 | 7.5 | 6.9 | 7.3 | 6.8 |
| | % Acidic | 4.2 | 3.6 | 8.6 | 9.0 | 13.0 | 10.0 | 11.2 | 20.0 | 10.5 | 12.9 | 9.1 | 14.3 |
| | % Basic | 88.6 | 90.9 | 83.8 | 83.1 | 79.6 | 82.7 | 82.0 | 74.0 | 82.0 | 80.2 | 83.7 | 78.9 |
| ES4 | % Main peak | 8.3 | 7.7 | 10.1 | 8.8 | 7.2 | 8.3 | 7.1 | 5.8 | 8.5 | 6.3 | 7.8 | 4.5 |
| | % Acidic | 5.5 | 6.8 | 10.7 | 12.5 | 15.6 | 12.2 | 15.6 | 26.4 | 12.5 | 18.5 | 10.0 | 15.0 |
| | % Basic | 86.3 | 85.5 | 79.2 | 78.7 | 75.2 | 79.5 | 77.3 | 67.8 | 78.9 | 75.2 | 82.3 | 80.5 |
| ES5 | % Main peak | 8.3 | 7.6 | 9.6 | 8.7 | 7.4 | 8.3 | 7.1 | 5.5 | 8.6 | 6.0 | 3.7 | 4.1 |
| | % Acidic | 5.9 | 6.4 | 10.3 | 12.1 | 15.1 | 13.0 | 15.4 | 25.7 | 11.8 | 15.8 | 14.8 | 15.9 |
| | % Basic | 85.8 | 86.1 | 80.1 | 79.2 | 75.5 | 78.7 | 77.5 | 68.9 | 79.6 | 77.2 | 81.5 | 80.1 |
| ES6 | % Main peak | 8.4 | 4.2 | 9.8 | 9.3 | 7.4 | 8.3 | 7.5 | 5.8 | 8.7 | 6.5 | 5.0 | 5.4 |
| | % Acidic | 6.2 | 3.8 | 10.1 | 13.1 | 16.0 | 11.0 | 16.3 | 25.2 | 11.6 | 15.1 | 13.1 | 15.1 |
| | % Basic | 85.4 | 92.0 | 80.1 | 77.6 | 76.6 | 80.7 | 76.3 | 69.0 | 79.7 | 77.5 | 81.9 | 79.6 |

TABLE 15
NR-CGE

| Sample | | | 7 D | 14 D | 14 D | 14 D | 1 M | 1 M | 1 M | 2 M | 2 M | 3 M | 3 M |
|---|---|---|---|---|---|---|---|---|---|---|---|---|---|
| code | Attribute | 0 D | 40° C. | 5° C. | 25° C. | 40° C. | 5° C. | 25° C. | 40° C. | 5° C. | 25° C. | 5° C. | 25° C. |
| ES1 | % Monomer | 91.9 | 74.0 | 87.3 | 86.4 | 69.7 | 91.3 | 78.3 | 56.1 | 85.8 | 71.8 | 83.3 | 62.8 |
| | % LMMS | 8.1 | 9.7 | 12.7 | 13.6 | 6.3 | 8.7 | 6.3 | 8.5 | 14.2 | 8.2 | 16.7 | 16.7 |
| ES2 | % Monomer | 93.6 | 70.6 | 86.6 | 84.3 | 70.6 | 90.5 | 77.9 | 55.4 | 57.1 | 72.4 | 84.2 | 64.9 |
| | % LMMS | 6.4 | 13.6 | 13.4 | 15.7 | 6.3 | 9.5 | 5.4 | 7.2 | 12.9 | 7.5 | 15.8 | 14.2 |
| ES3 | % Monomer | 95.6 | 75.7 | 89.2 | 86.9 | 68.6 | 90.3 | 79.3 | 49.7 | 89.5 | 74.6 | 83.2 | 64.2 |
| | % LMMS | 4.4 | 6.0 | 10.8 | 13.1 | 6.5 | 9.7 | 6.0 | 9.4 | 10.4 | 6.8 | 16.8 | 15.8 |
| ES4 | % Monomer | 95.7 | 84.9 | 93.9 | 95.5 | 75.8 | 94.9 | 95.2 | 59.5 | 95.3 | 81.4 | 93.7 | 75.2 |
| | % LMMS | 4.3 | 5.0 | 6.1 | 4.5 | 5.2 | 5.1 | 4.9 | 3.3 | 4.7 | 4.5 | 6.3 | 5.6 |
| ES5 | % Monomer | 95.2 | 96.5 | 93.7 | 92.8 | 76.4 | 95.2 | 95.2 | 61.5 | 95.0 | 81.3 | 93.6 | 74.2 |
| | % LMMS | 4.8 | 3.5 | 6.3 | 7.2 | 4.4 | 4.9 | 4.8 | 3.2 | 5.0 | 4.4 | 6.4 | 6.3 |
| ES6 | % Monomer | 95.1 | 83.6 | 92.5 | 95.5 | 76.5 | 95.1 | 95.7 | 55.4 | 95.0 | 82.1 | 93.9 | 75.5 |
| | % LMMS | 4.9 | 4.9 | 7.5 | 4.4 | 4.5 | 4.9 | 4.3 | 3.3 | 5.0 | 4.1 | 6.2 | 5.3 |

TABLE 16
R-CGE

| Sample | | | 7 D | 14 D | 14 D | 14 D | 1 M | 1 M | 1 M | 2 M | 2 M | 3 M | 3 M |
|---|---|---|---|---|---|---|---|---|---|---|---|---|---|
| code | Attribute | 0 D | 40° C. | 5° C. | 25° C. | 40° C. | 5° C. | 25° C. | 40° C. | 5° C. | 25° C. | 5° C. | 25° C. |
| ES1 | % LC + HC | 91.0 | 86.0 | 97.6 | 98.0 | 98.0 | 94.5 | 97.4 | 96.0 | 95.9 | 94.2 | 95.3 | 94.4 |
| | % Fragments | 0.2 | 0.6 | 0.5 | 0.3 | 0.4 | 0.5 | 0.7 | 1.5 | NA | 0.7 | NA | 2.7 |
| | % Non-Reduced | 8.7 | 13.2 | 1.8 | 1.6 | 1.6 | 5.0 | 1.9 | 2.5 | 4.1 | 5.1 | 4.7 | 2.8 |
| ES2 | % LC + HC | 89.4 | 94.9 | 98.1 | 97.9 | 96.6 | 97.5 | 96.7 | 96.3 | 95.8 | 94.4 | 94.7 | 94.2 |
| | % Fragments | 0.7 | 1.4 | 0.3 | 0.6 | 0.9 | 0.4 | 0.7 | 2.2 | NA | 0.8 | NA | 2.5 |
| | % Non-Reduced | 10.0 | 3.8 | 1.6 | 1.4 | 2.4 | 2.2 | 2.8 | 1.5 | 4.2 | 4.8 | 5.3 | 3.3 |
| ES3 | % LC + HC | 96.0 | 95.3 | 97.8 | 98.9 | 97.8 | 96.8 | 94.3 | 95.7 | 95.6 | 95.2 | 95.0 | 95.1 |
| | % Fragments | 0.3 | 1.2 | 0.5 | 0.3 | 1.1 | 0.6 | 0.5 | 1.7 | NA | 1.2 | NA | 1.4 |
| | % Non-Reduced | 3.7 | 3.5 | 1.7 | 0.8 | 1.1 | 2.5 | 5.2 | 2.6 | 4.2 | 3.8 | 4.0 | 3.5 |
| ES4 | % LC + HC | 96.1 | 97.4 | 99.0 | 98.8 | 98.2 | 98.3 | 98.1 | 96.3 | 94.2 | 93.7 | 96.0 | 96.6 |
| | % Fragments | 0.3 | 0.8 | 0.2 | 0.3 | 0.7 | 0.1 | 0.2 | 1.8 | 0.4 | 0.7 | 0.6 | 1.0 |
| | % Non-Reduced | 3.6 | 1.6 | 0.9 | 0.9 | 1.2 | 1.6 | 1.7 | 1.8 | 5.5 | 5.7 | 3.4 | 2.3 |
| ES5 | % LC + HC | 95.2 | 98.0 | 99.1 | 98.7 | 98.5 | 98.8 | 97.9 | 96.8 | 97.7 | 96.2 | 98.4 | 96.4 |
| | % Fragments | 0.3 | 0.6 | 0.2 | 0.0 | 0.8 | 0.4 | 0.2 | 1.8 | 0.4 | 0.7 | 0.5 | 1.6 |
| | % Non-Reduced | 4.5 | 1.4 | 0.7 | 1.3 | 0.7 | 0.8 | 1.9 | 1.4 | 1.9 | 3.1 | 1.1 | 2.0 |
| ES6 | % LC + HC | 93.8 | 97.6 | 98.6 | 99.0 | 98.5 | 95.5 | 97.5 | 96.2 | 97.2 | 96.3 | 58.1 | 97.0 |
| | % Fragments | 0.3 | 0.5 | 0.2 | 0.3 | 0.7 | 0.4 | 0.5 | 1.5 | 0.4 | 0.5 | 0.6 | 1.1 |
| | % Non-Reduced | 5.9 | 1.8 | 1.2 | 0.7 | 0.9 | 1.2 | 2.0 | 2.5 | 2.4 | 3.1 | 1.3 | 1.9 |

TABLE 17
Sub-visible particles

| Sample | | | 7 D | 14 D | 14 D | 14 D | 1 M | 1 M | 1 M | 2 M | 2 M | 3 M | 3 M |
|---|---|---|---|---|---|---|---|---|---|---|---|---|---|
| code | Attribute | 0 D | 40° C. | 5° C. | 25° C. | 40° C. | 5° C. | 25° C. | 40° C. | 5° C. | 25° C. | 5° C. | 25° C. |
| ES1 | 10 μm | 405.0 | 42.0 | 592.0 | 1442.0 | 350.0 | 542.0 | 800.0 | 592.0 | 458.0 | 608.0 | 266.7 | 165.7 |
| | 25 μm | 17.0 | 0.0 | 50.0 | 133.0 | 17.0 | 33.0 | 50.0 | 33.0 | 50.0 | 17.0 | 83.3 | 50.0 |
| ES2 | 10 μm | 133.0 | 17.0 | 425.0 | 1533.0 | 500.0 | 100.0 | 442.0 | 392.0 | 575.0 | 150.0 | 33.3 | 83.3 |
| | 25 μm | 42.0 | 0.0 | 50.0 | 50.0 | 50.0 | 8.0 | 17.0 | 25.0 | 25.0 | 33.0 | 0.0 | 33.3 |
| ES3 | 10 μm | 192.0 | 50.0 | 250.0 | 567.0 | 258.0 | 217.0 | 250.0 | 400.0 | 167.0 | 392.0 | 316.7 | 50.0 |
| | 25 μm | 0.0 | 0.0 | 8.0 | 17.0 | 8.0 | 17.0 | 17.0 | 42.0 | 0.0 | 17.0 | 33.3 | 0.0 |
| ES4 | 10 μm | 250.0 | 150.0 | 308.0 | 475.0 | 192.0 | 267.0 | 125.0 | 567.0 | 342.0 | 242.0 | 116.7 | 50.0 |
| | 25 μm | 33.0 | 8.0 | 8.0 | 17.0 | 0.0 | 8.0 | 0.0 | 8.0 | 17.0 | 0.0 | 0.0 | 0.0 |
| ES5 | 10 μm | 267.0 | 142.0 | 142.0 | 475.0 | 217.0 | 342.0 | 300.0 | 533.0 | 367.0 | 167.0 | 183.3 | 166.7 |
| | 25 μm | 8.0 | 8.0 | 0.0 | 17.0 | 17.0 | 17.0 | 17.0 | 25.0 | 25.0 | 17.0 | 0.0 | 50.0 |
| ES6 | 10 μm | 275.0 | 50.0 | 250.0 | 667.0 | 158.0 | 417.0 | 392.0 | 283.0 | 617.0 | 433.0 | 183.3 | 133.3 |
| | 25 μm | 33.0 | 0.0 | 25.0 | 33.0 | 17.0 | 33.0 | 8.0 | 25.0 | 17.0 | 25.0 | 50.0 | 33.3 |

TABLE 18
Potency by ELISA

| | | 7 D | 1 M | 1 M | 3 M | 1 M | 3 M |
|---|---|---|---|---|---|---|---|
| Sample | 0 D | 40° C. | 40° C. | 25° C. | 25° C. | 5° C. | 5° C. |
| ES1 | 0.97 | 0.71 | 1.00 | 1.01 | 0.92 | 1.07 | 1.47 |
| ES2 | 1.00 | 0.70 | 0.87 | 1.06 | 1.02 | 1.09 | 1.15 |
| ES3 | 0.90 | 0.91 | 1.05 | 0.94 | 0.77 | 0.98 | 1.18 |
| ES4 | 0.97 | 0.92 | 0.93 | 1.01 | 0.73 | 1.04 | 1.33 |
| ES5 | 0.82 | 1.00 | 0.66 | 0.92 | 0.78 | 0.79 | 0.95 |
| ES6 | 0.91 | 0.98 | 0.65 | 0.99 | 0.66 | 1.00 | 0.95 |

The thermal stability of the antibody (0.1 mg/mL) formulated in saline (0.9% w/v) and L-histidine/Histidine HCl buffer containing sucrose (85 mM, 150 mM, and 200 mM), poloxamer P188 (0.01%) was studied. It was observed that the formulations were clear, colorless and free of visible particles at all temperature conditions. The sub-visible particle count of 10 μm and 25 μm particles are within the acceptable specification limit. There is no significant change in pH, concentration, or osmolality observed in all the formulations. It is observed that there is no significant difference observed in % HMMS by SEC in all the formulations. However, percent LMMS by NR-CGE was relatively stable in L-Histidine/L-Histidine HCl buffer compared to saline. The percent fragments by R-CGE were stable in all formulations. The % acidic variants by iEX were stable at 5+3° C. in all formulations. However, there was an increasing trend observed at higher temperatures: the percent acidic variants were comparatively stable in saline than L-Histidine/L-Histidine HCl buffer. The potency of the stability samples was analyzed in comparison to an internal reference standard, and showed that all formulations had a dose-dependent response.

### Example 3: Freeze-Thaw Stability Studies

The effect of formulation on the long-term stability of the antibody KB001 through freeze-thawing was screened. The antibody was added to the formulation at a final concentration of 0.1 mg/mL. The studies were conducted as “FT” (Freeze and Thaw) Slow and Fast stability studies at timepoints: initial, FT1, FT3, and FT5. Analytical tests for monitoring stability were pH, visual appearance, concentration, SEC, NR & R-CGE, and iEX. The formulation conditions and results were as depicted in Tables 19-24.

TABLE 19
Formulations

| | | Concentration of drug |
|---|---|---|
| Sample Code | Solution used | substance |
| BPD0854 FTS F1 | Saline (0.9% w/v) | 0.1 mg/mL |
| BPD0854 FTS F2 | 20 mM His./His. HCl, pH 6.0 | 0.1 mg/mL |
| BPD0854 FTS F3 | Saline (0.9% w/v) | 7.4 mg/mL |
| BPD0854 FTS F4 | 20 mM His./His. HCl, pH 6.0 | 5.5 mg/mL |

TABLE 20
pH

| Sample | | Slow FT cycle | | | Fast FT cycle | | | |
|---|---|---|---|---|---|---|---|---|
| code | Initial | 1 F/T | 3 F/T | 5 F/T | 1 F/T | 3 F/T | 5 F/T | Control |
| FTS F1 | 5.6 | 4.6 | 4.9 | 4.6 | 4.6 | 4.5 | 4.4 | 4.1 |
| FTS F2 | 6.0 | 6.0 | 6.1 | 6.0 | 6.1 | 6.0 | 6.1 | 6.0 |
| FTS F3 | 6.3 | 6.2 | 6.2 | 6.1 | 6.3 | 6.1 | 6.0 | 6.0 |
| FTS F4 | 6.0 | 6.0 | 6.1 | 6.0 | 6.1 | 6.0 | 6.1 | 6.0 |

TABLE 21
Concentration (mg/mL) as determined by
the UV 280, collected through Solo VPE

| Sample | | Slow FT cycle | | | Fast FT cycle | | | |
|---|---|---|---|---|---|---|---|---|
| code | Initial | 1 F/T | 3 F/T | 5 F/T | 1 F/T | 3 F/T | 5 F/T | Control |
| FTS F1 | 0.1 | 0.1 | 0.1 | 0.1 | 0.1 | 0.1 | 0.1 | 0.1 |
| FTS F2 | 0.1 | 0.1 | 0.1 | 0.1 | 0.7 | 0.1 | 0.1 | 0.1 |
| FTS F3 | 7.4 | 7.6 | 5.5 | 7.6 | 7.3 | 7.4 | 7.7 | 7.7 |
| FTS F4 | 5.5 | 5.5 | 5.7 | 5.6 | 5.4 | 5.4 | 5.4 | 5.4 |

TABLE 22
SEC

| Sample | | | Slow FT cycle | | | Fast FT cycle | | | |
|---|---|---|---|---|---|---|---|---|---|
| code | Attributes | Initial | 1 F/T | 3 F/T | 5 F/T | 1 F/T | 3 F/T | 5 F/T | Control |
| FTS F1 | % HMMS | 0.3 | 1.1 | 7.4 | 9.3 | 2.2 | 10.0 | 18.8 | 0.2 |
| | % Monomer | 99.7 | 98.9 | 92.6 | 90.7 | 97.8 | 90.0 | 81.2 | 99.8 |
| FTS F2 | % HMMS | 0.0 | 0.0 | 0.0 | 0.0 | 0.0 | 0.0 | 0.0 | 0.0 |
| | % Monomer | 100.0 | 100.0 | 100.0 | 100.0 | 100.0 | 100.0 | 100.0 | 100.0 |
| FTS F3 | % HMMS | 0.5 | 3.1 | 6.1 | 8.3 | 2.5 | 5.5 | 7.8 | 0.6 |
| | % Monomer | 99.5 | 98.2 | 93.9 | 91.7 | 97.5 | 94.5 | 92.2 | 99.4 |
| FTS F4 | % HMMS | 0.5 | 0.5 | 0.5 | 0.5 | 0.5 | 0.5 | 0.6 | 0.5 |
| | % Monomer | 99.5 | 99.5 | 99.5 | 99.6 | 99.5 | 99.5 | 99.4 | 99.5 |

TABLE 22
R-CGE

| Sample | | | Slow FT cycle | | | Fast FT cycle | | | |
|---|---|---|---|---|---|---|---|---|---|
| code | Attribute | Initial | 1 F/T | 3 F/T | 5 F/T | 1 F/T | 3 F/T | 5 F/T | Control |
| FTS F1 | % LC + HC | 86.3 | 95.8 | 93.6 | 97.1 | 94.8 | 97.9 | 97.0 | 96.9 |
| | % Fragments | 1.2 | 0.7 | 0.6 | 0.8 | 0.8 | 0.7 | 1.1 | 0.5 |
| | % Non-Reduced | 12.5 | 3.6 | 5.8 | 2.1 | 4.5 | 1.4 | 1.9 | 2.5 |
| FTS F2 | % LC + HC | 97.3 | 95.9 | 97.4 | 97.4 | 98.1 | 95.4 | 96.5 | 96.7 |
| | % Fragments | 0.1 | 0.3 | 0.6 | 0.6 | 0.3 | 0.2 | 0.5 | 0.4 |
| | % Non-Reduced | 2.5 | 3.9 | 2.0 | 2.0 | 1.6 | 4.4 | 3.0 | 2.9 |
| FTS F3 | % LC + HC | 95.6 | 95.1 | 93.0 | 94.4 | 94.9 | 94.2 | 94.0 | 95.5 |
| | % Fragments | 0.3 | 0.3 | 0.3 | 0.3 | 0.2 | 0.3 | 0.3 | 0.2 |
| | % Non-Reduced | 4.1 | 4.6 | 6.7 | 5.3 | 4.9 | 5.6 | 5.7 | 4.3 |
| FTS F4 | % LC + HC | 95.4 | 93.4 | 95.1 | 93.6 | 94.6 | 95.4 | 94.4 | 94.5 |
| | % Fragments | 0.2 | 0.2 | 0.1 | 0.2 | 0.2 | 0.3 | 0.3 | 0.3 |
| | % Non-Reduced | 4.4 | 6.4 | 4.8 | 6.2 | 5.2 | 4.3 | 5.3 | 5.3 |

TABLE 23
NR-CGE

| Sample | | | Slow FT cycle | | | Fast FT cycle | | | |
|---|---|---|---|---|---|---|---|---|---|
| code | Attribute | Initial | 1 F/T | 3 F/T | 5 F/T | 1 F/T | 3 F/T | 5 F/T | Control |
| FTS F1 | % Main peak | 91.0 | 92.0 | 84.2 | 86.1 | 89.3 | 94.2 | 91.5 | 87.9 |
| | % LMMS | 9.0 | 8.0 | 15.8 | 13.9 | 10.7 | 5.8 | 8.5 | 12.1 |
| FTS F2 | % Main peak | 94.9 | 94.4 | 93.7 | 95.0 | 94.3 | 93.3 | 95.0 | 93.6 |
| | % LMMS | 5.1 | 5.6 | 6.3 | 5.0 | 5.7 | 6.7 | 5.0 | 6.4 |
| FTS F3 | % Main peak | 92.7 | 92.8 | 93.3 | 93.8 | 94.7 | 91.7 | 93.3 | 95.1 |
| | % LMMS | 7.3 | 7.2 | 6.7 | 1.4 | 5.3 | 8.3 | 6.7 | 4.9 |
| FTS F4 | % Main peak | 94.7 | 93.9 | 94.5 | 94.8 | 94.4 | 93.6 | 94.2 | 94.9 |
| | % LMMS | 5.3 | 6.1 | 5.5 | 5.2 | 5.6 | 6.4 | 5.8 | 5.1 |

TABLE 24
IEX

| Sample | | | Slow FT cycle | | | Fast FT cycle | | | |
|---|---|---|---|---|---|---|---|---|---|
| code | Attributes | Initial | 1 F/T | 3 F/T | 5 F/T | 1 F/T | 3 F/T | 5 F/T | Control |
| FTS F1 | % Acidic | 7.5 | 7.1 | 7.4 | 7.6 | 7.5 | 6.5 | 7.3 | 7.2 |
| | % Main | 10.6 | 11.3 | 10.6 | 11.2 | 10.9 | 11.5 | 10.5 | 11.2 |
| | % Basic | 81.9 | 81.6 | 82.0 | 81.2 | 81.7 | 82.0 | 82.3 | 81.6 |
| FTS F2 | % Acidic | 10.4 | 10.2 | 10.6 | 11.4 | 9.4 | 10.8 | 10.8 | 10.0 |
| | % Main | 14.1 | 13.2 | 13.7 | 13.2 | 13.3 | 13.5 | 13.3 | 13.0 |
| | % Basic | 75.4 | 76.6 | 75.7 | 75.4 | 77.3 | 75.7 | 76.0 | 77.1 |
| FTS F3 | % Acidic | 8.0 | 7.3 | 7.3 | 7.7 | 7.2 | 7.3 | 7.3 | 7.8 |
| | % Main | 10.6 | 10.6 | 10.6 | 10.4 | 10.5 | 10.4 | 10.7 | 10.6 |
| | % Basic | 81.4 | 82.1 | 82.2 | 81.9 | 82.3 | 82.3 | 81.9 | 81.6 |
| FTS F4 | % Acidic | 8.1 | 7.5 | 7.6 | 7.8 | 7.4 | 7.3 | 6.9 | 7.7 |
| | % Main | 11.4 | 10.6 | 10.6 | 10.5 | 10.7 | 11.1 | 10.4 | 10.5 |
| | % Basic | 80.6 | 81.9 | 81.8 | 81.7 | 81.9 | 81.6 | 82.7 | 81.8 |

From the freeze/thaw studies, it was observed that the pH and concentration are consistent throughout freezing and thawing. Furthermore, all samples are clear, visible, and clear of particles throughout the study. The saline group has controlled acidic variants in this study. The Histidine/Histidine HCl buffer showed a good stable SEC profile with high percent monomer purity. And, at 0.1 mg/mL concentration the L-His/L-Histidine HCl buffer showed a stable NR-CGE profile with less percent LMMS compared to the saline formulation. However, at higher DS concentrations, both of the formulations have stabile profiles. As such, both formulations of saline and L-His/L-Histidine HCl offer significant stability enhancement for antibody storage. Nonetheless, L-His/L-Histidine HCl was found to be superior to saline in stabilizing antibodies during storage.

### Example 4: Alternative Formulations

A series of other formulations were screened for usage in antibody preparation and storage. The formulation were as depicted in Table 25.

TABLE 25
Compositions

| Component | Name | Composition | Final pH |
|---|---|---|---|
| Formulation | Saline 0.9% W/V | 9.00 g/L of Sodium Chloride | 5.8 ± 0.4 |
| 1 | | | |
| Formulation | 20 mM Histidine/Histidine | 3.10 g/L of L-Histidine. | 6.0 |
| 2 | HCl* | 4.18 g/L of L-Histidine HCl | |
| Formulation | 10 mM Citrate Phosphate^{#} | 1.78 g/L of disodium hydrogen phosphate | 6.0 |
| 3 | | dihydrate. 1.92 g/L of citric acid | |
| | | monohydrate | |
| Formulation | 10 mM Acetate^{#} | 0.96 g/L of Sodium acetate. | 5.0 |
| 4 | | 0.27 g/L of Acetic acid | |
| Excipient 1^{$} | Sucrose (85 mM) | 29.09 g/L of Sucrose (85 mM), | NA |
| | Sucrose (150 mM), | 51.35 g/L of sucrose (150 mM), | |
| | Sucrose (200 mM) | 68.46 g/L of sucrose (200 mM) | |
| Excipient 2^{$} | Poloxamer P188 (0.01% | 0.10 g/L of Poloxamer P188 | NA |
| | W/V) | | |

^{#}indicates the pH adjusted to respective pH.
^{$}indicates the excipients that have been used in combination with above formulations for higher stability.

What is claimed is:

1. An antibody formulation comprising histidine and an antibody, wherein the antibody targets P. gingivalis.

2. The antibody formulation of claim 1, wherein the antibody comprises at least one, two, three, four, five, or all six of the CDR regions of SEQ ID NOS: 1-2.

3. The antibody formulation of any one of claim 1 or 2, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

4. An antibody formulation comprising histidine and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

5. The antibody formulation of claim 4, wherein the antibody targets P. gingivalis.

6. The antibody formulation of any one of claim 4 or 5, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

7. The antibody formulation of any one of claims 4-6, wherein the antibody comprises a sequence that has at least 75%, 79%, or 80% identity to SEQ ID NO: 1 and a sequence that has at least 75%, 79%, or 80% identity to SEQ ID NO: 2.

8. The antibody formulation of any one of claims 1-7, wherein the formulation further comprises a sugar and/or a detergent.

9. The antibody formulation of claim 8, wherein the sugar is sucrose.

10. The antibody formulation of any one of claim 8 or 9, wherein the detergent is Poloxamer P188.

11. The antibody formulation of any one of claims 1-10, wherein the antibody is present at a concentration of about 0.9-0.12 mg/mL.

12. The antibody formulation of any one of the preceding claims, wherein the antibody is present at a concentration of 0.1 mg/mL.

13. The antibody formulation of any one of the preceding claims, wherein the histidine is present at a concentration of about 1 to about 40 mM.

14. The antibody formulation of any one of the preceding claims, wherein the histidine is present at a concentration of 20 mM.

15. The antibody formulation of any one of the preceding claims, wherein the formulation has a pH of about 4 to about 8.

16. The antibody formulation of any one of the preceding claims, wherein the formulation has a pH of 6.

17. The antibody formulation of any one of the preceding claims, wherein the formulation has a pH of 5.8.

18. The antibody formulation of any one of the preceding claims, wherein the formulation further comprises Poloxamer P188 at a concentration of about 0.001-0.1% weight/volume.

19. The antibody formulation of any one of the preceding claims, wherein the formulation further comprises Poloxamer P188 at a concentration of about 0.01% weight/volume.

20. The antibody formulation of any one of the preceding claims, wherein the formulation further comprises sucrose at a concentration of about 85 to 200 mM.

21. The antibody formulation of any one of the preceding claims, wherein the formulation further comprises sucrose at a concentration of about 85 mM.

22. The antibody formulation of any one of claims 1-20, wherein the formulation further comprises sucrose at a concentration of about 150 mM.

23. The antibody formulation of any one of claims 1-20, wherein the formulation further comprises sucrose at a concentration of about 200 mM.

24. An antibody formulation comprising histidine, sucrose, Poloxamer P188, and an antibody, wherein the antibody comprises at least one, two, three, four, five, six, seven, or all eight of:
a VH segment that is at least 75%, 79%, or 80% identical to SEQ ID NO:1;
a VL segment that is at least 75%, 79%, or 80% identical to SEQ ID NO: 2;
a CDR1 that is at least 75%, 79%, or 80% identical to the CDR1 of SEQ ID NO:1;
a CDR2 that is at least 75%, 79%, or 80% identical to the CDR2 of SEQ ID NO: 1;
a CDR3 that is at least 75%, 79%, or 80% identical to the CDR3 of SEQ ID NO: 1;
a CDR4 that is at least 75%, 79%, or 80% identical to the CDR4 of SEQ ID NO: 2;
a CDR5 that is at least 75%, 79%, or 80% identical to the CDR5 of SEQ ID NO: 2; and/or
a CDR6 that is at least 75%, 79%, or 80% identical to the CDR6 of SEQ ID NO: 2.

25. The antibody formulation of claim 24, wherein the antibody targets P. gingivalis.

26. The antibody formulation of any one of claim 24 or 25, wherein the antibody comprises a VH that has at least 75%, 79%, or 80% identity to a VH within SEQ ID NO: 1 and a VL that has at least 75%, 79%, or 80% identity to a VL within of SEQ ID NO: 2.

27. The antibody formulation of any one of claims 24-26, wherein the antibody comprises a sequence that has at least 75%, 79%, or 80% identity to SEQ ID NO: 1 and a sequence that has at least 75%, 79%, or 80% identity to SEQ ID NO: 2.

28. The antibody formulation of any one of claims 24-27, wherein histidine is present at a concentration of about 1 to about 40 mM.

29. The antibody formulation of any one of claims 24-28, wherein histidine is present at a concentration of about 20 mM.

30. The antibody formulation of any one of claims 24-29, wherein the formulation has a pH of about 4 to about 8.

31. The antibody formulation of any one of claims 24-30, wherein the formulation has a pH of 6.

32. The antibody formulation of any one of claims 24-31, wherein the formulation has a pH of 5.8.

33. The antibody formulation of any one of claims 24-32, wherein the Poloxamer P188 is present at a concentration of about 0.001-0.1% weight/volume.

34. The antibody formulation of any one of claims 24-33, wherein the Poloxamer P188 is present at a concentration of about 0.01% weight/volume.

35. The antibody formulation of any one of claims 24-34, wherein the sucrose is present at a concentration of about 85 to 200 mM.

36. The antibody formulation of any one of claims 24-35, wherein the sucrose is present at a concentration of about 85 mM.

37. The antibody formulation of any one of claims 24-35, wherein the sucrose is present at a concentration of about 150 mM.

38. The antibody formulation of any one of claims 24-35, wherein the sucrose is present at a concentration of about 200 mM.

39. An antibody formulation comprising an antibody, wherein the antibody comprises:
a CDR1 that is the CDR1 of SEQ ID NO:1;
a CDR2 that is the CDR2 of SEQ ID NO: 1;
a CDR3 that is the CDR3 of SEQ ID NO: 1;
a CDR4 that is the CDR4 of SEQ ID NO: 2;
a CDR5 that is the CDR5 of SEQ ID NO: 2; and
a CDR6 that is the CDR6 of SEQ ID NO: 2;
wherein the formulation is adjusted to a final pH of about 6.0;
wherein the formulation consists essentially of: 0.1 mg/mL antibody, 20 mM histidine, 0.01% Poloxamer P188, and sucrose at a concentration of about 85 mM to about 200 mM.

40. An antibody formulation comprising an antibody, wherein the antibody comprises:
a VH segment that has at least 75%, 79%, or 80% identity to SEQ ID NO:1;
and
a VL segment that has at least 75%, 79%, or 80% identity to SEQ ID NO: 2;
wherein the formulation is adjusted to a final pH of about 6.0;
wherein the formulation consists essentially of: 0.1 mg/mL antibody, 20 mM histidine, 0.01% Poloxamer P188, and sucrose at a concentration of about 85 mM to about 200 mM.

41. An antibody formulation, wherein the formulation consists essentially of: an antibody, a histidine, a detergent, and a sugar;
wherein the formulation is adjusted to a final pH of about 6.0;
wherein the antibody comprises:
a CDR1 that is the CDR1 of SEQ ID NO:1;
a CDR2 that is the CDR2 of SEQ ID NO: 1;
a CDR3 that is the CDR3 of SEQ ID NO: 1;
a CDR4 that is the CDR4 of SEQ ID NO: 2;
a CDR5 that is the CDR5 of SEQ ID NO: 2; and
a CDR6 that is the CDR6 of SEQ ID NO: 2.

42. An antibody formulation, wherein the formulation consists essentially of: an antibody, a histidine, a detergent, and a sugar;
wherein the formulation is adjusted to a final pH of about 6.0;
wherein the antibody comprises:
a VH segment that has at least 75%, 79%, or 80% identity to SEQ ID NO:1;
and
a VL segment that has at least 75%, 79%, or 80% identity to SEQ ID NO: 2.

43. The antibody formulation of any one of claim 41 or 42, wherein the detergent is Poloxamer P188.

44. The antibody formulation of any one of claims 41-43, wherein the sugar is sucrose.

45. The antibody formulation of any one of claims 41-44, wherein the formulation consists essentially of: 0.1 mg/mL antibody, 20 mM histidine, 0.01% Poloxamer P188, and sucrose at a concentration of about 85 mM to about 200 mM.

46. The antibody formulation of any one of the proceeding claims, wherein the antibody is KB001.

47. A method of storing an antibody, wherein the method comprises mixing the antibody with an antibody formulations comprising any one of the formulations of the previous claims.

48. A use of the any one of the antibody formulations of the previous claims as a storage formulation to enhance the stability of an at least one antibody or antigen binding molecule.

49. The method or use of any one of claim 47 or 48, wherein the antibody formulation is stored at below room temperature; preferably stored between about 20 to about −80° C.; most preferably stored at about 5° C.

50. The method or use of any one of claims 47-49, wherein the antibody formulation is stored as a solid and/or frozen.

51. The method or use of any one of claims 47-50, wherein the antibody formulation is stored as a liquid.

52. The antibody formulation or method or use of any one of the preceding claims, wherein the antibody comprises the heavy chain of SEQ ID NO: 1 and the light chain of SEQ ID NO: 2.


SEQUENCE LISTING (6 sequences)

SEQ ID NO: 1 - protein, 441 aa, Mus musculus
EVQLKQSGPGLVAPSWSLSITCTVSGFSLSIYSVHWVRQPPGKGLEWLGMIWGGGSSDYNSALKSRLSISKDNSKSQVFLKMNSLQTDDTAMYYCARNGNFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNCQPAENYKNTQPIMNTNGSYFVYSKLNVQKSNWEACNTFTCSFLHEGLHNHHTEKSLSHSPGK

SEQ ID NO: 2 - protein, 217 aa, Mus musculus
QIVLTQSPAIMSASLGERVTMTCTASSSVSSSFLHWYQQKPGSSPQLWIYSTSNLASGVPARFSGSGSGTSYSLTISSMEAEDAATYYCHQYHHSPYIYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 13 aa, Homo sapiens
YTYTVYRDGTKIK

SEQ ID NO: 4 - protein, 25 aa, Homo sapiens
GVSPKVCKDVTVEGSNEFAPVQNLT

SEQ ID NO: 5 - protein, 14 aa, Homo sapiens
YCVEVKYTAGVSPK

SEQ ID NO: 6 - protein, 22 aa, Homo sapiens
MDFQVQIFSFLLISASVIMSRG